SEQUENCE LISTING (118 sequences)

SEQ ID NO: 1 - protein, 40 aa, Homo sapiens
  MOD_RES (40)..(40): AMIDATION
TKFTLSLDVPTNIMNLLFNIAKAKNLRAQAAANAHLMAQI

SEQ ID NO: 2 - protein, 40 aa, Artificial Sequence
  Synthetic peptide
CKFTLSLDVPTNIMNLLFNIAKAKNLRAQAAANAHLMAQI

SEQ ID NO: 3 - protein, 40 aa, Artificial Sequence
  Synthetic peptide
TCFTLSLDVPTNIMNLLFNIAKAKNLRAQAAANAHLMAQI

SEQ ID NO: 4 - protein, 40 aa, Artificial Sequence
  Synthetic peptide
TKCTLSLDVPTNIMNLLFNIAKAKNLRAQAAANAHLMAQI

SEQ ID NO: 5 - protein, 40 aa, Artificial Sequence
  Synthetic peptide
TKFCLSLDVPTNIMNLLFNIAKAKNLRAQAAANAHLMAQI

SEQ ID NO: 6 - protein, 40 aa, Artificial Sequence
  Synthetic peptide
TKFTCSLDVPTNIMNLLFNIAKAKNLRAQAAANAHLMAQI

SEQ ID NO: 7 - protein, 40 aa, Artificial Sequence
  Synthetic peptide
TKFTLCLDVPTNIMNLLFNIAKAKNLRAQAAANAHLMAQI

SEQ ID NO: 8 - protein, 40 aa, Artificial Sequence
  Synthetic peptide
TKFTLSCDVPTNIMNLLFNIAKAKNLRAQAAANAHLMAQI

SEQ ID NO: 9 - protein, 40 aa, Artificial Sequence
  Synthetic peptide
TKFTLSLCVPTNIMNLLFNIAKAKNLRAQAAANAHLMAQI

SEQ ID NO: 10 - protein, 40 aa, Artificial Sequence
  Synthetic peptide
TKFTLSLDCPTNIMNLLFNIAKAKNLRAQAAANAHLMAQI

SEQ ID NO: 11 - protein, 40 aa, Artificial Sequence
  Synthetic peptide
TKFTLSLDVCTNIMNLLFNIAKAKNLRAQAAANAHLMAQI

SEQ ID NO: 12 - protein, 40 aa, Artificial Sequence
  Synthetic peptide
TKFTLSLDVPCNIMNLLFNIAKAKNLRAQAAANAHLMAQI

SEQ ID NO: 13 - protein, 40 aa, Artificial Sequence
  Synthetic peptide
TKFTLSLDVPTCIMNLLFNIAKAKNLRAQAAANAHLMAQI

SEQ ID NO: 14 - protein, 40 aa, Artificial Sequence
  Synthetic peptide
TKFTLSLDVPTNCMNLLFNIAKAKNLRAQAAANAHLMAQI

SEQ ID NO: 15 - protein, 40 aa, Artificial Sequence
  Synthetic peptide
TKFTLSLDVPTNICNLLFNIAKAKNLRAQAAANAHLMAQI

SEQ ID NO: 16 - protein, 40 aa, Artificial Sequence
  Synthetic peptide
TKFTLSLDVPTNIMCLLFNIAKAKNLRAQAAANAHLMAQI

SEQ ID NO: 17 - protein, 40 aa, Artificial Sequence
  Synthetic peptide
TKFTLSLDVPTNIMNCLFNIAKAKNLRAQAAANAHLMAQI

SEQ ID NO: 18 - protein, 40 aa, Artificial Sequence
  Synthetic peptide
TKFTLSLDVPTNIMNLCFNIAKAKNLRAQAAANAHLMAQI

SEQ ID NO: 19 - protein, 40 aa, Artificial Sequence
  Synthetic peptide
TKFTLSLDVPTNIMNLLCNIAKAKNLRAQAAANAHLMAQI

SEQ ID NO: 20 - protein, 40 aa, Artificial Sequence
  Synthetic peptide
TKFTLSLDVPTNIMNLLFCIAKAKNLRAQAAANAHLMAQI

SEQ ID NO: 21 - protein, 40 aa, Artificial Sequence
  Synthetic peptide
TKFTLSLDVPTNIMNLLFNCAKAKNLRAQAAANAHLMAQI

SEQ ID NO: 22 - protein, 40 aa, Artificial Sequence
  Synthetic peptide
TKFTLSLDVPTNIMNLLFNICKAKNLRAQAAANAHLMAQI

SEQ ID NO: 23 - protein, 40 aa, Artificial Sequence
  Synthetic peptide
TKFTLSLDVPTNIMNLLFNIACAKNLRAQAAANAHLMAQI

SEQ ID NO: 24 - protein, 40 aa, Artificial Sequence
  Synthetic peptide
TKFTLSLDVPTNIMNLLFNIAKCKNLRAQAAANAHLMAQI

SEQ ID NO: 25 - protein, 40 aa, Artificial Sequence
  Synthetic peptide
TKFTLSLDVPTNIMNLLFNIAKACNLRAQAAANAHLMAQI

SEQ ID NO: 26 - protein, 40 aa, Artificial Sequence
  Synthetic peptide
TKFTLSLDVPTNIMNLLFNIAKAKCLRAQAAANAHLMAQI

SEQ ID NO: 27 - protein, 40 aa, Artificial Sequence
  Synthetic peptide
TKFTLSLDVPTNIMNLLFNIAKAKNCRAQAAANAHLMAQI

SEQ ID NO: 28 - protein, 40 aa, Artificial Sequence
  Synthetic peptide
TKFTLSLDVPTNIMNLLFNIAKAKNLCAQAAANAHLMAQI

SEQ ID NO: 29 - protein, 40 aa, Artificial Sequence
  Synthetic peptide
TKFTLSLDVPTNIMNLLFNIAKAKNLRCQAAANAHLMAQI

SEQ ID NO: 30 - protein, 40 aa, Artificial Sequence
  Synthetic peptide
TKFTLSLDVPTNIMNLLFNIAKAKNLRACAAANAHLMAQI

SEQ ID NO: 31 - protein, 40 aa, Artificial Sequence
  Synthetic peptide
TKFTLSLDVPTNIMNLLFNIAKAKNLRAQCAANAHLMAQI

SEQ ID NO: 32 - protein, 40 aa, Artificial Sequence
  Synthetic peptide
TKFTLSLDVPTNIMNLLFNIAKAKNLRAQACANAHLMAQI

SEQ ID NO: 33 - protein, 40 aa, Artificial Sequence
  Synthetic peptide
TKFTLSLDVPTNIMNLLFNIAKAKNLRAQAACNAHLMAQI

SEQ ID NO: 34 - protein, 40 aa, Artificial Sequence
  Synthetic peptide
TKFTLSLDVPTNIMNLLFNIAKAKNLRAQAAACAHLMAQI

SEQ ID NO: 35 - protein, 40 aa, Artificial Sequence
  Synthetic peptide
TKFTLSLDVPTNIMNLLFNIAKAKNLRAQAAANCHLMAQI

SEQ ID NO: 36 - protein, 40 aa, Artificial Sequence
  Synthetic peptide
TKFTLSLDVPTNIMNLLFNIAKAKNLRAQAAANACLMAQI

SEQ ID NO: 37 - protein, 40 aa, Artificial Sequence
  Synthetic peptide
TKFTLSLDVPTNIMNLLFNIAKAKNLRAQAAANAHCMAQI

SEQ ID NO: 38 - protein, 40 aa, Artificial Sequence
  Synthetic peptide
TKFTLSLDVPTNIMNLLFNIAKAKNLRAQAAANAHLCAQI

SEQ ID NO: 39 - protein, 40 aa, Artificial Sequence
  Synthetic peptide
TKFTLSLDVPTNIMNLLFNIAKAKNLRAQAAANAHLMCQI

SEQ ID NO: 40 - protein, 40 aa, Artificial Sequence
  Synthetic peptide
TKFTLSLDVPTNIMNLLFNIAKAKNLRAQAAANAHLMACI

SEQ ID NO: 41 - protein, 40 aa, Artificial Sequence
  Synthetic peptide
TKFTLSLDVPTNIMNLLFNIAKAKNLRAQAAANAHLMAQC

SEQ ID NO: 42 - protein, 40 aa, Artificial Sequence; identical to SEQ ID NO: 41
  Synthetic peptide

SEQ ID NO: 43 - protein, 40 aa, Artificial Sequence; identical to SEQ ID NO: 36
  Synthetic peptide

SEQ ID NO: 44 - protein, 40 aa, Artificial Sequence; identical to SEQ ID NO: 34
  Synthetic peptide

SEQ ID NO: 45 - protein, 40 aa, Artificial Sequence; identical to SEQ ID NO: 33
  Synthetic peptide

SEQ ID NO: 46 - protein, 40 aa, Artificial Sequence; identical to SEQ ID NO: 32
  Synthetic peptide

SEQ ID NO: 47 - protein, 40 aa, Artificial Sequence; identical to SEQ ID NO: 29
  Synthetic peptide

SEQ ID NO: 48 - protein, 40 aa, Artificial Sequence; identical to SEQ ID NO: 27
  Synthetic peptide

SEQ ID NO: 49 - protein, 40 aa, Artificial Sequence; identical to SEQ ID NO: 26
  Synthetic peptide

SEQ ID NO: 50 - protein, 40 aa, Artificial Sequence; identical to SEQ ID NO: 25
  Synthetic peptide

SEQ ID NO: 51 - protein, 40 aa, Artificial Sequence; identical to SEQ ID NO: 21
  Synthetic peptide

SEQ ID NO: 52 - protein, 40 aa, Artificial Sequence; identical to SEQ ID NO: 20
  Synthetic peptide

SEQ ID NO: 53 - protein, 40 aa, Artificial Sequence; identical to SEQ ID NO: 18
  Synthetic peptide

SEQ ID NO: 54 - protein, 40 aa, Artificial Sequence; identical to SEQ ID NO: 17
  Synthetic peptide

SEQ ID NO: 55 - protein, 40 aa, Artificial Sequence; identical to SEQ ID NO: 2
  Synthetic peptide

SEQ ID NO: 56 - protein, 40 aa, Artificial Sequence; identical to SEQ ID NO: 3
  Synthetic peptide

SEQ ID NO: 57 - protein, 40 aa, Artificial Sequence; identical to SEQ ID NO: 4
  Synthetic peptide

SEQ ID NO: 58 - protein, 40 aa, Artificial Sequence; identical to SEQ ID NO: 5
  Synthetic peptide

SEQ ID NO: 59 - protein, 40 aa, Artificial Sequence; identical to SEQ ID NO: 6
  Synthetic peptide

SEQ ID NO: 60 - protein, 40 aa, Artificial Sequence; identical to SEQ ID NO: 7
  Synthetic peptide

SEQ ID NO: 61 - protein, 40 aa, Artificial Sequence; identical to SEQ ID NO: 8
  Synthetic peptide

SEQ ID NO: 62 - protein, 40 aa, Artificial Sequence; identical to SEQ ID NO: 9
  Synthetic peptide

SEQ ID NO: 63 - protein, 40 aa, Artificial Sequence; identical to SEQ ID NO: 10
  Synthetic peptide

SEQ ID NO: 64 - protein, 40 aa, Artificial Sequence; identical to SEQ ID NO: 11
  Synthetic peptide

SEQ ID NO: 65 - protein, 40 aa, Artificial Sequence; identical to SEQ ID NO: 12
  Synthetic peptide

SEQ ID NO: 66 - protein, 40 aa, Artificial Sequence; identical to SEQ ID NO: 13
  Synthetic peptide

SEQ ID NO: 67 - protein, 40 aa, Artificial Sequence; identical to SEQ ID NO: 14
  Synthetic peptide

SEQ ID NO: 68 - protein, 40 aa, Artificial Sequence; identical to SEQ ID NO: 15
  Synthetic peptide

SEQ ID NO: 69 - protein, 40 aa, Artificial Sequence; identical to SEQ ID NO: 16
  Synthetic peptide

SEQ ID NO: 70 - protein, 40 aa, Artificial Sequence; identical to SEQ ID NO: 17
  Synthetic peptide

SEQ ID NO: 71 - protein, 40 aa, Artificial Sequence; identical to SEQ ID NO: 18
  Synthetic peptide

SEQ ID NO: 72 - protein, 40 aa, Artificial Sequence; identical to SEQ ID NO: 19
  Synthetic peptide

SEQ ID NO: 73 - protein, 40 aa, Artificial Sequence; identical to SEQ ID NO: 20
  Synthetic peptide

SEQ ID NO: 74 - protein, 40 aa, Artificial Sequence; identical to SEQ ID NO: 21
  Synthetic peptide

SEQ ID NO: 75 - protein, 40 aa, Artificial Sequence; identical to SEQ ID NO: 22
  Synthetic peptide

SEQ ID NO: 76 - protein, 40 aa, Artificial Sequence; identical to SEQ ID NO: 23
  Synthetic peptide

SEQ ID NO: 77 - protein, 40 aa, Artificial Sequence; identical to SEQ ID NO: 24
  Synthetic peptide

SEQ ID NO: 78 - protein, 40 aa, Artificial Sequence; identical to SEQ ID NO: 25
  Synthetic peptide

SEQ ID NO: 79 - protein, 40 aa, Artificial Sequence; identical to SEQ ID NO: 26
  Synthetic peptide

SEQ ID NO: 80 - protein, 40 aa, Artificial Sequence; identical to SEQ ID NO: 27
  Synthetic peptide

SEQ ID NO: 81 - protein, 40 aa, Artificial Sequence; identical to SEQ ID NO: 28
  Synthetic peptide

SEQ ID NO: 82 - protein, 40 aa, Artificial Sequence; identical to SEQ ID NO: 29
  Synthetic peptide

SEQ ID NO: 83 - protein, 40 aa, Artificial Sequence; identical to SEQ ID NO: 30
  Synthetic peptide

SEQ ID NO: 84 - protein, 40 aa, Artificial Sequence; identical to SEQ ID NO: 31
  Synthetic peptide

SEQ ID NO: 85 - protein, 40 aa, Artificial Sequence; identical to SEQ ID NO: 32
  Synthetic peptide

SEQ ID NO: 86 - protein, 40 aa, Artificial Sequence; identical to SEQ ID NO: 33
  Synthetic peptide

SEQ ID NO: 87 - protein, 40 aa, Artificial Sequence; identical to SEQ ID NO: 34
  Synthetic peptide

SEQ ID NO: 88 - protein, 40 aa, Artificial Sequence; identical to SEQ ID NO: 35
  Synthetic peptide

SEQ ID NO: 89 - protein, 40 aa, Artificial Sequence; identical to SEQ ID NO: 36
  Synthetic peptide

SEQ ID NO: 90 - protein, 40 aa, Artificial Sequence; identical to SEQ ID NO: 37
  Synthetic peptide

SEQ ID NO: 91 - protein, 40 aa, Artificial Sequence; identical to SEQ ID NO: 38
  Synthetic peptide

SEQ ID NO: 92 - protein, 40 aa, Artificial Sequence; identical to SEQ ID NO: 39
  Synthetic peptide

SEQ ID NO: 93 - protein, 40 aa, Artificial Sequence; identical to SEQ ID NO: 40
  Synthetic peptide

SEQ ID NO: 94 - protein, 40 aa, Artificial Sequence; identical to SEQ ID NO: 41
  Synthetic peptide

SEQ ID NO: 95 - protein, 40 aa, Artificial Sequence; identical to SEQ ID NO: 29
  Synthetic peptide

SEQ ID NO: 96 - protein, 40 aa, Artificial Sequence; identical to SEQ ID NO: 29
  Synthetic peptide

SEQ ID NO: 97 - protein, 40 aa, Artificial Sequence; identical to SEQ ID NO: 29
  Synthetic peptide

SEQ ID NO: 98 - protein, 40 aa, Artificial Sequence; identical to SEQ ID NO: 29
  Synthetic peptide

SEQ ID NO: 99 - protein, 40 aa, Artificial Sequence; identical to SEQ ID NO: 29
  Synthetic peptide

SEQ ID NO: 100 - protein, 40 aa, Artificial Sequence; identical to SEQ ID NO: 29
  Synthetic peptide

SEQ ID NO: 101 - protein, 40 aa, Artificial Sequence; identical to SEQ ID NO: 29
  Synthetic peptide

SEQ ID NO: 102 - protein, 40 aa, Artificial Sequence; identical to SEQ ID NO: 29
  Synthetic peptide

SEQ ID NO: 103 - protein, 40 aa, Artificial Sequence; identical to SEQ ID NO: 29
  Synthetic peptide

SEQ ID NO: 104 - protein, 40 aa, Artificial Sequence; identical to SEQ ID NO: 29
  Synthetic peptide

SEQ ID NO: 105 - protein, 40 aa, Artificial Sequence; identical to SEQ ID NO: 13
  Synthetic peptide

SEQ ID NO: 106 - protein, 40 aa, Artificial Sequence; identical to SEQ ID NO: 36
  Synthetic peptide

SEQ ID NO: 107 - protein, 39 aa, Artificial Sequence
  Synthetic peptide
KFTLSLDVPTNIMNLLFNIAKAKNLRAQAAANAHLMAQI

SEQ ID NO: 108 - protein, 37 aa, Artificial Sequence
  Synthetic peptide
TLSLDVPTNIMNLLFNIAKAKNLRAQAAANAHLMAQI

SEQ ID NO: 109 - protein, 36 aa, Artificial Sequence
  Synthetic peptide
LSLDVPTNIMNLLFNIAKAKNLRAQAAANAHLMAQI

SEQ ID NO: 110 - protein, 35 aa, Artificial Sequence
  Synthetic peptide
SLDVPTNIMNLLFNIAKAKNLRAQAAANAHLMAQI

SEQ ID NO: 111 - protein, 34 aa, Artificial Sequence
  Synthetic peptide
LDVPTNIMNLLFNIAKAKNLRAQAAANAHLMAQI

SEQ ID NO: 112 - protein, 33 aa, Artificial Sequence
  Synthetic peptide
DVPTNIMNLLFNIAKAKNLRAQAAANAHLMAQI

SEQ ID NO: 113 - protein, 40 aa, Homo sapiens; identical to SEQ ID NO: 1

SEQ ID NO: 114 - protein, 40 aa, Rattus norvegicus
  MOD_RES (40)..(40): AMIDATION
DDPPLSIDLTFHLLRTLLELARTQSQRERAEQNRIIFDSV

SEQ ID NO: 115 - protein, 38 aa, Homo sapiens
  MOD_RES (38)..(38): AMIDATION
IVLSLDVPIGLLQILLEQARARAAREQATTNARILARV

SEQ ID NO: 116 - protein, 38 aa, Homo sapiens
  MOD_RES (38)..(38): AMIDATION
FTLSLDVPTNIMNLLFNIAKAKNLRAQAAANAHLMAQI

SEQ ID NO: 117 - protein, 43 aa, Homo sapiens
  MOD_RES (43)..(43): AMIDATION
HPGSRIVLSLDVPIGLLQILLEQARARAAREQATTNARILARV

SEQ ID NO: 118 - protein, 30 aa, Artificial Sequence
  Synthetic peptide
FHLLRKMIEIEKQEKEKQQAANNRLLLDTI

(19) United States
(12) Patent Application Publication
(10) Pub. No.: US 2011/0105397 A1
(43) Pub. Date: May 5, 2011

(54) METHOD FOR TREATING HEART FAILURE WITH STRESSCOPIN-LIKE PEPTIDES

(76) Inventors: Peter J. Gengo, Carlsbad, CA (US); Hani N. Sabbah, Waterford, MI (US); Nigel P. Shankley, Solana Beach, CA (US)

(21) Appl. No.: 12/940,019

(22) Filed: Nov. 4, 2010

Related U.S. Application Data
(60) Provisional application No. 61/258,181, filed on Nov. 4, 2009.

(51) Int. Cl. A61K 38/22; A61P 9/04

(52) U.S. Cl. 514/9.7

(57) ABSTRACT

The present invention relates to novel methods of treating heart failure comprising administering an amount of stresscopin-like peptide to a subject in need thereof; and substantially maintaining the amount of said peptide present in the plasma of said subject at a concentration resulting in a therapeutic benefit without a substantial increase in the heart rate of said subject. The method involves the use of stresscopin-like peptides that are selective corticotrophin releasing hormone receptor type 2 (CRHR2) agonists.

### CROSS REFERENCE TO RELATED APPLICATIONS

This application claims the benefit of U.S. provisional patent application Ser. No. 61/258,181, filed Nov. 4, 2009 and U.S. national patent application Ser. No. 12/612,548, filed Nov. 4, 2009, which are hereby incorporated by reference.

### FIELD OF THE INVENTION

This invention relates to methods of treating a subject for heart failure by administering an effective amount of a stresscopin-like polypeptide.

### BACKGROUND

Heart failure is a common cardiovascular condition and has reached epidemic proportions in the United States and Europe (Remme et al., Eur. Heart J., 2001, vol. 22, pp. 1527-1560). The number of hospital admissions for acute heart failure is approaching 1 million each year in the United States alone. Currently, readmission rates and mortality have reached 30% to 40% within 60 days following discharge (Cuffee et al., JAMA, 2002, vol. 287(12), pp. 1541-7). In acute heart failure, worsening of hemodynamic function, in particular with very high left ventricular end-diastolic pressure is common.

The current treatment for acute heart failure is multifactorial and often differs among patients. While diuretics, vasodilators, and positive inotropes remain the mainstay in the treatment of patients with acute heart failure, these treatments are associated with mortality and high readmission rates.

Furthermore, existing inotropic therapies (eg, dobutamine) result in improved cardiac output, but with increased heart rate and increased myocardial oxygen consumption. These inotropic agents also carry with them a proarrhythmic potential in patients with heart failure. This cardiac liability is believed to be associated with the energy expense and calcium drive associated with these agents' direct positive inotropic actions.

In an effort to meet this growing unmet medical need, many new approaches have been studied with limited success in safely improving the hemodynamic status and outcome of patients with this syndrome. One such agent, the peptide human urocortin 2 (h-UCN2), has been studied in healthy subjects and patients with heart failure. This peptide was shown to increase left ventricular ejection fraction (LVEF) and cardiac output (CO) in a model of heart failure in sheep (Rademaker et al., Circulation, 2005, vol. 112, pp. 3624-3632). In subsequent intravenous infusion studies in 8 healthy subjects (Davis et al., J. Am. Coll. Cardiol., 2007, vol. 49, pp. 461-471) and in 8 subjects with heart failure (Davis et al., Eur. Heart J., 2007, vol. 28, pp. 2589-2597), the increases in LVEF and CO were accompanied by an increase in heart rate and decrease in blood pressure at both doses examined in each of the two studies. One-hour intravenous infusions of h-UCN2 in healthy subjects and patients appears to have been well tolerated.

Human stresscopin (h-SCP), a 40-amino-acid peptide, is related to h-UCN2 and both are members of the corticotrophin releasing hormone (CRH) peptide family. The biological actions of the CRH peptide family are elicited by two 7 transmembrane G-protein coupled receptors, CRH receptor type 1 (CRHR1) and CRH receptor type 2 (CRHR2). Although these receptors contain high sequence homology, the different members of the CRH peptide family express significant differences in their relative binding affinity, degree of receptor activation and selectivity for these two receptors.

Human urocortin 2 (h-UCN2), was evaluated in previous intravenous infusion studies (Davis et al., J. Am. Coll. Cardiol., 2007, vol. 49, pp. 461-471; Davis et al., Eur. Heart J., 2007, vol. 28, pp. 2589-2597) of healthy and heart failure subjects and caused increases in LVEF and CO in the subjects that were accompanied by a significant increase in heart rate and decrease in blood pressure. The dose rates for healthy subjects were 5.16 ng/kg/min and 20.8 ng/kg/min, whereas h-UCN2 was infused at a rate of 4.29 ng/kg/min and 17.2 ng/kg/min to heart failure subjects.

Unlike many of the CRH family members, h-SCP expresses greater selectivity for the CRHR2 and acts as a mediator that aids in the process of attenuating the initiation and maintenance of physiological stress (Bale et al., Nat. Genet., 2000, vol. 24, pp. 410-414; Kishimoto et al., Nat. Genet., 2000, vol. 24, pp. 415-419). In addition to its apparent role in physiological stress, h-SCP has been reported to elicit a number of other physiological actions. It exerts effects on the endocrine (Li et al., Endocrinology, 2003, vol. 144, pp. 3216-3224), central nervous, cardiovascular (Bale et al., Proc. Natl. Acad. Sci., 2004, vol. 101, pp. 3697-3702; Tang et al., Eur. Heart J., 2007, vol. 28, pp. 2561-2562), pulmonary, gastrointestinal, renal, skeletal muscle, and inflammatory systems (Moffatt et al., FASEB J., 2006, vol. 20, pp. 1877-1879).

In addition, CRHR2 activity has been implicated in skeletal muscle wasting disease, such as sarcopenia (Hinkle et al., Endocrinology, 2003, vol. 144(11), pp. 4939-4946), motor activity and food intake (Ohata et al., Peptides, 2004, vol. 25, pp. 1703-1709), participates in a cardioprotective role (Brar et al., Endocrinology, 2004, vol. 145(1), pp. 24-35) and expresses bronchorelaxant and anti-inflammatory activity (Moffatt et al., FASEB J., 2006, vol. 20, pp. E1181-E1187).

Pegylation is a process of attaching one or more polyethylene glycol (PEG) polymers to molecules. Often, the process of pegylation is applied to antibodies, peptides and proteins to improve their biopharmaceutical properties and overcome a compound's susceptibility to proteolytic enzymes, short circulation half-life, short shelf live, low solubility, rapid renal clearance and the potential to generate antibodies to the administered drug (Harris et al., Nature, 2003,vol. 2, pp. 214-221; Hamidi et al., Drug Delivery, 2006, 3, pp. 399-409; Bailon et al., PSTT, 1998, vol. 1(8), pp. 352-356). Recently, the FDA has approved PEG polymers for use as a vehicle or base in foods, cosmetics, and pharmaceuticals. Overall, PEG polymers are relatively non-immunogenic, have little toxicity, and are eliminated intact by the kidneys or in the feces. These features can result in a number of clinical benefits for the compound if this process is developed to preserve or improve the affinity, efficacy and pharmacologic profile of the parent molecule.

### SUMMARY OF THE INVENTION

The invention is directed to the general and preferred embodiments defined, respectively, by the independent and dependent claims appended hereto, which are incorporated by reference herein. Preferred and exemplary features of the invention will be apparent from the detailed description below with reference to the drawing figures.

In its many embodiments, the present invention relates to a novel method of treating a heart failure patient. A method of treatment, prevention, inhibition or amelioration of one or more diseases associated with CRHR2 and related to heart failure using stresscopin-like peptides is provided.

The method for treating heart failure comprises administering an amount of stresscopin-like peptide to a subject in need thereof, and substantially maintaining the amount of said peptide present in the plasma of said subject at concentrations that result in a therapeutic benefit without a substantial increase in the heart rate of said subject.

In one embodiment of the treatment method, the plasma level of the stresscopin-like peptide in said subject is substantially maintained at concentrations that result in an increase in cardiac performance without a significant increase in the heart rate or a significant decrease in blood pressure of said subject.

In one embodiment, upon administration the stresscopin-relative blood plasma concentration profile of the stresscopin-like peptide is characterized by the plasma concentration substantially maintained below about 7.2 ng/mL, preferably below about 5.5 ng/mL, more preferably below about 4.7 ng/mL. The stresscopin-relative concentration of a peptide is the concentration that is weight and CRHR2 activity equivalent to a concentration amount of the stresscopin-like peptide of the following sequence (SEQ ID NO:1):

TKFTL SLDVP TNIMN LLFNI AKAKN LRAQA AANAH LMAQI-NH₂.

Preferably, the stresscopin-like peptide is administered to achieve a target stresscopin-relative blood plasma concentration profile of the peptide that is characterized by the plasma concentration substantially maintained between about 0.1 ng/mL to about 7.2 ng/mL. More preferably, the administration of stresscopin-like peptide leads to a stresscopin-relative blood plasma concentration profile with a plasma concentration between about 0.1 ng/mL to about 5.5 ng/mL.

An advantage of administering stresscopin-like peptides to a subject yielding a stresscopin-relative blood plasma concentration profile with a plasma concentration substantially maintained below about 7.2 ng/mL is that the treatment results in an increase in cardiac performance without a significant increase in heart rate or significant decrease in blood pressure of the subject.

The administration for treating heart failure is preferably via a parenteral route including intravenous, subcutaneous or intramuscular administration. These administration routes are advantageous, since they allow for more incremental control over the administered dose of stresscopin-like peptide in order to substantially maintain a plasma concentration that is below about 7.2 ng/mL in the stresscopin-relative blood plasma concentration profile.

In particular embodiments of the present invention, a stresscopin-like peptide comprises a peptide of SEQ ID NO:1 (h-SCP). In other embodiments it comprises a modified h-SCP, wherein h-SCP has been modified by covalent attachment of a reactive group, by conservative amino acid substitution, deletion or addition, by pegylation, or a combination of all of these modifications.

In yet other embodiments, the stresscopin-like peptide comprises an optical isomer, enantiomer, diastereomer, tautomer, cis-trans isomer, racemate, prodrug or pharmaceutically acceptable salt of h-SCP or its modifications.

In another embodiment, the reactive group also comprises a linker. Preferably only one linker is attached to a single residue in the amino acid sequence of the peptide. More preferably, the linker is acetamide or N-ethylsuccinimide.

In yet another embodiment, the stresscopin-like peptide comprises one or more PEG moieties that possess a molecular weight of less than 80 kDa. Preferably, the PEG moiety is covalently attached to the peptide. More preferably, the one or more PEG moieties are attached to the peptide through a linker. Even more preferably, the PEG moiety has a molecular weight of either about 2 kDa, about 5 kDa, about 12 kDa, about 20 kDa, about 30 kDa or about 40 kDa.

A linker allows for more easily and selectively attaching the PEG moiety with regard to the position in the amino acid sequence to the peptide, while pegylation of the peptide prolongs the half-life of the pegylated peptide, thereby extending the duration of therapeutic benefit to a patient. Therefore, the modification to the amino acid sequence of the stresscopin-like peptide is preferably such that there is only one amino acid of type X in the sequence. This will ensure that pegylation of the peptide is directed only to a single position in the sequence.

The benefits of a pegylated stresscopin-like peptide include a prolonged half-life of the pegylated peptide that insures that the plasma concentration of the stresscopin-relative blood plasma concentration profile is substantially maintained below about 7.2 ng/mL and stays for a longer time in the target range for the stresscopin-relative blood plasma concentration than the unpegylated stresscopin-like peptide, thereby extending the duration of therapeutic benefit to the patient.

Another embodiment of the present invention features the administration of a pharmaceutical composition comprising at least one compound of the present invention.

Additional embodiments and advantages of the invention will become apparent from the detailed discussion, schemes, examples, and claims below.

### BRIEF DESCRIPTION OF THE FIGURES

FIG. 1 illustrates the blood plasma profile and therapeutic window for administering a stresscopin-like peptide in order to treat heart failure patients.

FIGS. 2A, B & C illustrate the therapeutic window and blood plasma profile utilizing different routes of administering stresscopin-like peptides.

FIGS. 3A & B show the analytical HPLC trace of a stresscopin-like peptide with SEQ ID NO:102 derivatized with iodoacetamide-PEG after 2 hours reaction time and after purification, respectively.

FIG. 3C shows the mass spectroscopy graph of a stresscopin-like peptide with SEQ ID NO:102 that was derivatized with iodoacetamide-PEG.

FIG. 4 shows the agonist potency and selectivity of stresscopin-like peptides against human CRHR1 and CRHR2, respectively.

FIG. 5 displays the effects of competitive antagonism between a stresscopin-like peptide with SEQ ID NO:1 and anti-sauvagine-30 (SEQ ID NO:118).

FIG. 6 shows agonist concentration-effect curves of various stresscopin-like peptides obtained by measuring cAMP stimulation in h-CRHR2 transfected SK-N-MC cells.

FIG. 7 displays the h-SCP (SEQ ID NO:1) agonist concentration-effect curves measured through cAMP stimulation in h-CRHR2 transfected SK-N-MC cells in the absence and presence of 10 μM of stresscopin-like peptides with sequence SEQ ID NO:110, SEQ ID NO:111 and SEQ ID NO:112, respectively.

FIG. 8 shows the relaxation of precontracted, isolated rat aorta by stresscopin-like peptides with SEQ ID NO:1 and SEQ ID NO:115 (h-UCN2).

FIG. 9 illustrates the heart rate, left ventricular developed pressure, and coronary perfusion pressure changes in Langendorff perfused rabbit hearts in the presence of stresscopin-like peptide with SEQ ID NO:1 and placebo control vehicle.

FIG. 10 illustrates the effects of the stresscopin-like peptide with SEQ ID NO:1 administered by IV bolus injection on heart rate, mean artery blood pressure (MAP), and left ventricular contractility (+dP/dt) in anaesthetized rats.

FIGS. 11A & B shows the cardiac performance of healthy dogs upon intravenous infusion at different dose rates of a stresscopin-like peptide with SEQ ID NO:1.

FIGS. 12A & B shows the cardiac performance of dogs with induced heart failure upon intravenous infusion at different dose rates of a stresscopin-like peptide with SEQ ID NO:1.

FIG. 12C shows the cardiac performance for HF dogs in case of a single SC bolus injection of a stresscopin-like peptide with SEQ ID NO:102.

FIGS. 13A & B illustrates the pharmacokinetics of a stresscopin-like peptide with SEQ ID NO:102 in dogs following intravenous or subcutaneous bolus injection of different doses.

FIG. 13C illustrates the pharmacokinetics of a stresscopin-like peptide with SEQ ID NO:1 in dogs following intravenous dosing over 3 hours at various dose rates.

FIGS. 14A and B shows representative LV pressure-volume loops in dogs with heart failure (A) in the absence and (B) following a 2-hour infusion of stresscopin-like peptide with SEQ ID NO:1.

FIG. 15A illustrates the pharmacokinetics of a stresscopin-like peptide with SEQ ID NO:1 in rats through intravenous or subcutaneous bolus injection.

FIG. 15B to E illustrate the pharmacokinetics of pegylated stresscopin-like peptides (SEQ ID NO:102, 103, 104, 105, and 106) in rats following intravenous or subcutaneous bolus injection of different doses.

FIG. 16A to C shows the mean plasma concentration of a stresscopin-like peptide with SEQ ID NO:1 following 7.5-hour intravenous infusions in (A) healthy subjects, (B) in subjects with heart failure, and (C) following an infusion of 54 ng/kg/min in healthy subjects.

FIG. 17 shows the heart rate of healthy placebo subjects over time during a 7.5-hour intravenous infusion study of a stresscopin-like peptide with SEQ ID NO:1.

FIG. 18A to C shows change in (A) heart rate, (B) in cardiac index, and (C) in stroke volume, for healthy versus heart-failure subjects during a 7.5-hour intravenous infusion of of a stresscopin-like peptide with SEQ ID NO:1.

FIG. 19 shows change in heart rate after infusion of a stresscopin-like peptide with SEQ ID NO:1 for healthy dogs, healthy subjects, and heart-failure subjects.

### DETAILED DESCRIPTION OF THE INVENTION

This invention relates to novel peptides that are selective CHRH2 agonists and compositions thereof for the treatment, amelioration or inhibition of cardiovascular conditions, including but not limited to heart failure. In one embodiment, the novel and selective CRHR2 agonist peptides include stresscopin-like peptides and modifications thereof.

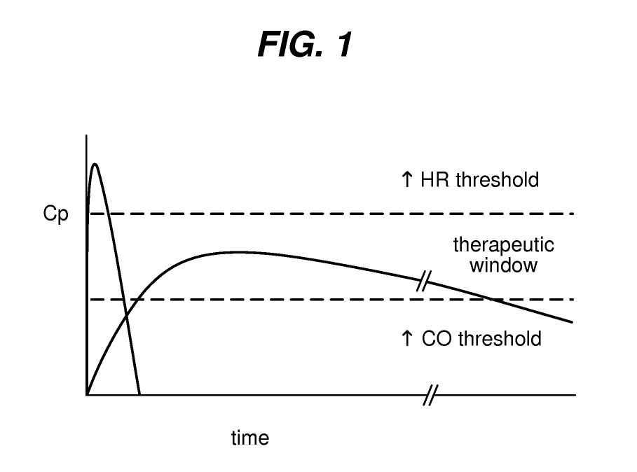

Another embodiment of this invention concerns the administration of stresscopin-like peptides to a patient in need of treatment for heart failure targeting a specific therapeutic blood plasma level range of the administered peptides (FIG. 1). Administration of stresscopin-like peptides in this range improves cardiac performance in the patient without negatively affecting the heart. Such negative effects can include among others any of the following effects: increased heart rate, increased or decreased blood pressure, increased myocardial oxygen consumption, de novo ventricular arrhythmia, and other chronotropic or inotropic responses that significantly stress the failing heart.

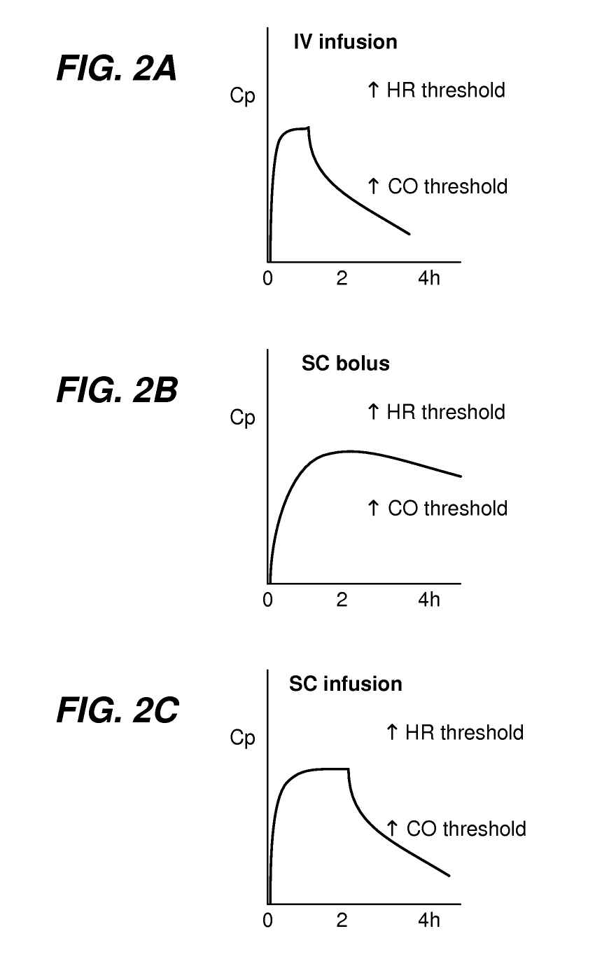

Yet another embodiment of the invention is directed to stresscopin-like peptides and methods of administering them that result in prolonged time intervals, during which their blood plasma level is maintained inside that therapeutically beneficial range (FIGS. 2A-C), and preferably yields a substantially flat plasma curve.

In an embodiment of the invention, a method of treating or ameliorating heart failure in a subject in need thereof comprises administering to the subject a therapeutically effective amount of at least one stresscopin-like peptide in such a way so that the blood plasma concentration of the peptide is substantially maintained below 7.2 ng/mL.

In specific embodiments, the stresscopin-like peptide is selected from a group consisting of stresscopin (h-SCP) and modifications thereof. The stresscopin-like peptide, or modifications thereof, is preferably a mammalian peptide, specifically, a mouse, rat, guinea pig, rabbit, dog, cat, horse, cow, pig, or primate peptide, or derivative thereof. Preferably, the peptide is a human peptide, or derivative thereof.

Modification of a stresscopin-like peptide as used in this invention comprises a change to the amino acid sequence of the compound at at least one position in the amino acid sequence, including amino acid insertions, deletions, and substitutions. Preferably, a modified stresscopin-like peptide binds to the CRH receptor type 2 in a similar way as the unmodified peptide and thus displays at least some physiological activity. Examples of stresscopin-like peptides and modifications thereof are described in more detail in the section below.

Another embodiment of the invention comprises a reactive group covalently attached to a stresscopin-like peptide. The reactive group is chosen for its ability to form a stable covalent bond with a polymer or other chemical moiety that extends the circulation half-life of the peptide in the subject. In an embodiment, such a polymer comprises a polyethylene gycol (PEG) polymer that prolongs the duration of the peptide in the subject's circulation before its elimination. In this form the reactive group is acting as linker between the peptide by reacting on one hand with one or more amino acids of the peptide and on the other with the polymer. In an alternative embodiment, the reactive group is initially bound to the PEG before forming a chemical bond with peptide. In a preferred embodiment of the modified peptides, the linker group is a succinimide, more particular an N-ethylsuccinimide, or an acetamide. Furthermore, the linker may be vinyl sulphone or orthopyridyl disulfide. Preferably, chemical modifications are performed on isolated peptides, e.g. to increase the reaction efficiencies.

Linkers that are useful to bind the polypeptide and the PEG moiety would convey minimal immunogenicity and toxicity to the host. Examples of such linkers may be found in Bailon et al., PSTT, 1998, vol. 1(8), pp. 352-356 or Roberts et al., 2002, Adv. Drug Del. Rev., vol. 54, pp. 459-476. Examples of suitable chemical moieties, in particular PEGs and equivalent polymers, are described in Greenwald et al., 2003, Adv. Drug Del. Rev., vol. 55, pp. 217-250. For example, styrene-maleic anhydride neocarzinostatin copolymer, hydroxylpropyl methacrylamide copolymer, dextran, polyglutamic acid, hydroxylethyl starch, and polyaspartic acid are other polymeric systems that can be employed to accomplish delivery and pharmacokinetic characterics similar to a PEG system.

In certain embodiments of the invention, the stresscopin-like peptide contains an amidated C-terminus. Such modification procedures may be performed on an isolated purified polypeptide or, as in the case of solid-phase synthesis, may be performed during the synthesis procedure. Such procedures are reviewed in Ray et al., Nature Biotech., 1993, vol. 11, pp. 64-70; Cottingham et al., Nature Biotech., 2001, vol. 19, pp. 974-977; Walsh et al., Nature Biotech., vol. 24, pp. 1241-1252; and U.S. Pat. Pub. No. 2008/0167231.

In a particular embodiment of the invention, the compound comprises a stresscopin-like peptide of an amino acid sequence as set forth in SEQ ID NO:82 or in SEQ ID NO:102 containing a CONH2 at its carboxy terminus and a linker bound to the cysteine residue at position 28 of the amino acid sequence with the linker being N-ethylsuccinimide or acetamide, and the linker attached to a PEG polymer of about 20 kDa.

One embodiment of the present invention features dosing compounds comprising stresscopin-like peptides as a method of administering such stresscopin-like peptide to treat heart failure patients.

Furthermore, one embodiment of the present invention features a method of treating a subject suffering or diagnosed with a disease, disorder or condition mediated by CHRH2 activity comprising administering to the subject a therapeutically effective amount of at least one stresscopin-like peptide.

Another embodiment of the present invention features a method for treating or inhibiting the progression of one or more CHRH2-mediated conditions, diseases, or disorders, said method comprising administering to a patient in need of treatment a pharmaceutically effective amount of at least one stresscopin-like peptide.

### A) Terms

The present invention is best understood by reference to the following definitions, the drawings and exemplary disclosure provided herein.

The following are abbreviations that are at times used in this specification: pA₅₀ or pEC₅₀=negative logarithm base 10 of the agonist concentration required to produce half maximum effect; SEM=standard error of the mean; Log DR=logarithm base 10 of the agonist dose ratio; MW=molecular weight; cAMP=adenosine 3′,5′-cyclic monophosphate; cDNA=complementary DNA; kb=kilobase (1000 base pairs); kDa=kilodalton; ATP=adenosine 5′-triphosphate; nt=nucleotide; bp=base pair; PAGE=polyacrylamide gel electrophoresis; PCR=polymerase chain reaction, nm=nanomolar.

The terms “comprising”, “containing”, and “including,” are used herein in their open, non-limiting sense.

“Administering” or “administration” means providing a drug to a patient in a manner that is pharmacologically useful.

“Area under the curve” or “AUC” is the area as measured under a plasma drug concentration curve. Often, the AUC is specified in terms of the time interval across which the plasma drug concentration curve is being integrated, for instance AUC_{start-finish}. Thus, AUC_{0-48 h} refers to the AUC obtained from integrating the plasma concentration curve over a period of zero to 48 hours, where zero is conventionally the time of administration of the drug or dosage form comprising the drug to a patient. AUCₜ refers to area under the plasma concentration curve from hour 0 to the last detectable concentration at time t, calculated by the trapezoidal rule. AUC_{inf} or AUC_{0-∞} refers to the AUC value extrapolated to infinity, calculated as the sum of AUCₜ and the area extrapolated to infinity, calculated by the concentration at time t (Cₜ) divided by k.

“Blood pressure” (BP) is the pressure (force per unit area) exerted by circulating blood on the walls of blood vessels. The pressure of the circulating blood decreases as it moves away from the heart through arteries and capillaries, and toward the heart through veins. Generally, the term blood pressure refers to brachial arterial pressure, which is the blood pressure in the major blood vessel of the upper left or right arm that takes blood away from the heart. For each heartbeat, blood pressure varies between systolic and diastolic pressures. Systolic pressure is peak pressure in the arteries, which occurs near the end of the cardiac cycle when the ventricles are contracting. Diastolic pressure is minimum pressure in the arteries, which occurs near the beginning of the cardiac cycle when the ventricles are filled with blood. An example of normal measured values for a resting, healthy adult human is 115 mmHg systolic and 75 mmHg diastolic. Pulse pressure is the difference between systolic and diastolic pressures. Systolic and diastolic arterial blood pressures are not static but undergo natural variations from one heartbeat to another and throughout the day in response to stress, nutritional factors, drugs, disease, exercise, and momentarily from standing up.

“C” or “Cp” means the concentration of drug in blood plasma, or serum, of a subject, generally expressed as mass per unit volume, typically nanograms per milliliter (ng/mL). For convenience, this concentration may be referred to herein as “drug plasma concentration”, “plasma drug concentration”, “blood plasma concentration” or “plasma concentration”. The plasma drug concentration at any time following drug administration is referenced as Cₜ, as in C_{9 h} or C_{24 h}, etc. A maximum plasma concentration obtained following administration of a dosage form obtained directly from the experimental data without interpolation is referred to as Cₘₐₓ, wherein “tₘₐₓ” is the time elapsed from administration of a dosage form to a subject until the time, at which Cₘₐₓ occurs. The average or mean plasma concentration obtained during a period of interest is referred to as C_{avg} or Cₘₑₐₙ. Persons of skill in the art will appreciate that blood plasma drug concentrations obtained in individual subjects will vary due to interpatient variability in the many parameters affecting drug absorption, distribution, metabolism and excretion. For this reason, unless otherwise indicated, when a drug plasma concentration is listed, the value listed is the calculated mean value based on values obtained from a groups of subjects tested or from multiple administrations to the same subject on different occasions.

Furthermore, a person skilled in the art will appreciate the variability in measured blood plasma concentration of peptides due to the assay utilized in the determination of the peptide quantity, i.e. sandwich immunoassay. The variability can be for instance due to the antibody utilized and is generally normalized across multiple analytic methods based on comparison to reference standards. In light of this assay dependency, someone skilled in the art will accordingly adjust concentration values with regard to underlying assay when comparing concentrations obtained from different assays.

“Substantially maintained” or “substantially maintaining” a level of blood plasma concentration refers to limiting maximal fluctuations of the concentration value to about 10% over a time period larger than about 15 minutes. Fluctuations of the concentration value are measured with regard to a time-averaged concentration value that is averaged over at least 1 to 2 hours. In addition, substantially maintaining a level of blood plasma concentration below a specified upper limit refers to limiting the time period that the concentration value exceeds the upper limit to a time period preferably of less than 15 minutes, more preferably where the time period is less than 10 minutes.

“Cardiac performance” entails overall physiological actions carried out by the heart. Increased cardiac performance includes positive physiological effects on the performance of the heart, while effects negatively influencing the heart's actions are said to decrease the cardiac performance. Such negative effects can include among others any of the following effects: increased heart rate, increased blood pressure, increased myocardial oxygen consumption, de novo ventricular arrhythmia, and other chronotropic or inotropic responses that significantly stress the healthy or failing heart. Furthermore, occurrence of tachyphylaxis is not beneficial to cardiac performance. Increased or improved cardiac performance can be measured by increased ejection fraction, more specifically left ventricular (LV) ejection fraction (EF), larger stroke volume (SV), increased cardiac output (CO), improved systolic and diastolic function, particularly LV function, beneficial chronotropic and inotropic responses, steady or marginally decreased heart rate, steady or decreased blood pressure, i.e. peak systolic aortic pressure, LV end diastolic pressure, LV pressure during isovolumic relaxation or contraction, mean pulmonary artery wedge pressure, in addition to constant or decreased myocardial oxygen consumption, and generally hemodynamic responses beneficial to the overall well-being of the subject.

“Composition” means a product containing a compound of the present invention (such as a product comprising the specified ingredients in the specified amounts, as well as any product which results, directly or indirectly, from such combinations of the specified ingredients in the specified amounts).

“Compound” or “drug” means stresscopin-like peptide or pharmaceutically acceptable forms thereof. “Conjugate” means a chemical compound that has been formed by the joining of two or more compounds.

“Dosage” means administration of a therapeutic agent in prescribed amounts.

“Dosage form” means one or more compounds in a medium, carrier, vehicle, or device suitable for administration to a patient. “Oral dosage form” means a dosage form suitable for oral administration. If not otherwise stated a dosage refers to a dosage form suitable for administration of a dose via the parenteral route. Preferably, the dosage is delivered through continuously intravenuous, or subcutaneous administration.

“Dose” means a unit of drug. Conventionally, a dose is provided as a dosage form. Doses may be administered to patients according to a variety of dosing regimens or dosing rates. Common dosing regimens include once daily (qd), twice daily (bid), thrice daily (tid), four-times daily (qid), twice-a-week, biweekly or monthly. Common dosing rates for continous intravenuous administration include nanograms per dosing minutes and per patient weight in kilograms, where the dose is continuously delivered for at least about 30 minutes, commonly up to a few hours. Common dose amounts for bolus intravenuous or subcutaneous administration include microgram per patient weight in kilogram, generally administered by injection.

“Flat plasma curve” means a plasma concentration curve that reaches and maintains a substantially constant value after a defined period of time following administration of a dosage form according to the invention. The concentration range of constant value is referred to as the “target” plasma concentration.

“Forms” means various isomers and mixtures of one or more stresscopin-like peptides. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers). The term “stereoisomer” refers to isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry.

“Heart rate” (HR) means the number of heartbeats per unit of time, usually expressed as beats per minute (bpm). The average resting human heart rate is about 70 bpm for adult males and 75 bpm for adult females. Heart rate varies significantly between individuals based on fitness, age and genetics. Endurance athletes often have very low resting heart rates. Heart rate can be measured by monitoring one's pulse. An increase of more than 5-10 bpm from the baseline HR of a resting individual for more than about 15 min substantiates a “substantial increase” in HR.

“Parenteral route” means a route of administration that involves piercing the skin or mucous membrane, and generally includes intravenous (IV), subcutaneous (SC), intramuscular (IM) route of administration.

“Patient” or “subject” means an animal, preferably a mammal, more preferably a human, in need of therapeutic intervention.

“Pharmaceutically acceptable” means molecular entities and compositions that are of sufficient purity and quality for use in the formulation of a composition or medicament of the present invention. Since both human use (clinical and over-the-counter) and veterinary use are equally included within the scope of the present invention, a formulation would include a composition or medicament for either human or veterinary use.

“Pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

“Pharmaceutically acceptable salt” means an acid or base salt of the compounds of the invention that is of sufficient purity and quality for use in the formulation of a composition or medicament of the present invention and are tolerated and sufficiently non-toxic to be used in a pharmaceutical preparation. Suitable pharmaceutically acceptable salts include acid addition salts which may, for example, be formed by reacting the drug compound with a suitable pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

“Plasma drug concentration curve”, “drug plasma concentration curve”, “plasma concentration curve”, “plasma concentration-time profiles”, “plasma concentration profile”, or “plasma profile” refer to the curve obtained by plotting plasma drug concentration or drug plasma concentration, or plasma concentration versus time. Usually, the convention is that the zero point on the time scale (conventionally on the x axis) is the time of administration of the drug or dosage form comprising the drug to a patient.

“Rate” means to the quantity of compound administered from a dosage form per unit time, e.g., nanograms of drug delivered per weight of a patient and per minute (ng/kg/min) into the blood circulation of the patient. Drug delivery rates for dosage forms may be measured as an in vitro rate of drug delivery, i.e., a quantity of drug delivered from the dosage form per unit weight and per unit time measured under appropriate conditions and in a suitable fluid. Delivering an amount of drug into the blood circulation of a patient is interchangeably used for administering an equivalent amount of drug.

“Stresscopin-like peptide” means a polypeptide homologous in its amino acid sequence of SEQ ID NO:1 or a derivative of the polypeptide, which includes but is not limited to h-SCP and conservative amino acid substitutions in the sequence of the polypeptide. A homologous stresscopin-like peptide refers to a peptide that comprises an amino acid sequence identical to the h-SCP (SEQ ID NO:1) except for up to but not more than 4 amino acid deletions and/or one or more conservative amino acid substitution. Conserative substitutions may be made, for example, according to the following: aliphatic non-polar, polar-uncharged, and polar charged amino acids can be substituted for another aliphatic amino acid that is non-polar, polar-unchargeed, or polar-charged amino acid, respectively. Preferably, aliphatic non-polar substitutions occur between amino acids in the group consisting of G, A, and P or between amino acids in the group consisting of I, L, and V. Preferably, aliphatic polar-uncharged substitutions occur between amino acids in the group consisting of C, S, T, and M or between amino acids in the group consisting of N and Q. Preferably, aliphatic polar-charged substitutions occur between amino acids in the group consisting of D and E or between amino acids in the group consisting of K and R. Conservative amino acid substitutions can also be made between aromatic amino acids that include H, F, W and Y. Preferably, at least a portion of the homologous stresscopin-like peptide comprises an amino acid sequence with a 90% sequence identity to h-SCP concerning amino acid deletions and/or non-conservative substitutions.

Generally, a stresscopin-like peptide refers to a peptide that displays an agonistic activity towards human corticotrophin releasing hormone receptor type 1 (CRHR1) and type 2 (CRHR2) closely resembling the CRHR1 and CRHR2 activity of stresscopin (h-SCP). A stresscopin-like peptide is a selective CRHR2 agonist with less activity towards CRHR1. Selectivity towards a receptor hereby refers to the potency of a peptide to induce an activity response in the receptor that the peptide is selective towards in comparison to other receptors, in which the peptide might also induce activity, but with less potency. The definition of stresscopin-like peptides is not limited to agonist, but can also include partial agonists. The CRHR1 and CRHR2 activity of a stresscopin-like peptide can for instance be assessed in an adenosine 3′,5′-cyclic monophosphate (cAMP) assay.

By “stresscopin-relative” concentration of a peptide or derivative thereof is meant the concentration that is weight and CRHR2 activity equivalent to a concentration amount of the stresscopin peptide of SEQ ID NO:1. As the molecular weight and CRHR2 activity is different for various forms of stresscopin-like peptides, it is confusing to report the blood plasma concentration for a dosage form without considering the weight or the CRHR2 activity of the peptide. It is preferred to report the blood plasma concentration of a peptide as the stresscopin-relative concentration that is the concentration of the peptide normalized with regard to the weight and CRHR2 activity equivalent to stresscopin. For instance the molecular weight of a pegylated derivative of a stresscopin-like peptide (SEQ ID NO:102) is 25,449 Da, while the molecular weight of stresscopin (SEQ ID NO:1) is 4,367 Da. Furthermore, the agonistic activity of stresscopin-like peptide of SEQ ID NO:102 possesses a pA₅₀ value of 8.15 measured in a CRHR2 cAMP assay versus a pA₅₀ value of 9.40 for stresscopin of SEQ ID NO:1. Hence the agonist potency ratio of a peptide of SEQ ID NO:102 to stresscopin of SEQ ID NO:1 is reduced by a factor of 10^{(9.40-8.15)}=17.78, while the peptide has a 5.6-fold higher mass than stresscopin of SEQ ID NO:1. To dose to a blood plasma level equivalent of 100 pg/mL of stresscopin of SEQ ID NO:1, one should dose to a blood plasma concentration of the stresscopin-like peptide of SEQ ID NO:102 that is 100.8 (=5.6*17.78) times higher, namely 10 ng/mL, assuming equal distribution to tissues from plasma. In case the concentration is quoted in molar units, which are weight independent, one should administer a dose of the stresscopin-like peptide of SEQ ID NO:102 that is 5.6 times higher than the concentration of stresscopin of SEQ ID NO:1 to achieve a pharmacological equivalence based on CRHR2 activity. In summary, the stresscopin-relative concentration of 100 pg/mL of a peptide of SEQ ID NO:102 is equivalent to a concentration of 10 ng/mL of the same peptide. A “stresscopin-relative” dosing rate is one that is based upon achieving a “stresscopin-relative” concentration.

“Terminal half-life” (t_{1/2} or t_{1/2 terminal}) is the time required to reach half the plasma concentration of the pseudo-equilibrium state, a state in which the plasma curve is flat, between drug absorption and drug clearance. When the process of absorption is not a limiting factor, half-life is a hybrid parameter controlled by plasma clearance and extent of distribution. In contrast, when the process of absorption is a limiting factor, the terminal half-life reflects rate and extent of absorption and is independent of the elimination process. The terminal half-life is especially relevant to multiple dosing regimens, because it controls the degree of drug accumulation, concentration fluctuations and the time taken to reach equilibrium.

“Therapeutically effective amount” means that amount of compound that elicits the biological or medicinal response in a tissue system, animal or human, that is being sought by a researcher, veterinarian, medical doctor, or other clinician, which includes therapeutic alleviation of the symptoms of the disease or disorder being treated.

The term “treating” as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating.

### B) Compounds

The present invention relates to the following peptides and derivatives thereof. In general, the invention relates to all compounds that upon administration to patients in need of treatment of heart failure improve cardiac performance in the patient without negatively affecting the heart. Improvement can be measured by increased cardiac output and ejection fraction, while negative effects can include increased heart rate, increased myocardial oxygen consumption, decreased blood pressure among other responses that stress the failing heart. Compounds of the present invention also include novel and selective CRHR2 agonist peptides including stresscopin-like peptides and modifications thereof.

Furthermore, compounds of the present invention refer to chemical or peptidic moieties that bind to or complex with CRHR2, such as h-SCP or mimetic h-SCP polypeptides. Preferred compounds are peptides that have an increased agonistic activity towards CRHR2 as for example measured in a cAMP assay with a pA₅₀ that is within the range of about 7.5 and higher, or pK_{I} (negative log of K_{I}) that is within the range of about 7.5 and higher. Besides displaying high binding affinity, stresscopin-like peptides are CRHR2 agonists that show an elevated level of receptor activation. Peptides that are homologous to h-SCP are therefore preferable, since these peptides naturally possess similar physical and chemical properties.

Members of the family of Corticotropin Releasing Factors exhibit a moderately short half-life. CRHR2 selective agonists promise a unique therapeutic profile. For the treatment of disorders that are mediated by CRHR2, including but not limited to, cardiovascular and metabolic disease, one embodiment of this invention is directed to a long acting variant of stresscopin-like peptides. A long acting stresscopin-like peptide provides particular benefits for the treatment of chronic disorders where the need for continued therapeutic exposure and patient compliance with prescribed treatment are a challenge.

Accordingly, one embodiment of the current invention is directed in general to sequence variation(s) of h-SCP, site specific sequence variations, and spatial or steric interference considerations such that the desired therapeutic profile and/or structure-activity relationship relative to CRHR2 is retained.

Embodiments of stresscopin-like peptides, which are amidated at the C-termini, are provided in Tables 1 through 5. The reactive group or linker is preferably succinimide or acetamide. The modified peptides optionally contain a PEG group. The PEG may vary in length and weight, and is preferably about 20 kDa. Optionally, the number of reactive groups can be more than one, with one reactive group being preferable.

TABLE 1
Human stresscopin with amidated C-terminus and
Cys-variant stresscopin-like peptides

| | |
|---|---|
| TKFTL SLDVP TNIMN LLFNI AKAKN LRAQA AANAH LMAQI-NH₂ | SEQ ID NO: 1 |
| CKFTL SLDVP TNIMN LLFNI AKAKN LRAQA AANAH LMAQI-NH₂ | SEQ ID NO: 2 |
| TCFTL SLDVP TNIMN LLFNI AKAKN LRAQA AANAH LMAQI-NH₂ | SEQ ID NO: 3 |
| TKCTL SLDVP TNIMN LLFNI AKAKN LRAQA AANAH LMAQI-NH₂ | SEQ ID NO: 4 |
| TKFCL SLDVP TNIMN LLFNI AKAKN LRAQA AANAH LMAQI-NH₂ | SEQ ID NO: 5 |
| TKFTC SLDVP TNIMN LLFNI AKAKN LRAQA AANAH LMAQI-NH₂ | SEQ ID NO: 6 |
| TKFTL CLDVP TNIMN LLFNI AKAKN LRAQA AANAH LMAQI-NH₂ | SEQ ID NO: 7 |
| TKFTL SCDVP TNIMN LLFNI AKAKN LRAQA AANAH LMAQI-NH₂ | SEQ ID NO: 8 |
| TKFTL SLCVP TNIMN LLFNI AKAKN LRAQA AANAH LMAQI-NH₂ | SEQ ID NO: 9 |
| TKFTL SLDCP TNIMN LLFNI AKAKN LRAQA AANAH LMAQI-NH₂ | SEQ ID NO: 10 |
| TKFTL SLDVC TNIMN LLFNI AKAKN LRAQA AANAH LMAQI-NH₂ | SEQ ID NO: 11 |
| TKFTL SLDVP CNIMN LLFNI AKAKN LRAQA AANAH LMAQI-NH₂ | SEQ ID NO: 12 |
| TKFTL SLDVP TCIMN LLFNI AKAKN LRAQA AANAH LMAQI-NH₂ | SEQ ID NO: 13 |
| TKFTL SLDVP TNCMN LLFNI AKAKN LRAQA AANAH LMAQI-NH₂ | SEQ ID NO: 14 |
| TKFTL SLDVP TNICN LLFNI AKAKN LRAQA AANAH LMAQI-NH₂ | SEQ ID NO: 15 |
| TKFTL SLDVP TNIMC LLFNI AKAKN LRAQA AANAH LMAQI-NH₂ | SEQ ID NO: 16 |
| TKFTL SLDVP TNIMN CLFNI AKAKN LRAQA AANAH LMAQI-NH₂ | SEQ ID NO: 17 |
| TKFTL SLDVP TNIMN LCFNI AKAKN LRAQA AANAH LMAQI-NH₂ | SEQ ID NO: 18 |
| TKFTL SLDVP TNIMN LLCNI AKAKN LRAQA AANAH LMAQI-NH₂ | SEQ ID NO: 19 |
| TKFTL SLDVP TNIMN LLFCI AKAKN LRAQA AANAH LMAQI-NH₂ | SEQ ID NO: 20 |
| TKFTL SLDVP TNIMN LLFNC AKAKN LRAQA AANAH LMAQI-NH₂ | SEQ ID NO: 21 |
| TKFTL SLDVP TNIMN LLFNI CKAKN LRAQA AANAH LMAQI-NH₂ | SEQ ID NO: 22 |
| TKFTL SLDVP TNIMN LLFNI ACAKN LRAQA AANAH LMAQI-NH₂ | SEQ ID NO: 23 |
| TKFTL SLDVP TNIMN LLFNI AKCKN LRAQA AANAH LMAQI-NH₂ | SEQ ID NO: 24 |
| TKFTL SLDVP TNIMN LLFNI AKACN LRAQA AANAH LMAQI-NH₂ | SEQ ID NO: 25 |
| TKFTL SLDVP TNIMN LLFNI AKAKC LRAQA AANAH LMAQI-NH₂ | SEQ ID NO: 26 |
| TKFTL SLDVP TNIMN LLFNI AKAKN CRAQA AANAH LMAQI-NH₂ | SEQ ID NO: 27 |
| TKFTL SLDVP TNIMN LLFNI AKAKN LCAQA AANAH LMAQI-NH₂ | SEQ ID NO: 28 |
| TKFTL SLDVP TNIMN LLFNI AKAKN LRCQA AANAH LMAQI-NH₂ | SEQ ID NO: 29 |
| TKFTL SLDVP TNIMN LLFNI AKAKN LRACA AANAH LMAQI-NH₂ | SEQ ID NO: 30 |
| TKFTL SLDVP TNIMN LLFNI AKAKN LRAQC AANAH LMAQI-NH₂ | SEQ ID NO: 31 |
| TKFTL SLDVP TNIMN LLFNI AKAKN LRAQA CANAH LMAQI-NH₂ | SEQ ID NO: 32 |
| TKFTL SLDVP TNIMN LLFNI AKAKN LRAQA ACNAH LMAQI-NH₂ | SEQ ID NO: 33 |
| TKFTL SLDVP TNIMN LLFNI AKAKN LRAQA AACAH LMAQI-NH₂ | SEQ ID NO: 34 |
| TKFTL SLDVP TNIMN LLFNI AKAKN LRAQA AANCH LMAQI-NH₂ | SEQ ID NO: 35 |
| TKFTL SLDVP TNIMN LLFNI AKAKN LRAQA AANAC LMAQI-NH₂ | SEQ ID NO: 36 |
| TKFTL SLDVP TNIMN LLFNI AKAKN LRAQA AANAH CMAQI-NH₂ | SEQ ID NO: 37 |
| TKFTL SLDVP TNIMN LLFNI AKAKN LRAQA AANAH LCAQI-NH₂ | SEQ ID NO: 38 |
| TKFTL SLDVP TNIMN LLFNI AKAKN LRAQA AANAH LMCQI-NH₂ | SEQ ID NO: 39 |
| TKFTL SLDVP TNIMN LLFNI AKAKN LRAQA AANAH LMACI-NH₂ | SEQ ID NO: 40 |
| TKFTL SLDVP TNIMN LLFNI AKAKN LRAQA AANAH LMAQC-NH₂ | SEQ ID NO: 41 |

TABLE 2
Cys-variant of stresscopin peptide with
N-Ethylsuccinimide (NES) reactive group

| | |
|---|---|
| TKFTL SLDVP TNIMN LLFNI AKAKN LRAQA AANAH LMAQC(-NES)-NH₂ | SEQ ID NO: 42 |
| TKFTL SLDVP TNIMN LLFNI AKAKN LRAQA AANAC(-NES) LMAQI-NH₂ | SEQ ID NO: 43 |
| TKFTL SLDVP TNIMN LLFNI AKAKN LRAQA AAC(-NES)AH LMAQI-NH₂ | SEQ ID NO: 44 |
| TKFTL SLDVP TNIMN LLFNI AKAKN LRAQA AC(-NES)NAH LMAQI-NH₂ | SEQ ID NO: 45 |
| TKFTL SLDVP TNIMN LLFNI AKAKN LRAQA C(-NES)ANAH LMAQI-NH₂ | SEQ ID NO: 46 |
| TKFTL SLDVP TNIMN LLFNI AKAKN LRC(-NES)QA AANAH LMAQI-NH₂ | SEQ ID NO: 47 |
| TKFTL SLDVP TNIMN LLFNI AKAKN C(-NES)RAQA AANAH LMAQI-NH₂ | SEQ ID NO: 48 |
| TKFTL SLDVP TNIMN LLFNI AKAKC(-NES) LRAQA AANAH LMAQI-NH₂ | SEQ ID NO: 49 |
| TKFTL SLDVP TNIMN LLFNI AKAC(-NES)N LRAQA AANAH LMAQI-NH₂ | SEQ ID NO: 50 |
| TKFTL SLDVP TNIMN LLFNC(-NES) AKAKN LRAQA AANAH LMAQI-NH₂ | SEQ ID NO: 51 |
| TKFTL SLDVP TNIMN LLFC(-NES)I AKAKN LRAQA AANAH LMAQI-NH₂ | SEQ ID NO: 52 |
| TKFTL SLDVP TNIMN LC(-NES)FNI AKAKN LRAQA AANAH LMAQI-NH₂ | SEQ ID NO: 53 |
| TKFTL SLDVP TNIMN C(-NES)LFNI AKAKN LRAQA AANAH LMAQI-NH₂ | SEQ ID NO: 54 |

TABLE 3
Pegylated Cys-variant stresscopin-like peptides with N-
Ethylsuccinimide (NES) linker and PEG weighing about 20 kDa

| | |
|---|---|
| C(-NES-PEG)KFTL SLDVP TNIMN LLFNI AKAKN LRAQA AANAH LMAQI-NH2 | SEQ ID NO: 55 |
| TC(-NES-PEG)FTL SLDVP TNIMN LLFNI AKAKN LRAQA AANAH LMAQI-NH2 | SEQ ID NO: 56 |
| TKC(-NES-PEG)TL SLDVP TNIMN LLFNI AKAKN LRAQA AANAH LMAQI-NH2 | SEQ ID NO: 57 |
| TKFC(-NES-PEG)L SLDVP TNIMN LLFNI AKAKN LRAQA AANAH LMAQI-NH2 | SEQ ID NO: 58 |
| TKFTC(-NES-PEG) SLDVP TNIMN LLFNI AKAKN LRAQA AANAH LMAQI-NH2 | SEQ ID NO: 59 |
| TKFTL C(-NES-PEG)LDVP TNIMN LLFNI AKAKN LRAQA AANAH LMAQI-NH2 | SEQ ID NO: 60 |
| TKFTL SC(-NES-PEG)DVP TNIMN LLFNI AKAKN LRAQA AANAH LMAQI-NH2 | SEQ ID NO: 61 |
| TKFTL SLC(-NES-PEG)VP TNIMN LLFNI AKAKN LRAQA AANAH LMAQI-NH2 | SEQ ID NO: 62 |
| TKFTL SLDC(-NES-PEG)P TNIMN LLFNI AKAKN LRAQA AANAH LMAQI-NH2 | SEQ ID NO: 63 |
| TKFTL SLDVC(-NES-PEG) TNIMN LLFNI AKAKN LRAQA AANAH LMAQI-NH2 | SEQ ID NO: 64 |
| TKFTL SLDVP C(-NES-PEG)NIMN LLFNI AKAKN LRAQA AANAH LMAQI-NH2 | SEQ ID NO: 65 |
| TKFTL SLDVP TC(-NES-PEG)IMN LLFNI AKAKN LRAQA AANAH LMAQI-NH2 | SEQ ID NO: 66 |
| TKFTL SLDVP TNC(-NES-PEG)MN LLFNI AKAKN LRAQA AANAH LMAQI-NH2 | SEQ ID NO: 67 |
| TKFTL SLDVP TNIC(-NES-PEG)N LLFNI AKAKN LRAQA AANAH LMAQI-NH2 | SEQ ID NO: 68 |
| TKFTL SLDVP TNIMC(-NES-PEG) LLFNI AKAKN LRAQA AANAH LMAQI-NH2 | SEQ ID NO: 69 |
| TKFTL SLDVP TNIMN C(-NES-PEG)LFNI AKAKN LRAQA AANAH LMAQI-NH2 | SEQ ID NO: 70 |
| TKFTL SLDVP TNIMN LC(-NES-PEG)FNI AKAKN LRAQA AANAH LMAQI-NH2 | SEQ ID NO: 71 |
| TKFTL SLDVP TNIMN LLC(-NES-PEG)NI AKAKN LRAQA AANAH LMAQI-NH2 | SEQ ID NO: 72 |
| TKFTL SLDVP TNIMN LLFC(-NES-PEG)I AKAKN LRAQA AANAH LMAQI-NH2 | SEQ ID NO: 73 |
| TKFTL SLDVP TNIMN LLFNC(-NES-PEG) AKAKN LRAQA AANAH LMAQI-NH2 | SEQ ID NO: 74 |
| TKFTL SLDVP TNIMN LLFNI C(-NES-PEG)KAKN LRAQA AANAH LMAQI-NH2 | SEQ ID NO: 75 |
| TKFTL SLDVP TNIMN LLFNI AC(-NES-PEG)AKN LRAQA AANAH LMAQI-NH2 | SEQ ID NO: 76 |
| TKFTL SLDVP TNIMN LLFNI AKC(-NES-PEG)KN LRAQA AANAH LMAQI-NH2 | SEQ ID NO: 77 |
| TKFTL SLDVP TNIMN LLFNI AKAC(-NES-PEG)N LRAQA AANAH LMAQI-NH2 | SEQ ID NO: 78 |
| TKFTL SLDVP TNIMN LLFNI AKAKC(-NES-PEG) LRAQA AANAH LMAQI-NH2 | SEQ ID NO: 79 |
| TKFTL SLDVP TNIMN LLFNI AKAKN C(-NES-PEG)RAQA AANAH LMAQI-NH2 | SEQ ID NO: 80 |
| TKFTL SLDVP TNIMN LLFNI AKAKN LC(-NES-PEG)AQA AANAH LMAQI-NH2 | SEQ ID NO: 81 |
| TKFTL SLDVP TNIMN LLFNI AKAKN LRC(-NES-PEG)QA AANAH LMAQI-NH2 | SEQ ID NO: 82 |
| TKFTL SLDVP TNIMN LLFNI AKAKN LRAC(-NES-PEG)A AANAH LMAQI-NH2 | SEQ ID NO: 83 |
| TKFTL SLDVP TNIMN LLFNI AKAKN LRAQC(-NES-PEG) AANAH LMAQI-NH2 | SEQ ID NO: 84 |
| TKFTL SLDVP TNIMN LLFNI AKAKN LRAQA C(-NES-PEG)ANAH LMAQI-NH2 | SEQ ID NO: 85 |
| TKFTL SLDVP TNIMN LLFNI AKAKN LRAQA AC(-NES-PEG)NAH LMAQI-NH2 | SEQ ID NO: 86 |
| TKFTL SLDVP TNIMN LLFNI AKAKN LRAQA AAC(-NES-PEG)AH LMAQI-NH2 | SEQ ID NO: 87 |
| TKFTL SLDVP TNIMN LLFNI AKAKN LRAQA AANC(-NES-PEG)H LMAQI-NH2 | SEQ ID NO: 88 |
| TKFTL SLDVP TNIMN LLFNI AKAKN LRAQA AANAC(-NES-PEG) LMAQI-NH2 | SEQ ID NO: 89 |
| TKFTL SLDVP TNIMN LLFNI AKAKN LRAQA AANAH C(-NES-PEG)MAQI-NH2 | SEQ ID NO: 90 |
| TKFTL SLDVP TNIMN LLFNI AKAKN LRAQA AANAH LC(-NES-PEG)AQI-NH2 | SEQ ID NO: 91 |
| TKFTL SLDVP TNIMN LLFNI AKAKN LRAQA AANAH LMC(-NES-PEG)QI-NH2 | SEQ ID NO: 92 |
| TKFTL SLDVP TNIMN LLFNI AKAKN LRAQA AANAH LMAC(-NES-PEG)I-NH2 | SEQ ID NO: 93 |
| TKFTL SLDVP TNIMN LLFNI AKAKN LRAQA AANAH LMAQC(-NES-PEG)-NH2 | SEQ ID NO: 94 |

TABLE 4
Pegylated Cys-variant stresscopin-like
peptides with PEGs of variable molar weight and
N-Ethylsuccinimide (NES) or Acetamide (IA) linker

| | |
|---|---|
| TKFTL SLDVP TNIMN LLFNI AKAKN LRC(-NES-PEG MW2000)QA | SEQ ID NO: 95 |
| AANAH LMAQI-NH₂ | |
| TKFTL SLDVP TNIMN LLFNI AKAKN LRC(-NES-PEG MW5000)QA | SEQ ID NO: 96 |
| AANAH LMAQI-NH₂ | |
| TKFTL SLDVP TNIMN LLFNI AKAKN LRC(-NES-PEG MW12000)QA | SEQ ID NO: 97 |
| AANAH LMAQI-NH₂ | |
| TKFTL SLDVP TNIMN LLFNI AKAKN LRC(-NES-PEG MW20000)QA | SEQ ID NO: 82 |
| AANAH LMAQI-NH₂ | |
| TKFTL SLDVP TNIMN LLFNI AKAKN LRC(-NES-PEG MW20000 & | SEQ ID NO: 98 |
| DOUBLE-ENDED)QA AANAH LMAQI-NH₂ | |
| TKFTL SLDVP TNIMN LLFNI AKAKN LRC(-NES-PEG MW30000)QA | SEQ ID NO: 99 |
| AANAH LMAQI-NH₂ | |
| TKFTL SLDVP TNIMN LLFNI AKAKN LRC(-NES-PEG MW40000)QA | SEQ ID NO: 100 |
| AANAH LMAQI-NH₂ | |
| TKFTL SLDVP TNIMN LLFNI AKAKN LRC(-NES-PEG MW80000 & | SEQ ID NO: 101 |
| BRANCHED)QA AANAH LMAQI-NH₂ | |
| TKFTL SLDVP TNIMN LLFNI AKAKN LRC(-IA-PEG MW20000)QA | SEQ ID NO: 102 |
| AANAH LMAQI-NH₂ | |
| TKFTL SLDVP TNIMN LLFNI AKAKN LRC(-IA-PEG MW30000)QA | SEQ ID NO: 103 |
| AANAH LMAQI-NH₂ | |
| TKFTL SLDVP TNIMN LLFNI AKAKN LRC(-IA-PEG MW40000)QA | SEQ ID NO: 104 |
| AANAH LMAQI-NH₂ | |
| TKFTL SLDVP TC(-IA-PEG MW20000)IMN LLFNI AKAKN LRAQA | SEQ ID NO: 105 |
| AANAH LMAQI-NH₂ | |
| TKFTL SLDVP TNIMN LLFNI AKAKN LRAQA AANAC(-IA-PEG | SEQ ID NO: 106 |
| MW20000) LMAQI-NH₂ | |

TABLE 5
Stresscopin-like peptides with shortened amino acid (aa)
sequence compared to peptide of SEQ ID NO: 1

| | | |
|---|---|---|
| TKFTL SLDVP TNIMN LLFNI AKAKN LRAQA AANAH LMAQI-NH₂ | 40 aa | SEQ ID NO: 1 |
| KFTLS LDVPT NIMNL LFNIA KAKNL RAQAA ANAHL MAQI-NH₂ | 39 aa | SEQ ID NO: 107 |
| TLSLD VPTNI MNLLF NIAKA KNLRA QAAAN AHLMA QI-NH₂ | 37 aa | SEQ ID NO: 108 |
| LSLDV PTNIM NLLFN IAKAK NLRAQ AAANA HLMAQ I-NH₂ | 36 aa | SEQ ID NO: 109 |
| SLDVP TNIMN LLFNI AKAKN LRAQA AANAH LMAQI-NH₂ | 35 aa | SEQ ID NO: 110 |
| LDVPT NIMNL LFNIA KAKNL RAQAA ANAHL MAQI-NH₂ | 34 aa | SEQ ID NO: 111 |
| DVPTN IMNLL FNIAK AKNLR AQAAA NAHLM AQI-NH₂ | 33 aa | SEQ ID NO: 112 |
| FTLSL DVPTN IMNLL FNIAK AKNLR AQAAA NAHLM AQI-NH₂ | h-UCN3 | SEQ ID NO: 116 |

Drug compounds of the present invention also include a mixture of stereoisomers, or each pure or substantially pure isomer. For example, the present compound may optionally have one or more asymmetric centers at a carbon atom containing any one substituent. Therefore, the compound may exist in the form of enantiomer or diastereomer, or a mixture thereof. When the present compound contains a double bond, the present compound may exist in the form of geometric isomerism (cis-compound, trans-compound), and when the present compound contains an unsaturated bond such as carbonyl, then the present compound may exist in the form of a tautomer, and the present compound also includes these isomers or a mixture thereof. The starting compound in the form of a racemic mixture, enantiomer or diastereomer may be used in the processes for preparing the present compound. When the present compound is obtained in the form of a diastereomer or enantiomer, they can be separated by a conventional method such as chromatography or fractional crystallization. In addition, the present compound includes an intramolecular salt, hydrate, solvate or polymorphism thereof.

Furthermore, suitable drug compounds are those that exert a local physiological effect, or a systemic effect, either after penetrating the mucosa or—in the case of oral administration—after transport to the gastrointestinal tract with saliva. The dosage forms prepared from the formulations according to the present invention are particularly suitable for drug compounds that exert their activity during an extended period of time, in particular drugs that have a half-life of at least several hours.

### C) Synthesis Routes & Purification

An “isolated” polypeptide is a polypeptide substantially free of or separated from cellular material or other contaminating proteins from the cell or tissue source from which the polypeptide is produced and isolated, or substantially free of chemical precursors or other chemicals when the polypeptide is chemically synthesized. For example, protein that is substantially free of cellular material can include preparations of protein having less than about 30%, or preferably 20%, or more preferably 10%, or even more preferably 5%, or yet more preferably 1% (by dry weight), of contaminating proteins.

### Biological Route

In preferred embodiments, the isolated polypeptide is substantially pure. Thus, when the polypeptide is recombinantly produced, it is substantially free of culture medium, e.g., culture medium representing less than about 20%, or more preferably 10%, or even more preferably 5%, or yet more preferably 1%, of the volume of the protein preparation. When the protein is produced by chemical synthesis, it is substantially free of chemical precursors or other chemicals, i.e., it is separated from chemical precursors or other chemicals that are involved in the synthesis of the protein. Accordingly such preparations of the polypeptide have less than about 30%, or preferably 20%, or more preferably 10%, or even more preferably 5%, or yet more preferably 1% (by dry weight), of chemical precursors or compounds other than the polypeptide of interest.

Polypeptide expression in cellular environments may be achieved by the utilization of isolated polynucleotides. An “isolated” polynucleotide is one that is substantially separated from or free of nucleic acid molecules with differing nucleic acid sequences. Embodiments of isolated polynucleotide molecules include cDNA, genomic DNA, RNA, and anti-sense RNA. Preferred polynucleotides are obtained from biological samples derived from a human, such as from tissue specimens.

Vectors may be used to deliver and propagate polynucleotides encoding the polypeptide. Introduction of such vectors into host cells may yield production of the encoded mRNA or protein of the mimetic stresscopin. Alternatively, expression vectors may be combined with purified elements including but not limited to transcription factors, RNA polymerase, ribosomes, and amino acids to produce efficient transcription/translation reactions in cell free conditions. Mimetic stresscopin polypeptides expressed from the resulting reactions may be isolated for further purification, modification, and/or formulation.

The term vector refers to a nucleic acid molecule capable of transporting another nucleic acid to which it has been linked. An exemplary type of vector is a plasmid, which refers to a circular double-stranded DNA loop into which additional DNA segments can be inserted. Another example of a vector is a viral vector wherein additional DNA segments can be inserted. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors-expression vectors—are capable of directing the expression of genes to which they are operably linked. Vectors of utility in recombinant DNA techniques may be in the form of plasmids. Alternatively, other forms of vectors, such as viral vectors (e.g. replication defective retroviruses, adenoviruses and adeno-associated viruses), which serve equivalent functions, may be selected by the artisan as suitable for the intended use.

A host cell refers to a cell that contains a DNA molecule either on a vector or integrated into a cell chromosome. A host cell can be either a native host cell that contains the DNA molecule endogenously or a recombinant host cell. One example of a host cell is a recombinant host cell, which is a cell that has been transformed or transfected by an exogenous DNA sequence. A cell has been transformed by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA. A clone refers to a population of cells derived from a single cell or common ancestor by mitosis. A cell line refers to a clone of a primary cell that is capable of stable growth in vitro for many generations. Recombinant host cells may be prokaryotic or eukaryotic, including bacteria such as E. coli, fungal cells such as yeast, mammalian cells such as cell lines of human, bovine, porcine, monkey and rodent origin, and insect cells such as Drosophila and silkworm derived cell lines. A recombinant host cell refers not only to the particular subject cell, but also to the progeny or potential progeny of such a cell. Particularly because certain modifications can occur in succeeding generations due to either mutation or environmental influences, such progeny may not be identical to the parent cell, but are still intended to be included within the scope of the term.

Illustrative vectors of the present invention also include specifically designed expression systems that allow the shuttling of DNA between hosts, such as bacteria-yeast or bacteria-animal cells or bacteria-fungal cells or bacteria-invertebrate cells. Numerous cloning vectors are known to those skilled in the art and the selection of an appropriate cloning vector is within the purview of the artisan. For other suitable expression systems for both prokaryotic and eukaryotic cells see, e.g., chapters 16 and 17 of Sambrook et al., (1989), MOLECULAR CLONING: A LABORATORY MANUAL, vol. 2, pp. 16.3-16.81.

In order to obtain high level expression of a cloned gene or nucleic acid, such as a cDNA encoding a mimetic stresscopin polypeptide, a nucleotide sequence corresponding to the mimetic stresscopin polypeptide sequence is preferably subcloned into an expression vector that contains a strong promoter to direct transcription, a transcription/translation terminator, and if for a nucleic acid encoding a protein, a ribosome binding site for translational initiation. Suitable bacterial promoters are known in the art and are described, e.g., by Sambrook et al., (1989), MOLECULAR CLONING: A LABORATORY MANUAL, Second Edition, Cold Spring Harbor Laboratory, Cold Spring Harbor, N.Y. and Makrides, 1996, Microbiol. Rev. 60(3):512-38. Bacterial expression systems for expressing the mimetic stresscopin proteins disclosed in the present invention are available in, e.g., E. coli, Bacillus sp., and Salmonella (Palva et al., 1983, Gene, 22:229-235; Mosbach et al., 1983, Nature, 302:543-545). Kits for such expression systems are commercially available. Eukaryotic expression systems for mammalian cells, yeast, and insect cells are known in the art and are also commercially available. In exemplary embodiments, the eukaryotic expression vector is a baculovirus vector, adenoviral vector, an adeno-associated vector, or a retroviral vector.

A promoter refers to a regulatory sequence of DNA that is involved in the binding of RNA polymerase to initiate transcription of a gene. Promoters are often upstream (i.e., 5′) to the transcription initiation site of the gene. A gene refers to a segment of DNA involved in producing a peptide, polypeptide, or protein, including the coding region, non-coding regions preceding (5′UTR) and following (3′UTR) coding region, as well as intervening non-coding sequences (introns) between individual coding segments (exons). Coding refers to the specification of particular amino acids or termination signals in three-base triplets (codons) of DNA or mRNA.

The promoter used to direct expression of the polynucleotide may be routinely selected to suit the particular application. The promoter is optionally positioned about the same distance from the heterologous transcription start site as it is from the transcription start site in its natural setting. As will be apparent to the artisan, however, some variation in this distance can be accommodated without loss of promoter function.

In addition to the promoter, the expression vector may contain a transcription unit or expression cassette that contains all the additional elements required for the expression of the mimetic stresscopin -encoding polynucleotide in host cells. An exemplary expression cassette contains a promoter operably linked to the polynucleotide sequence encoding a mimetic stresscopin polypeptide, and signals required for efficient polyadenylation of the transcript, ribosome binding sites, and translation termination. The polynucleotide sequence encoding a canine mimetic stresscopin polypeptide may be linked to a cleavable signal peptide sequence to promote secretion of the encoded protein by the transfected cell. Exemplary signal peptides include the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence, the human stresscopin gene, or may be obtained from different genes.

In exemplary embodiments, any of the vectors suitable for expression in eukaryotic or prokaryotic cells known in the art may be used. Exemplary bacterial expression vectors include plasmids such as pBR322-based plasmids, pSKF, pET23D, and fusion expression systems such as GST and LacZ. Examples of mammalian expression vectors include, e.g., pCDM8 (Seed, 1987, Nature, 329:840) and pMT2PC (Kaufman et al., 1987, EMBO J., 6:187-193). Commercially available mammalian expression vectors which can be suitable for recombinant expression of polypeptides of the invention include, for example, pMAMneo (Clontech, Mountain View, Calif.), pcDNA4 (Invitrogen, Carlsbad, Calif.), pCiNeo (Promega, Madison, Wis.), pMC1neo (Stratagene, La Jolla, Calif.), pXT1 (Stratagene, La Jolla, Calif.), pSG5 (Stratagene, La Jolla, Calif.), EBO-pSV2-neo (ATCC 37593), pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

Epitope tags may also be added to recombinant proteins to provide convenient methods of isolation, e.g., c- myc, hemoglutinin (HA)-tag, 6-His tag, maltose binding protein, VSV-G tag, or anti-FLAG tag, and others available in the art.

Expression vectors containing regulatory elements from eukaryotic viruses may be used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo 5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification, such as neomycin, thymidine kinase, hygromycin B phosphotransferase, and dihydrofolate reductase. Alternatively, high yield expression systems not involving gene amplification are also suitable, such as using a baculovirus vector in insect cells, with a sequence encoding a mimetic stresscopin polypeptide under the direction of the polyhedrin promoter or other strong baculovirus promoters.

Elements that can be included in expression vectors also include a replicon that functions in E. coli, a gene encoding antibiotic resistance to permit selection of bacteria that harbor recombinant plasmids, and unique restriction sites in nonessential regions of the plasmid to allow controlled insertion of eukaryotic sequences. The particular antibiotic resistance gene may be selected from the many resistance genes known in the art. The prokaryotic sequences may be chosen such that they do not interfere with the replication of the DNA in eukaryotic cells, if necessary or desired.

Known transfection methods may be used to produce bacterial, mammalian, yeast or insect cell lines that express large quantities of a SCP mimetic, which are then purified using standard techniques, such as selective precipitation with such substances as ammonium sulfate, column chromatography, and immunopurification methods.

Transformation of eukaryotic and prokaryotic cells may be performed according to standard techniques (see, e.g., Morrison, 1977, J Bact., 132:349-351; Clark-Curtiss et al., Methods in Enzymology, 101:347-362).

Any of the known procedures suitable for introducing foreign nucleotide sequences into host cells may be used to introduce the expression vector. These include the use of reagents such as Superfect (Qiagen), liposomes, calcium phosphate transfection, polybrene, protoplast fusion, electroporation, microinjection, plasmid vectors, viral vectors, biolistic particle acceleration (the Gene Gun), or any other known methods for introducing cloned genomic DNA, cDNA, synthetic DNA or other foreign genetic material into a host cell (see, e. g., Sambrook et al., supra). The particular genetic engineering procedure selected should be capable of successfully introducing at least one gene into the host cell capable of expressing a mimetic stresscopin RNA, mRNA, cDNA, or gene.

As would be apparent to artisans, for stable transfection of mammalian cells, depending upon the expression vector and transfection technique used, only a small fraction of cells may integrate the foreign DNA into their genome. In order to identify and select these integrants, a gene that encodes a selectable marker (e.g., for resistance to antibiotics) may be introduced into the host cells along with the gene of interest. Exemplary selectable markers include those which confer resistance to drugs, such as G-418, puromycin, geneticin, hygromycin and methotrexate. Cells stably transfected with the introduced nucleic acid can be selected for and identified by drug selection (e.g., cells that have incorporated the selectable marker gene will survive, while the other cells die).

A heterologous regulatory element may be inserted into a stable cell line or cloned microorganism, such that it is operatively linked with and activates expression of endogenous genes, using techniques such as targeted homologous recombination, e.g., as described in U.S. Pat. No. 5,272,071 and International Publication No. WO 91/06667. After the expression vector is introduced into the cells, the transfected cells are preferably cultured under conditions optimally favoring expression of the mimetic stresscopin polypeptide, which is recovered from the culture using standard techniques identified below. Methods of culturing prokaryotic or eukaryotic cells are known in the art; see, e.g., Sambrook et al., supra; Freshney, 1993, CULTURE OF ANIMAL CELLS, 3rd ed.

As an alternative to using cellular systems for polypeptide production, cell-free systems have shown the capability for gene expression and synthesis in prokaryotic (Zubay G., Annu Rev Genet., 1973, 7:267-287) and eukaryotic systems (Pelham et al., Eur J Biochem., 1976, 67:247-256; Anderson et al., Meth Enzymol., 1983, 101:635-644). These systems can utilize either mRNA or DNA nucleotides for polypeptide synthesis reactions. A preferred technique for cell-free polypeptide production uses reticulocyte lysate, RNA polymerase, nucleotides, salts, and ribonuclease inhibitor in one quick coupled transcription/translation reaction (TNT®, Promega, Madison, Wis., U.S.A.).

### Solid-Phase Synthesis

Peptides of the invention may be prepared using the solid-phase synthetic technique initially described by Merrifield, in J. Am. Chem. Soc., 85:2149-2154 (1963). Other peptide synthesis techniques may be found, for example, in M. Bodanszky et al., (1976) Peptide Synthesis, John Wiley & Sons, 2d Ed.; Kent and Clark-Lewis in Synthetic Peptides in Biology and Medicine, p. 295-358, eds. Alitalo, K., et al., Science Publishers, (Amsterdam, 1985); as well as other reference works known to those skilled in the art. A summary of peptide synthesis techniques may be found in Steward et al., Solid Phase Peptide Synthelia, Pierce Chemical Company, Rockford, Ill. (1984), which is incorporated herein by reference. The synthesis of peptides by solution methods may also be used, as described in The Proteins, Vol. II, 3d Ed., p. 105-237, Neurath, H. et al., Eds., Academic Press, New York, N.Y. (1976). Appropriate protective groups for use in such syntheses will be found in the above texts, as well as in J. F. W. McOmie, Protective Groups in Organic Chemistry, Plenum Press, New York, N.Y. (1973), which is incorporated herein by reference. In general, these synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Block synthesis techniques may also be applied to both the solid phase and solution methods of peptide synthesis. Rather than sequential addition of single amino acid residues, preformed blocks comprising two or more amino acid residues in sequence are used as either starting subunits or subsequently added units rather than single amino acid residues.

Using a solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Solid support synthesis may be achieved with automated protein synthesizers (Protemist®, CellFree Sciences, Matsuyama Ehime 790-8577, Japan; Symphony SMPS-110, Rainin, Woburn, Ma., U.S.A.; ABI 433A peptide synthesizer, Applied Biosystems, Foster City, Calif., U.S.A.). Such machines have the capability to perform automated protein reactions that allow for greater control and optimization of the synthesis.

### Purification

A number of procedures may be employed to isolate or purify the inventive polypeptide. For example, column chromatography may be used to purify polypeptides based on their physical properties, i.e. hydrophobicity. Alternatively, proteins having established molecular adhesion properties may be reversibly fused to the inventive polypeptide. With an appropriate ligand for the fused protein, the mimetic stresscopin polypeptide may be selectively adsorbed to a purification column and then freed from the column in a substantially pure form. The fused protein may then be removed by enzymatic activity. Alternative column purification strategies may employ antibodies raised against the mimetic stresscopin polypeptide. These antibodies may be conjugated to column matrices and the polypeptides purified via these immunoaffinity columns.

Recombinant proteins may be separated from the host reactions by suitable separation techniques such as salt fractionation. This method may be used to separate unwanted host cell proteins (or proteins derived from the cell culture media) from the recombinant protein of interest. An exemplary salt is ammonium sulfate, which precipitates proteins by effectively reducing the amount of water in the protein mixture (proteins then precipitate on the basis of their solubility). The more hydrophobic a protein is, the more likely it is to precipitate at lower ammonium sulfate concentrations. An exemplary isolation protocol includes adding saturated ammonium sulfate to a protein solution so that the resultant ammonium sulfate concentration is between 20-30%, to precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed to achieve the desired purity, e.g., through dialysis or diafiltration. Other known methods that rely on solubility of proteins, such as cold ethanol precipitation, may be used to fractionate complex protein mixtures.

In other examples of isolation or purification techniques, the molecular weight of the inventive polypeptide may be used to isolate it from proteins of greater and lesser size using ultrafiltration through membranes of different pore size (for example, Amicon or Millipore membranes). As a first step, the protein mixture is ultra-filtered through a membrane with a pore size that has a lower molecular weight cut-off than the molecular weight of the protein of interest. The retained matter of the ultra-filtration is then ultrafiltered against a membrane with a molecular cut-off greater than the molecular weight of the protein of interest. The recombinant protein will pass through the membrane into the filtrate, and the filtrate may then be chromatographed.

### Chemical Modifications

The inventive polypeptide may be subjected to directed chemical modifications, such as maleimide capping, polyethylene glycol (PEG) attachment, maleidification, acylation, alkylation, esterification, and amidification, to produce structural analogs of the polypeptide. One skilled in the art would appreciate the existence of a variety of chemical modification techniques and moieties, see for example U.S. Pat. Nos. 5,554,728, 6,869,932, 6,828,401, 6,673,580, 6,552,170, 6,420,339, U.S. Pat. Pub. 2006/0210526 and Intl. Pat. App. WO 2006/136586. Preferably, chemical modifications are performed on isolated polypeptide, e.g., to increase reaction efficiencies.

In certain embodiments of the invention, the inventive polypeptide contains an amidated C-terminus. Such polypeptide modification procedures may be performed on isolated purified polypeptide or, as in the case of solid-phase synthesis, may be performed during the synthesis procedure. Such procedures are reviewed in Ray et al., Nature Biotechnology, 1993, vol. 11, pp. 64-70; Cottingham et al., Nature Biotechnology, 2001, vol. 19, pp. 974-977; Walsh et al., Nature Biotechnology, 2006, vol. 24, pp. 1241-1252; U.S. Pat. Appl. Publ. 2008/0167231.

The polypeptides of the invention may contain certain intermediate linkers that are useful to bind the polypeptide and the PEG moiety. Such linkers would convey minimal immunogenicity and toxicity to the host. Examples of such linkers may be found in Bailon et al., PSTT, 1998, vol. 1(8), pp. 352-356.

In certain embodiments, the invention is directed to a conjugate comprising an isolated polypeptide consisting essentially of a sequence as set forth in SEQ ID NO:29 containing a CONH₂ at its carboxy terminus and a intermediate linker conjugated to the cysteine residue at position 28 of the amino acid sequence of SEQ ID NO:29. In certain embodiments, the intermediate linker is N-ethylsuccinimide. In further embodiments the intermediate linker may be vinyl sulphone. In further embodiments, the intermediate linker may be acetamide. In certain embodiments, the intermediate linker may be orthopyridyl disulfide.

In further embodiments, the invention is directed towards a conjugate comprising a polypeptide having the amino acid sequence as set forth in SEQ ID NO:29 with a CONH₂ at its carboxy terminus, an N-ethylsuccinimide linker conjugated to the cysteine residue at position 28 of SEQ ID NO:29, wherein the N-ethylsuccinimide linker is also bound to a PEG moiety. In certain embodiments, the molecular weight of the PEG moiety may range from about 2 kDa to abput 80 kDa. In certain embodiments, the mass of the PEG is about 20 kDa. In preferred embodiments, the stresscopin-like peptide comprises a polypeptide of SEQ ID NO:82 or SEQ ID NO:102. In certain embodiments, the PEG mass is about 5 kDa. In certain other embodiments, the PEG mass is about 12 kDa. In certain embodiments, the PEG mass is about 20 kDa. In certain embodiments, the PEG is mass about 30 kDa. In certain embodiments, the PEG mass is about 40 kDa. In certain embodiments, the PEG mass is about 80 kDa. In certain embodiments, the PEG moiety is linear. In other embodiments, the PEG moiety is branched. PEG moieties may be synthesized according to methods known to one of ordinary skilled in the art. Alternatively, PEG moieties are commercially available, such as SUNBRIGHT® ME-020MA, SUNBRIGHT® ME-050MA, and SUNBRIGHT® ME-200MA (NOF corp., Japan; Sigma Aldrich, St. Louis, Mo., U.S.A.)

The invention further relates to pharmaceutically acceptable salts of the inventive polypeptide and methods of using such salts. A pharmaceutically acceptable salt refers to a salt of a free acid or base of the polypeptide that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A polypeptide may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenylpropionates, phenyl butyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive peptide contains a basic nitrogen, the desired pharmaceutically acceptable salt may be prepared by any suitable method available in the art, for example, treatment of the free base with an inorganic acid, such as hydrochloric acid, hydrobromic acid, hydriodic acid, perchloric acid, sulfuric acid, sulfamic acid, nitric acid, boric acid, phosphoric acid, and the like, or with an organic acid, such as acetic acid, trifluoroacetic acid, phenylacetic acid, propionic acid, stearic acid, lactic acid, ascorbic acid, maleic acid, hydroxymaleic acid, malic acid, pamoic acid, isethionic acid, succinic acid, valeric acid, fumaric acid, saccharinic acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, benzenesulfonic acid, 2-naphthalenesulfonic acid, p-toluenesulfonic acid, methanesulfonic acid, ethanesulfonic acid, hydroxyethanesulfonic, a cyclohexanesulfamic acid, any compatible mixture of acids such as those given as examples herein, and any other acid and mixture thereof that are regarded as equivalents or acceptable substitutes in light of the ordinary level of skill in this technology.

If the inventive polypeptide contains an acid group, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium. Representative organic or inorganic bases further include benzathine, chloroprocaine, choline, diethanolamine, ethylenediamine, meglumine, and procaine.

The invention also relates to pharmaceutically acceptable prodrugs of the compounds, and treatment methods employing such pharmaceutically acceptable prodrugs. The term “prodrug” means a precursor of a designated compound that, following administration to a subject yields the compound in vivo via a chemical or physiological process such as solvolysis or enzymatic cleavage, or under physiological conditions. A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Exemplary prodrugs include compounds having an amino acid residue, or a polypeptide chain of two or more (e.g., two, three or four) amino acid residues, covalently joined through an amide or ester bond to a free amino, hydroxy, or carboxylic acid group of the compound. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of the compound as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl)esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher et al., Adv. Drug Delivery Rev. 1996, 19, 115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)-methyl and (acyloxy)-ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Greenwald, et al., J Med Chem. 1996, 39, 10, 1938-40. Free amines can also be derivatized as amides, sulfonamides or phosphonamides. All of these prodrug moieties may incorporate groups including ether, amine, and carboxylic acid functionalities.

The present invention also relates to pharmaceutically active metabolites of the compounds, which may also be used in the methods of the invention. A “pharmaceutically active metabolite” means a pharmacologically active product of metabolism in the body of the compound or salt thereof. Prodrugs and active metabolites of a compound may be determined using routine techniques known or available in the art. See, e.g., Bertolini, et al., J Med Chem. 1997, 40, 2011-2016; Shan, et al., J Pharm Sci. 1997, 86 (7), 765-767; Bagshawe, Drug Dev Res. 1995, 34, 220-230; Bodor, Adv Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

### D) Pharmaceutical Compositions

In particular embodiments of the invention, stresscopin-like peptides are used alone, or in combination with one or more additional ingredients, to formulate pharmaceutical compositions. A pharmaceutical composition comprises an effective amount of at least one compound in accordance with the invention.

In some embodiments, the pharmaceutical composition comprises a polypeptide having the amino acid sequence as set forth in SEQ ID NO:29, wherein the polypeptide contains a CON H₂ at its carboxy terminus, and further comprises a N-ethylsuccinimide or acetamide linker attached to the cysteine residue at position 28, wherein said linker is also linked to a PEG moiety. PEG moieties are classified by their molecular weight and physical characteristics, such as being linear or branched, and containing one or more linker moieties used to bond the PEG to the polypeptide substrate. In certain preferred embodiments, the polypeptide contains one or two said linkers.

In certain embodiments, the pharmaceutical composition comprising the PEG moiety may contain a PEG moiety whose weight may range from about 2 kDa to about 80 kDa. In certain embodiments, the PEG moiety mass is about 2 kDa. In further embodiments, the PEG mass is about 5 kDa. In certain embodiments, the PEG mass is about 12 kDa. In certain embodiments, the PEG mass is about 20 kDa. In certain embodiments, the PEG mass is about 30 kDa. In certain embodiments, the PEG mass is about 40 kDa. In certain embodiments, the PEG mass is about 80 kDa. Such compositions may further comprise a pharmaceutically acceptable excipient.

The disclosure also provides compositions (including pharmaceutical compositions) comprising a compound or derivatives described herein, and one or more of pharmaceutically acceptable carrier, excipient, and diluent. In certain embodiments of the invention, a composition may also contain minor amounts of wetting or emulsifying agents, or pH buffering agents. In a specific embodiment, the pharmaceutical composition is pharmaceutically acceptable for administration to a human. In certain embodiments, the pharmaceutical composition comprises a therapeutically or prophylactically effective amount of a compound or derivative described herein. The amount of a compound or derivative of the invention that will be therapeutically or prophylactically effective can be determined by standard clinical techniques. Exemplary effective amounts are described in more detail in below sections. In certain embodiments of the invention, a composition may also contain a stabilizer. A stabilizer is a compound that reduces the rate of chemical degradation of the modified peptide of the composition. Suitable stabilizers include, but are not limited to, antioxidants, such as ascorbic acid, pH buffers, or salt buffers.

The pharmaceutical compositions can be in any form suitable for administration to a subject, preferably a human subject. In certain embodiments, the compositions are in the form of solutions, suspensions, emulsion, tablets, pills, capsules, powders, and sustained-release formulations. The compositions may also be in particular unit dosage forms. Examples of unit dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

In a specific embodiment, the subject is a mammal such as a cow, horse, sheep, pig, fowl, cat, dog, mouse, rat, rabbit, or guinea pig. In a preferred embodiment, the subject is a human. Preferably, the pharmaceutical composition is suitable for veterinary and/or human administration. In accordance with this embodiment, the term “pharmaceutically acceptable” means approved by a regulatory agency of the Federal or a state government or listed in the U.S. Pharmacopeia or other generally recognized pharmacopeia for use in animals, and more particularly for use in humans.

Suitable pharmaceutical carriers for use in the compositions are sterile liquids, such as water and oils, including those of petroleum, animal, vegetable or synthetic origin. In a specific embodiment, the oil is peanut oil, soybean oil, mineral oil, or sesame oil. Water is a preferred carrier when the pharmaceutical composition is administered intravenously. Saline solutions and aqueous dextrose and glycerol solutions can also be employed as liquid carriers, particularly for injectable solutions. Further examples of suitable pharmaceutical carriers are known in the art, e.g., as described in Remington's Pharmaceutical Sciences (1990) 18th ed. (Mack Publishing, Easton Pa.).

Suitable excipients for use in the compositions include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. Whether a particular excipient is suitable for incorporation into a pharmaceutical composition depends on a variety of factors well known in the art including, but not limited to, the route of administration and the specific active ingredients in the composition.

In certain embodiments of the invention, a composition is an anhydrous composition. Anhydrous compositions can be prepared using anhydrous or low moisture containing ingredients and low moisture or low humidity conditions. Compositions comprising modified peptides having a primary or secondary amine are preferably anhydrous if substantial contact with moisture and/or humidity during manufacturing, packaging, and/or storage is expected. An anhydrous composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Pharmaceutical compositions comprising the compounds or derivatives described herein, or their pharmaceutically acceptable salts and solvates, are formulated to be compatible with the intended route of administration. The formulations are preferably for subcutaneous administration, but can be for administration by other means such as by inhalation or insufflation (either through the mouth or the nose), intradermal, oral, buccal, parenteral, vaginal, or rectal. Preferably, the compositions are also formulated to provide increased chemical stability of the compound during storage and transportation. The formulations may be lyophilized or liquid formulations.

In one embodiment, the compounds or derivatives are formulated for intravenous administration. Intravenous formulations can include standard carriers such as saline solutions. In another embodiment, the compounds or derivatives are formulated for injection. In a preferred embodiment, the compounds or derivatives are sterile lyophilized formulations, substantially free of contaminating cellular material, chemicals, virus, or toxins. In a particular embodiment, the compounds or derivatives are formulated in liquid form. In another particular embodiment, formulations for injection are provided in sterile single dosage containers. In a particular embodiment, formulations for injection are provided in sterile single dosage containers. The formulations may or may not contain an added preservative. Liquid formulations may take such forms as suspensions, solutions or emulsions in oily or aqueous vehicles, and may contain formulation agents such as suspending, stabilizing and/or dispersing agents.

### E) Administration

A compound or derivative described herein, or a pharmaceutically acceptable salt thereof, is preferably administered as a component of a composition that optionally comprises a pharmaceutically acceptable vehicle. The compound or derivative is preferably administered subcutaneously. Another preferred method of administration is via intravenous injection or continuous intravenous infusion of the compound or derivative. Preferably, the administration is through infusion reaching a pseudo-static steady state in blood plasma levels by slow systemic absorption and clearance of the compound or derivative.

In certain embodiments, the compound or derivative is administered by any other convenient route, for example, by infusion or bolus injection, or by absorption through epithelial or mucocutaneous linings (e.g., oral mucosa, rectal, and intestinal mucosa). Methods of administration include but are not limited to parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In most instances, administration will result in the release of the compound or derivative into the bloodstream. In preferred embodiments, the compound or derivative is delivered intravenously or subcutaneously.

The preparation may be in the form of tablets, capsules, sachets, dragees, powders, granules, lozenges, powders for reconstitution, liquid preparations, or suppositories. Preferably, the compositions are formulated for intravenous infusion or bolus injection, subcutaneous infusion or bolus injection, or intra muscular injection.

The compound is preferably administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution, dextrose solution, and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may be given over a period ranging from several minutes to several days. In yet another embodiment, an effective amount of the inventive peptide may be coated on nanoparticles or provided in a “depot” suitable for subcutaneous delivery (Hawkins et al., Adv Drug Deliv Rev., 2008, vol. 60, pp. 876-885; Montalvo et al., Nanotechnology, 2008, vol. 19, pp. 1-7).

Active agents may be administered through inhalation methods. Such methods may use dry powder (Johnson et al., Adv Drug Del Rev., 1997, vol. 26(1), pp. 3-15) and/or aerosol (Sangwan et al., J Aerosol Med., 2001, vol. 14(2), pp. 185-195; Int. Pat. Appl. WO2002/094342) formulation techniques.

In embodiments of treatment methods according to the invention, a therapeutically effective amount of at least one active agent according to the invention is administered to a subject suffering from or diagnosed as having such a disease, disorder, or condition, such as heart failure, diabetes, skeletal muscle wasting, and sarcopenia. Additional conditions include improper motor activity, food intake, or a need for cardioprotective, bronchorelaxant, and/or anti-inflammatory activity. Therapeutically effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

An exemplary intravenous dose rate is in the range from about 0.2 ng to about 52 ng of stresscopin-relative active agent per kg of subject's body weight per minute, preferably about 0.2 ng/kg/min to about 22 ng/kg/min, or equivalently about 0.3 μg/kg to about 32 μg/kg daily. In the case of bolus infusion or subcutaneous injection, the total dose can be administered in single or divided dosage units (e.g., BID, TID, QID, twice-a-week, biweekly or monthly). For a 70-kg human, an illustrative range for a suitable dosage amount is from about 1 μg/day to about 1 mg/day. Weekly dosage regiments can be used as an alternate to daily administration.

In another preferred embodiment, the CRHR2 peptide agonist of SEQ ID NO:102, which comprises an acetamide linker binding a PEG of about 20 kDa to the cysteine residue at position 28 of the peptide sequence, is administered at a dose of 10 μg/kg by bolus subcutaneous injection to a patient in need thereof. The frequency of this dosage should range from once a day to less frequent based upon the therapeutic needs of the subject and other clinical considerations.

One skilled in the art would use information from models, clinical trials, and information from routine factors, as discussed above, to determine effective amounts of the drug in order for treatment.

In an embodiment, a compound of SEQ ID NO:1 or a pharmaceutical composition thereof is administered through IV infusion such that a steady state of the blood plasma concentration of the therapeutically active compound is reached after about 1 hour for an intended treatment period of 24 hours. After stopping the administration of the drug the therapeutic effect tailors off in about 30 minute. This embodiment may be suitable for an acute care setting (FIG. 2A).

In another embodiment, a compound of SEQ ID NO:1 or a pharmaceutical composition thereof is administered through SC infusion such that a steady state of blood plasma concentration of the therapeutically active compound is reached in about 4 hours. After stopping the administration of the drug the therapeutic effect tailors off in about 1 hour. This embodiment may be suitable for ambulatory care (FIG. 2B).

In yet another embodiment, a compound of SEQ ID NO:82, SEQ ID NO:102 or a pharmaceutical composition thereof is administered through one or more SC bolus injections over a time period ranging from 1 to 7 days to reach a steady state of blood plasma concentration in about 4-8 hours or more. After stopping the administration of the drug the therapeutic effect tailors off in about 3-5 days reducing the effect of the compound. The advantage of this embodiment is low maintenance on side of the patient and the health care professional and it may be adapted to an ambulatory care setting. A possible rescue treatment in light of an adverse event may involve beta-blockers among other medicaments (FIG. 2C).

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. If symptoms have been alleviated to an appropriate level, treatment may cease. Patients may, however, require intermittent treatment on a long-term basis upon any recurrence of symptoms.

In certain embodiments, the compounds or derivative are administered in combination with one or more other biologically active agents as part of a treatment regimen. In certain embodiments, the compounds or derivatives are administered prior to, concurrently with, or subsequent to the administration of the one or more other biologically active agents. In one embodiment, the one or more other biologically active agents are administered in the same pharmaceutical composition with a compound or derivative described herein. In another embodiment, the one or more other biologically active agents are administered in a separate pharmaceutical composition with a compound or derivative described herein. In accordance with this embodiment, the one or more other biologically active agents may be administered to the subject by the same or different routes of administration as those used to administer the compound or derivative.

In another embodiment, the compound or derivative can be administered with one or more other compound or composition for reducing risk or treating a cardiovascular disease. Compounds or compositions the reduce the risk or treat cardiovascular disease include, but are not limited to, anti-inflammatory agents, anti-thrombotic agents, anti-platelet agents, fibrinolytic agents, thrombolytics, lipid reducing agents, direct thrombin inhibitors, anti-Xa inhibitors, anti-IIa inhibitors, glycoprotein IIb/IIIa receptor inhibitors and direct thrombin inhibitors. Examples of agents that can be administered in combination with the compound or derivatives described herein include bivalirudin, hirudin, hirugen, Angiomax, agatroban, PPACK, thrombin aptamers, aspirin, GPIIb/IIIa inhibitors (e.g., Integrelin), P2Y12 inhibitors, thienopyridine, ticlopidine, and clopidogrel.

In embodiments, the compound is formulated into dosage forms suitable for administration to patients in need thereof. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Parrot et al., J. Pharm. Sci., 61(6), pp. 813-829 (1974); and Hixon et al., Chem. Engineering, pp. 94-103 (1990).

The amount of compound incorporated in the dosage forms of the present invention may generally vary from about 10% to about 90% by weight of the composition depending upon the therapeutic indication and the desired administration period, e.g., every 12 hours, every 24 hours, and the like. Depending on the dose of compound desired to be administered, one or more of the dosage forms can be administered. Depending upon the formulation, the compound will preferably be in the form of an HCl salt or free base form.

Further, this invention also relates to a pharmaceutical composition or a pharmaceutical dosage form as described hereinbefore for use in a method of therapy or diagnosis of the human or non-human animal body.

This invention also relates to a pharmaceutical composition for use in the manufacture of a pharmaceutical dosage form for oral administration to a mammal in need of treatment, characterized in that said dosage form can be administered at any time of the day independently of the food taken in by said mammal.

This invention also relates to a method of therapy or diagnosis of the human or non-human animal body that comprises administering to said body a therapeutically or diagnostically effective dose of a pharmaceutical composition described herein.

This invention also relates to a pharmaceutical package suitable for commercial sale comprising a container, a dosage form as described herein, and associated with said package written matter non-limited as to whether the dosage form can be administered with or without food.

The following formulation examples are illustrative only and are not intended to limit the scope of the inventions in any way.

### EXAMPLES

### F) Example Synthesis

### Synthesis 1: Synthesis and Purification of Polypeptide

The polypeptide of SEQ ID NO:29 was prepared by a solid phase peptide synthesis reaction on a Rainin Symphony Multiple Peptide Synthesizer (Model SMPS-110) using software version 3.3.0. Resin (NovaSyn TGR®, 440 mg, approximately 0.1 mmole, 0.23 mmol/g substitution, Lot No. A33379) used for the synthesis of peptide amides was a composite of polyethylene glycol and polystyrene functionalized with an acid-labile modified Rink amide linker.

Amino acids used in synthesis contained Nα-9-Fluorenylmethoxycarbonyl (Fmoc) protection groups on the C-terminus and the following side-chain protecting groups: Arg(2,2,4,6,7-pentamethyldihydrobenzofuran-5-sulfonyl, pbf), Asp(tertiary butoxy, OtBu), Asn(Trityl, Trt), Gln(Trt), Cys(Trt), His(Trt), Lys(t-Butoxycarbonyl, Boc), Ser(tertiary butyl, tBu) and Thr(tBu).

Coupling reactions were carried out by mixing N-Methylpyrrolidinone (NMP) pre-swollen resin (0.1 mmole), a 5-fold molar excess of Fmoc-amino acid in DMF (2.5 mL) and 5-fold molar excess of hexafluorophosphate (HBTU) with a 10-fold molar excess of N-Methylmorpholine (NMM) in DMF (2.5 mL) were added, then coupled for over 45 minutes. For Fmoc removal, reactions were incubated with a 20% Piperidine/DMF solution for 2 minutes. The solution was then drained and fresh 20% Piperidine/DMF was added and incubated for 18 minutes. Reactions were then washed with NMP and subsequent amino acid additions performed by repeat of coupling steps. For C-terminal coupling to Ile40, Gln39, Asn19, Asn12, and Val9 numbered from the N-terminus, the coupling steps were performed twice.

Peptide cleavage from the resin was performed using a two-hour cleavage program and incubation with 9 mL of a cleavage mixture comprising trifluoroacetic acid (TFA) (100 mL), 1,2-ethanedithiol (EDT) (20.0 mL), phenol (7.5 g), thioanisole (5 mL), triisopropylsilane (TIS) (5 mL) and water (5 mL). The solution of cleaved peptide was transferred to a 50-mL BD polypropylene centrifuge tube, and the peptide was precipitated with cold ethyl ether (40 mL). The mixture was centrifuged, and the ethyl ether was decanted from the peptide. Ethyl ether (40 mL) was added, the mixture was vortexed and centrifuged, and the ethyl ether was decanted. These steps (addition of fresh ethyl ether, vortexing, centrifugation, and decanting) were repeated two additional times. The peptide was dried in vacuo to give 408 mg (92% yield) of the crude product.

Polypeptide purification was performed on a Waters preparative HPLC system (Waters, Mass., U.S.A.). The crude peptide (˜100 mg) was dissolved in 20/30/50 acetic acid/acetonitrile/water containing 0.1% TFA. The material injected onto two Vydac C-18 columns (10 mm, 2.5×25 cm). After the injection, a gradient of 0-45% solvent B (solvent B=80% acetonitrile containing 0.1% TFA) over 5 min and 45-70% solvent B over 60 min with a flow rate of 6 mL/min was utilized to purify the peptide. Fractions were collected and analyzed by analytical RP-HPLC, MALDI-TOF MS, and CE. The most pure fractions were pooled and lyophilized to give 23 mg of product. MALDI-TOF MS yielded molecular weight of the product to equal 4400.5, which is larger than the calculated molecular weight for C₁₉₅H₃₂₆N₅₆O₅₃S₃ of 4399.2 by one hydrogen atom. Lyophilization was made by flash freezing the liquid in an acetone dry ice bath for approximately 30 minutes. After freezing, the product, in an open flask, was covered with filter paper and placed under high vacuum. After 24 hours under high vacuum dried sample was removed from vacuum and storage container sealed for future use.

Synthesis 2: Conjugation of Polypeptide with N-Ethylmaleimide

Site directed N-ethylmaleimide capping on cysteine residues as shown in Scheme 1 was achieved under the conditions as follows.

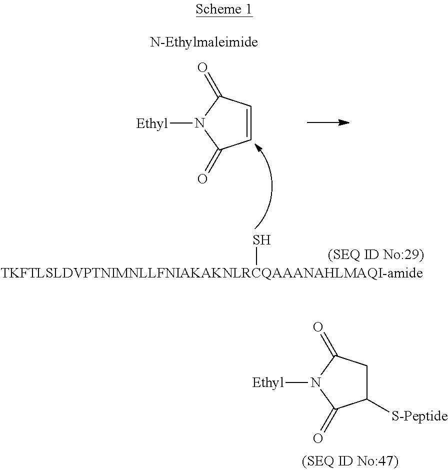

In a 2.5 mL polypropylene vial, 2.0 mg of the inventive peptide was dissolved in 1.0 mL water. Twenty microliters of 0.1 M aqueous N-ethylmaleimide was then added immediately. The reaction was gently agitated at room temperature for 2 hours. The reaction mixtures were purified on a Summit APS (Dionex, Calif., U.S.A.) HPLC fit with a Vydac C18 300 Angstrom, (10×250 mm; Grace Davison, Ill., U.S.A.) column using the following protocol shown in Table 6. End Fractions were collected, analyzed by HPLC, and the pure fractions pooled and lyophilized.

TABLE 6

| Column: | Vydac C18 300 Ångstrom (10 × 250 mm) |
|---|---|
| Solvents: | A: 0.1% TFA in Water |
| | B: 0.1% TFA with 80% Acetonitrile/Water |
| UV: | (1) 214 nm |
| | (2) 280 nm |
| Flow: | 2.000 ml/min at 0.000 min |
| Gradient (% B) at time: | |
| 4.000 min | 0.0% |
| 40.000 min | 100.0% |
| 60.000 min | 100.0% |
| 62.000 min | 0.0% |
| 75.000 min | 0.0% |

Synthesis 3: Conjugation of Polypeptide with Iodoacetamide-PEG

Iodoacetamide-PEG, a linear 20 kDa polyethylene glycol chain with an iodoacetamide terminus, and present in limiting quantities at slightly alkaline pH with polypeptide of SEQ ID NO:29 resulted in cysteine modification as an exclusive reaction as shown in Scheme 2. The cysteine thiol acted as a selective point of attachment for the iodacetamide-PEG. The resulting derivative alpha sulfahydrylacetamide linkage was achiral.

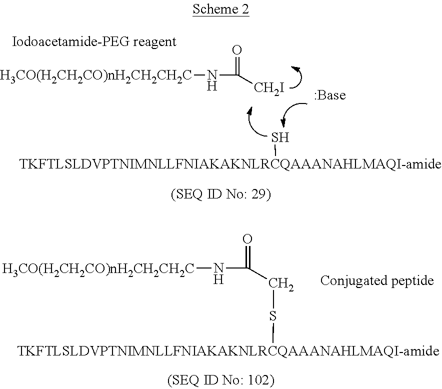

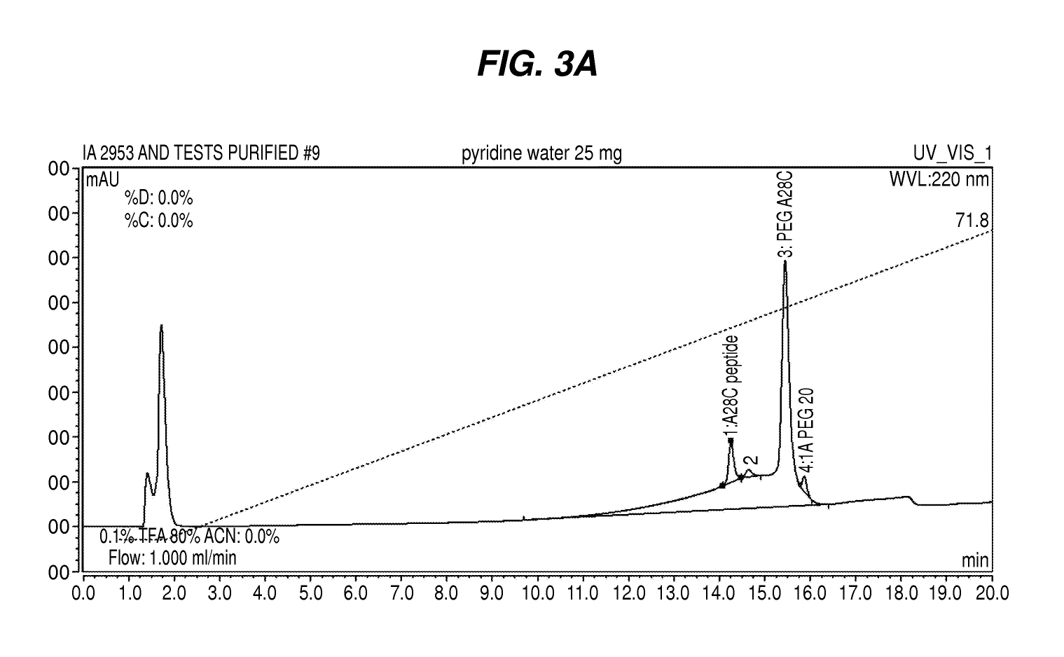

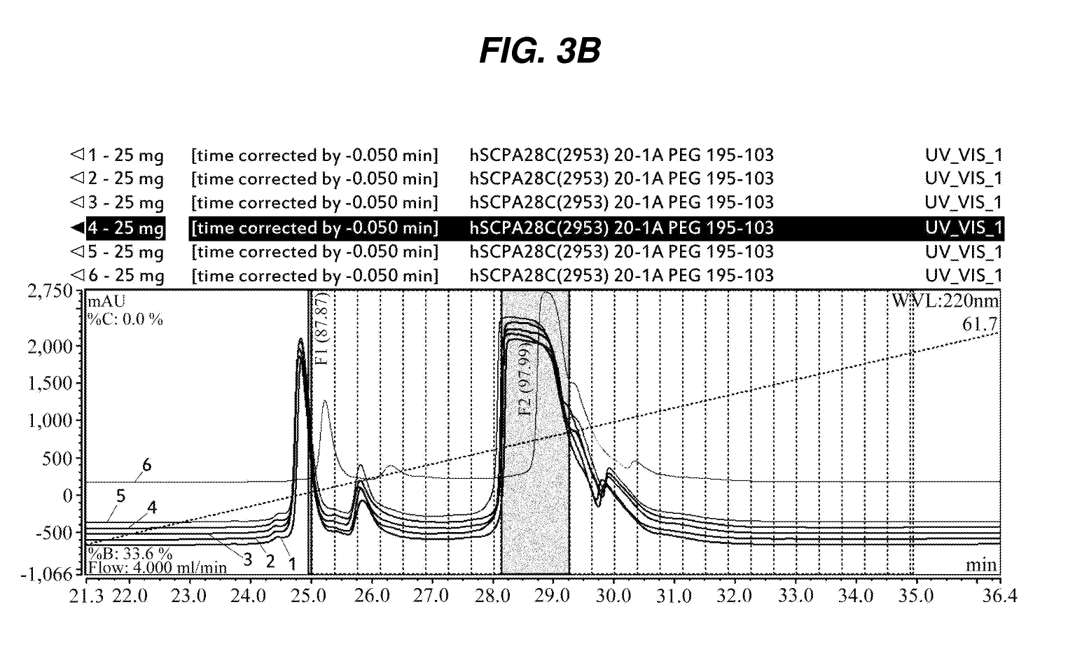

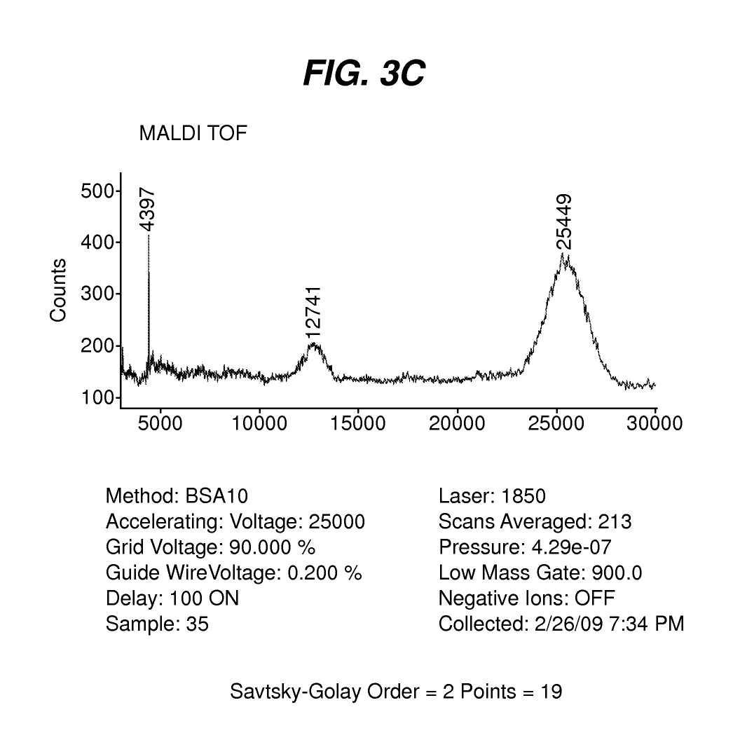

To a 15 mL conical flask, 25 mg (5.68 mmol, 1.0 eq) of peptide of SEQ ID NO:1 was added. Into the same flask 140 mg (6.82 mmol, 1.2 eq, 95% active) PEG-20 iodoacetamide (Lot No. M77592) made by Nippon, Oil and Fat (NOF) Corp. was added. 10 mL of water was added and the solution vortexed until all solids were dissolved. To the cloudy solution, 50 mL of pyridine was added at a solution pH of about 8.91. After 2 hours, a 20 mL aliquot of sample was removed and analyzed by reverse phase HPLC using a Phenomenex C6-phenyl column with 0.1% TFA/acetonitrile as eluents. The sample showed near complete reaction after 2 hours (FIG. 3A). The reaction mixture was purified directly by HPLC using a Phenomenex C6 phenyl 10×150 mm column. Eluents for purification were 0.1% TFA water and 80% acetonitrile in 0.1 TFA water. Purifications were in sample batches of 2-3mL (FIG. 3B). Purified fractions were combined and lyophilized in a 50 mL conical flask. The lyophilized solid was diluted in 10 mL of water and re-lyophilized. Approximately 1 mg of the final product was diluted to 1 mg/mL and submitted for mass spectroscopic analysis (FIG. 3C). The average weight of the pegylated compound of SEQ ID NO:102 was 25,449 Dalton due in part to the heterogeneity in the length of the PEG polymer, and the compound appeared as a white amorphous solid.

Synthesis 4: Pegylation of Polypeptide with N-Ethylmaleimide Linker

In a 2.5 mL polypropylene vial 2.0 mg (˜0.44 nmol) of the polypeptide in was dissolved in 2.5 mL water followed by the immediate addition of activated and N-ethylmaleimide-derivatived polyethylene gycols of varying molecular weight by using the amounts shown in Table 7.

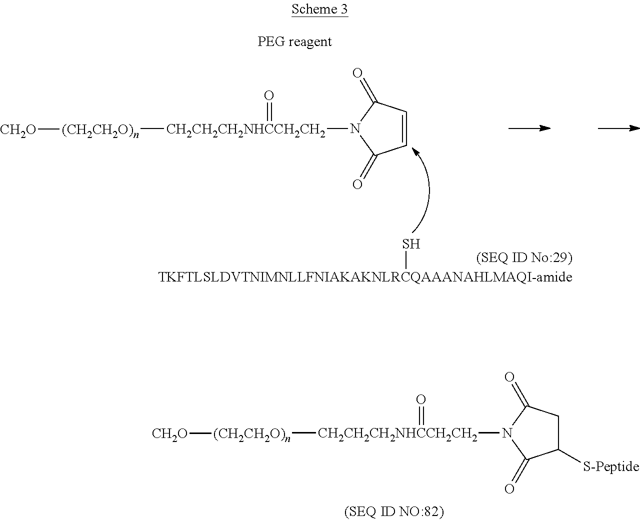

The reaction mixture was gently agitated at room temperature for 2 hours.

TABLE 7

| | PEG-Malemide | | |
|---|---|---|---|
| PEG Structure | MW [kDa] | NOF Corp. Catalog No. | Amount [mg] |
| Linear | 2 | SUNBRIGHT ® ME-020MA | 1.0 mg (0.49 nMol) |
| Linear | 5 | SUNBRIGHT ® ME-050MA | 2.0 mg (0.49 nMol) |
| Linear | 12 | SUNBRIGHT ® ME-120MA | 6.0 mg (0.49 nMol) |
| Linear | 20 | SUNBRIGHT ® ME-200MA | 10.0 mg (0.49 nMol) |
| Linear | 30 | SUNBRIGHT ® ME-300MA | 15.0 mg (0.49 nMol) |
| Linear | 40 | SUNBRIGHT ® ME-400MA | 20.0 mg (0.49 nMol) |
| Branched | 80 | SUNBRIGHT ® GL2-800MA | 40.0 mg (0.49 nMol) |
| Double Ended | 20 | SUNBRIGHT ® DE-200MA | 5.0 mg (0.49 nMol) |
| Maleimide | | | |

The reaction mixtures were purified on a Summit APS (Dionex, Calif., U.S.A.) HPLC fit with a Gemini 5u C6-phenyl 110 Angstrom (10×100 mm; Phenomenex, Calif., U.S.A.) column using the protocol of Table 8.

TABLE 8

| | |
|---|---|
| Column: | Phenomenex Gemini 5u C6-phenyl |
| | 110 Ångstrom (10 × 100 mm) |
| Solvents: | A: 0.1% TFA in Water |
| | B: 0.1% TFA with 80% Acetonitrile/Water |
| UV: | (1) 214 nm |
| | (2) 280 nm |
| Flow: | 4.000 ml/min at 0.000 min |
| Gradient (% B) at time: | |
| 2.500 min | 0.0% |
| 40.000 min | 70.0% |
| 45.000 min | 100.0% |
| 52.000 min | 100.0% |
| 54.000 min | 0.0% |
| 60.000 min | End |

### G) Biological Examples

Study No. 1: CRHR2 and CRHR1 Aqonist Activity—cAMP Assay

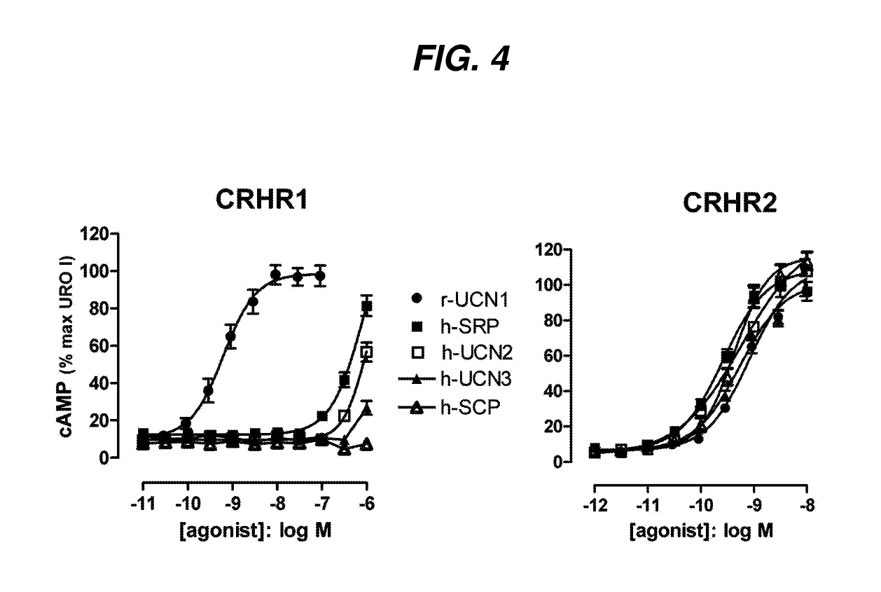

The CRHR2 and CRHR1 agonist activity of the CRH family was characterized in two lines of SK—N-MC (human neuroblastoma) cells transfected with either the human CRHR2 or human CRHR1 in an adenosine 3′,5′-cyclic monophosphate (cAMP) assay. h-SCP (SEQ ID NO:1) was equipotent with h-UCN2 (SEQ ID NO:115) in this assay and shown to be the most selective CRHR2 agonist in the CRH family (FIG. 4). The concentration required for 50% maximum effect (A₅₀) was 0.4 nM.

Human CRHR1 (accession number X72304) or CRHR2 (accession number U34587) were cloned into pcDNA3.1/Zeo expression vector and stably transfected into SK—N-MC cells by electroporation. Cells were maintained in MEM w/Earl's Salt with 10% FBS, 50 I.U. penicillin, 50 μg/ml streptomycin, 2 mM L-glutamine, 1 mM sodium pyruvate and 0.1 mM non-essential amino acids, 600 μg/ml G418. Cells were grown at 37° C. in 5% CO₂

Cells were plated in 96-well tissue culture dishes (Biocoat from BD Biosciences) overnight at 50,000 cell/well. Cells were washed with PBS then resuspended in DMEM F-12 without phenol red, containing 10 μM isobutylmethylxanthine (IBMX). Cells were incubated with the peptides at concentrations ranging from 1 pM to 10 μM for 60 min at 37° Celsius. For subsequent evaluation of any antagonism activity of those peptides that did not produce an agonist response, the peptides were pre-incubated at 10 μM for 20 min prior to the addition of h-SCP for 60 min. Forskolin (10 μM), a direct stimulant of adenylate cyclase, was used as positive control. The assays were stopped by the addition of 0.5 M HCl and mixing by orbital rotation for 2 h at 4° Celsius.

To assess the activity of the inventive polypeptide at the CRHR2, an intracellular cAMP measurement test using a Flash plate radioactive assay (Catalog No. Cus56088; Perkin Elmer, Mass., U.S.A.) was employed.

Transfected SK—N-MC cells were plated in 96-well Biocoat tissue culture dishes (BD Biosciences, San Jose, Calif., U.S.A) overnight at 50,000 cell/well. Cells were first washed with PBS and then suspended with DMEM/F-12 without phenol red, containing μM isobutylmethylxanthine (IBMX). Suspended cells were transferred into a 96-well flash plate coated with scintillant fluid. Cells were incubated with peptides ranging from 1 pM to 1 μM, for 60 min, at 37° Celsius. Forskolin at 10 μM was used as positive control. After ligand stimulation, cells were lysed by the addition of 0.5M HCl and mixed by orbital rotation for 2 h at 4° Celsius in order to release intracellular cAMP into the media.

Media containing released intracellular cAMP was transferred to a 96-well flash plate coated with scintillant fluid containing an anti-cAMP antibody. In this assay, intracellular cAMP competes with ¹²⁵I-labeled cAMP binding to the antibody. To generate a standard curve, cAMP ranging from 2.5 to 250 pmoles/ml was included in the experiment. [¹²⁵I]-cAMP was measured on a TopCount scintillation counter (Perkin Elmer, Mass., U.S.A).

Individual agonist concentration-response curve data were fitted to the Hill equation, see formula below, using GraphPad Prism (Graphpad Software, La Jolla, Calif., U.S.A.), to provide estimates of agonist concentration needed to generate one-half maximal response (A₅₀), and the maximal asymptote (α) and Hill slope (n_{H}) parameters. In this equation, [A] is the agonist concentration and E is the measured effect:

$$E = \frac{α \cdot {[ A ]}^{{n}_{H}}}{{[ A ]}_{50}^{{n}_{H}} + {[ A ]}^{{n}_{H}}}$$

For display purposes the mean fitted parameter estimates were used to generate a single E/[A] curve shown superimposed on the mean experimental data. Potency estimates for agonists, pA₅₀, are expressed as the negative logarithm of the midpoint of each curve and listed with their standard error of measurement (SEM). Logarithm base 10 of the agonist dose ratio (Log DR) values were calculated by subtraction of the test compound pA₅₀ value from the corresponding h-SCP (SEQ ID NO:1) control pA₅₀ value within the same assay batch. The SEM values of the Log DR values are given by the square root of the sum of the squared SEM values of the h-SCP (SEQ ID NO:1) control and test compound pA₅₀ values.

TABLE 9
CRHR antagonist peptide-anti-sauvagine-30

| | | |
|---|---|---|
| FHLLR KMIEI EKQEK EKQQA | SV30 | SEQ ID NO: 118 |
| ANNRL LLDTI-NH₂ | | |

The CRHR2-mediated cAMP response to h-SCP (SEQ ID NO:1) was blocked by the selective CRHR2 antagonist, anti-sauvagine-30 (SV30, SEQ

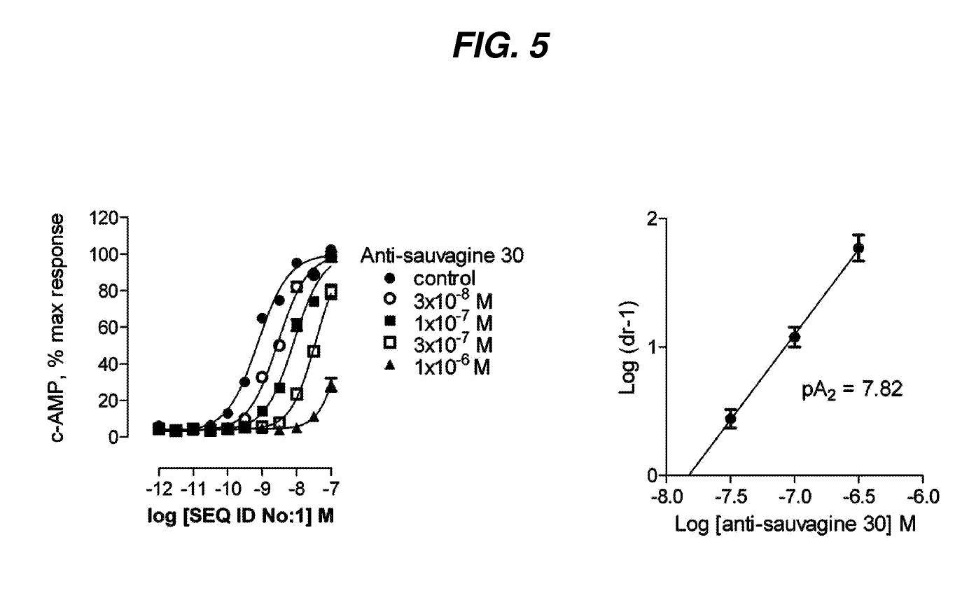

ID NO:118 listed in Table 9), in a concentration-dependent manner consistent with surmountable competitive antagonism (FIG. 5). The presence of anti-sauvagine-30 yielded a pA₂ value of 7.82 for the compound of SEQ ID NO:1.

TABLE 10

| | | |
|---|---|---|
| TKFTL SLDVP TNIMN LLFNI AKAKN LRAQA AANAH LMAQI | non- | SEQ ID NO: 113 |
| | amidated | |
| | h-SCP | |
| DDPPL SIDLT FHLLR TLLEL ARTQS QRERA EQNRI IFDSV-NH₂ | r-UCN1 | SEQ ID NO: 114 |
| IVLSL DVPIG LLQIL LEQAR ARAAR EQATT NARIL ARV-NH₂ | h-UCN2 | SEQ ID NO: 115 |
| HPGSR IVLSL DVPIG LLQIL LEQAR ARAAR EQATT NARIL | h-SRP | SEQ ID NO: 117 |
| ARV-NH₂ | | |

Human and rat peptides (see Table 10) were used on the stimulation of h-CRHR1 or h-CRHR2 transfected SK—N-MC cells in the cAMP flash plate assay. Peptides were incubated for 1 hr at 37° Celsius. Curves were calculated using non-linear regression sigmoidal concentration-response analysis calculation in GraphPad Prism. The so obtained pA₅₀ values are shown in Table 11 in addition to literature values.

TABLE 11

| | | Published | Experimental | | | | | | |
|---|---|---|---|---|---|---|---|---|---|
| Receptor | Peptide | pA₅₀ | pA₅₀ | SEM | n_{H} | SEM | αₘₐₓ | SEM | n |
| CRHR1 | r-UCN1 | 9.82¹ | 9.19 | 0.07 | 1.15 | 0.19 | 99.61 | 3.28 | 12 |
| CRHR1 | h-SRP | >7³ | 6.34 | 0.03 | 1.61 | 0.15 | NA | | 20 |
| CRHR1 | h-SRP | >7³ | 6.2 | 0.04 | 1.33 | 0.17 | NA | | 11 |
| CRHR1 | h-SRP | >7³ | 6.28 | 0.03 | 1.26 | 0.13 | NA | | 17 |
| CRHR1 | h-UCN2 | | 6.02 | 0.02 | 1.69 | 0.18 | NA | | 15 |
| CRHR1 | h-UCN3 | | <5 | | | | | | |
| CRHR1 | h-SCP | | <5 | | | | | | |
| CRHR2 | r-UCN1 | 10.06² | 9.08 | 0.05 | 1.07 | 0.11 | 110.5 | 2.49 | 12 |
| CRHR2 | h-UCN2 | 9.37²/9.12⁵ | 8.04 | 0.05 | 0.9 | 0.09 | 114.7 | 2.89 | 16 |
| CRHR2 | h-UCN3 | 9.92² | 9.26 | 0.05 | 1.02 | 0.11 | 101.8 | 2.18 | 12 |
| CRHR2 | h-SCP | ~9⁴ | 9.41 | 0.06 | 0.99 | 0.12 | 99.31 | 2.69 | 16 |
| CRHR2 | h-SRP | ~9⁴ | 9.32 | 0.05 | 1.08 | 0.11 | 113.5 | 2.3 | 16 |
| CRHR2 | h-SCP | ~9⁴ | 9.15 | 0.03 | 1.04 | 0.06 | 97.53 | 1.29 | 32 |
| CRHR2 | h-SCP | ~9⁴ | 9.36 | 0.04 | 1.39 | 0.05 | 116.1 | 2.59 | 20 |
| CRHR2 | h-SCP | ~9⁴ | 9.39 | 0.02 | 1.55 | 0.12 | 98.2 | 1.31 | 30 |
| CRHR2 | h-UCN2 | 9.37²/9.12⁵ | 9.22 | 0.04 | 0.72 | 0.05 | 128.9 | 2.95 | 40 |
| CRHR2 | h-SRP | ~9⁴ | 9.58 | 0.05 | 1.06 | 0.13 | 108.7 | 2.48 | 25 |
| CRHR2 | h-SRP | ~9⁴ | 9.23 | 0.03 | 0.99 | 0.06 | 98.56 | 1.42 | 36 |

Data in italic represents potency approximations; NA = data not available due to low potency and limited peptide supply; values from published data were obtained with the author's in-house synthesized peptides used for cAMP stimulation of the following transfected systems:
¹h-CRHR1 or
²m-CRHR2b transfected CHO-K1 cells (Lewis, K. et al., 2001, PAMS, vol. 98, pp. 7570-5);
³h-CRHR1 or
⁴h-CRHR2b transfected HEK-293 cells, approximated values from concentration response curves (Hsu, S. Y. et al., 2001, Nat. Med., vol. 7, pp. 605-11);
⁵m-CRHR2b transfected HEK-293 cells (Brauns, O. et al., 2002, Peptides, vol. 23, pp. 881-888).

The effects of amidation of the C terminal domain of h-SCP on agonist activity, in terms of potency and/or intrinsic activity, were investigated, since recombinant non-amidated peptide libraries would be difficult to assay in the CRHR2 transfected SK—N-MC cells.

To investigate the peptide agonist activity contribution of different amino acids, several modified peptides were synthesized, starting with 1-7 deletions within the N-terminal sequence. Each peptide was dissolved in water at stock concentrations of 1 mM and stored in Eppendorf tubes (Catalog No. 022364111) in aliquots at −40° Celsius. Peptides were thawed out only once, on the day of the experiment, and diluted further in the cAMP assay buffer.

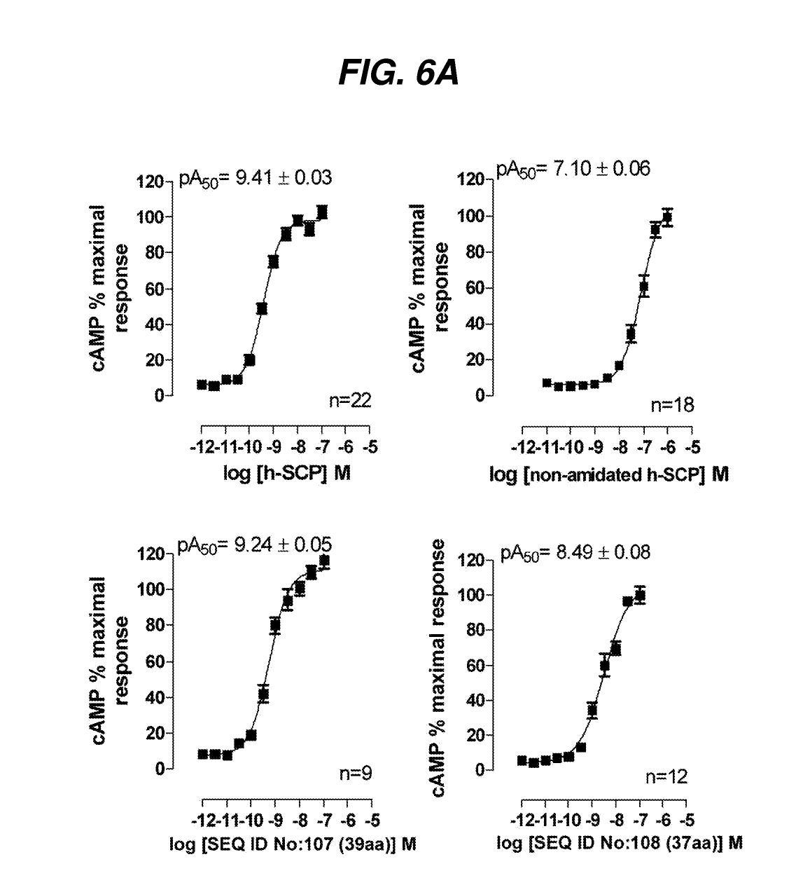

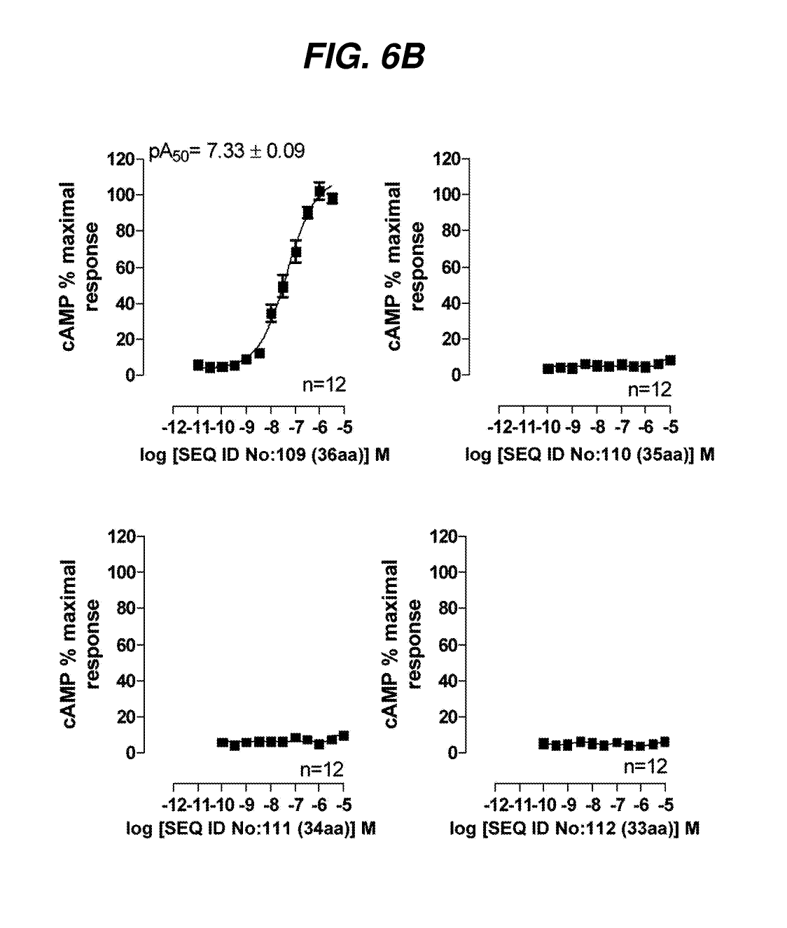

All peptides that produced cAMP in h-CRHR2 transfected SK—N-MC cells, achieved similar maximum responses within each experimental replicate. However the maximal response to h-SCP (SEQ ID NO:1) did vary between daily replicates, so the data were normalized to the maximum response to h-SCP obtained within each replication. Data were then combined from 3-5 replicate experiments for final calculation of the agonist concentration-effect curve parameters (FIG. 6). The pA₅₀ values obtained are summarized in Table 12.

Non-amidated h-SCP (SEQ ID NO:113) was approximately 200-fold less potent than the amidated parent peptide although the maximum response was indistinguishable. In one batch the parent 40 amino acid h-SCP peptide (SEQ ID NO:1) produced a pA₅₀ value of 9.41±0.03. Terminal amidation while important for potency is not essential and a fully defined concentration-effect curve was obtained with the non-amidated peptide with the same maximum response as the amidated parent peptide.

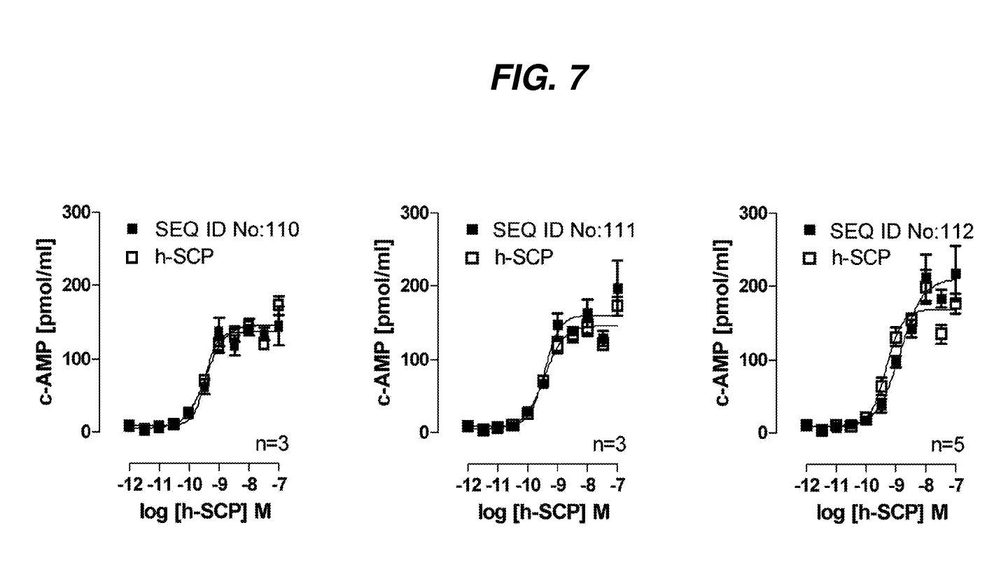

One amino acid deletion (SEQ ID NO:107) had no significant effect in potency (pA₅₀ 9.24±0.05), while the deletion of three (SEQ ID NO:108) and four (SEQ ID NO:109) amino acids resulted in a progressive reduction in pA₅₀ values (8.49±0.08 and 7.33±0.9), respectively, and also listed in Table 12. The deletion of five or more amino acids (SEQ ID NO:110, SEQ ID NO:111 and SEQ ID NO:112) resulted in complete loss of agonist activity (FIG. 6). Accordingly, the latter three peptides were tested as antagonists of h-SCP at a concentration of 10 μM (FIG. 7). None of the peptides had a significant effect on the h-SCP concentration-effect curve indicating that the peptides not only had no detectable intrinsic efficacy, but also no significant receptor occupancy, i.e. affinity less than 10 μM.

N-terminal domain deletions of 4 or more amino acids on h-SCP sequence affect the peptide potency. Peptides with one to four amino-acid deletions of the N-terminal domain had progressive reduction in potency, while peptides with deletions of five or more amino-acids resulted in complete loss of agonist activity and receptor affinity (K_{A} >10 μM). The later was expected, based on a previous report of a similar analysis performed on h-UCN2 (Isfort, R. J. et al., 2006, Peptides, vol. 27, pp. 1806-1813), since the deletions are close to the conserved amino-acid serine in position 6 and the aspartic acid in position 8.

TABLE 12

| SEQ ID | | | | | | | |
|---|---|---|---|---|---|---|---|
| No. | pA₅₀ | SEM | n_{H} | SEM. | αₘₐₓ | SEM | n |
| 1 | 9.41 | 0.04 | 1.18 | 0.11 | 98.68 | 1.59 | 22 |
| 113 | 7.10 | 0.06 | 1.07 | 0.13 | 107.4 | 5.45 | 18 |
| 107 | 9.25 | 0.05 | 1.07 | 0.12 | 111.3 | 2.52 | 9 |
| 108 | 8.49 | 0.08 | 0.82 | 0.10 | 106.3 | 5.05 | 12 |
| 109 | 7.34 | 0.09 | 0.74 | 0.10 | 109.6 | 6.16 | 12 |
| 110 | NR | | | | | | 12 |
| 111 | NR | | | | | | 12 |
| 112 | NR | | | | | | 12 |

NR = no response

Furthermore, the effects of cysteine mutation, N-ethylmaleimide capping, and pegylation on the peptide agonist activity was investigated. Control pA₅₀ of h-SCP (SEQ ID NO:1) varied for the various assay batches from 9.47 to 9.74 with SEM of 0.03 to 0.11. Again, several modified peptides were synthesized according to the above Schemes, and the assay results for these peptides are listed in Table 13.

TABLE 13

| SEQ | | | Log | | SEQ | | | Log | |
|---|---|---|---|---|---|---|---|---|---|
| ID | | | DR | | ID | | | DR | |
| No. | pA₅₀ | SEM | [M] | SEM | No. | pA₅₀ | SEM | [M] | SEM |
| 2 | 8.97 | 0.02 | 0.72 | 0.03 | 55 | ~7.93 | | ~1.61 | |
| 3 | 8.97 | 0.03 | 0.72 | 0.03 | 56 | ~7.20 | | ~2.34 | |
| 4 | 8.65 | 0.06 | 1.03 | 0.07 | 57 | ~7.64 | | ~1.90 | |
| 5 | 8.93 | 0.04 | 0.76 | 0.05 | 58 | ~7.14 | | ~2.40 | |
| 6 | 9.07 | 0.04 | 0.61 | 0.05 | 59 | ~7.22 | | ~2.32 | |
| 7 | 7.60 | 0.09 | 2.08 | 0.10 | 60 | ~6.32 | | >3.22 | |
| 8 | ~6.82 | | 2.86 | | 61 | ~6.22 | | >3.32 | |
| 9 | 7.80 | 0.06 | 1.89 | 0.07 | 62 | ~6.06 | | >3.48 | |
| 10 | 8.28 | 0.08 | 1.30 | 0.09 | 63 | ~7.45 | | ~2.12 | |
| 11 | 8.76 | 0.06 | 0.82 | 0.07 | 64 | ~6.98 | | ~2.59 | |
| 12 | 7.86 | 0.10 | 1.72 | 0.11 | 65 | ~6.82 | | ~2.75 | |
| 13 | 9.59 | 0.04 | −0.01 | 0.06 | 66 | 8.31 | 0.04 | 1.26 | 0.05 |
| 14 | ~7.34 | | >2 | | 67 | ~6.35 | | >3 | |
| 15 | 8.68 | 0.04 | 0.90 | 0.06 | 68 | ~6.96 | | ~2.61 | |
| 16 | 8.93 | 0.03 | 0.76 | 0.03 | 69 | 7.45 | 0.05 | 2.09 | 0.05 |
| 17 | 9.50 | 0.07 | 0.02 | 1.02 | 70 | ~7.34 | | ~2.07 | |
| 18 | 8.41 | 0.09 | 1.11 | 1.72 | 71 | ~7.35 | | ~2.26 | |
| 19 | 8.01 | 0.04 | 1.67 | 0.04 | 72 | 8.04 | 0.04 | 1.50 | 0.04 |
| 20 | 9.00 | 0.08 | 0.52 | 0.74 | 73 | 8.29 | 0.10 | 1.11 | 0.18 |
| 21 | 8.75 | 0.06 | 0.77 | 1.44 | 74 | ~7.33 | | ~2.28 | |
| 22 | 9.17 | 0.04 | 0.52 | 0.04 | 75 | 8.24 | 0.06 | 1.30 | 0.06 |
| 23 | 8.55 | 0.03 | 1.13 | 0.04 | 76 | 6.84 | 0.09 | 2.70 | 0.09 |
| 24 | 8.94 | 0.03 | 0.74 | 0.03 | 77 | 8.27 | 0.05 | 1.27 | 0.05 |
| 25 | 9.17 | 0.08 | 0.35 | 2.51 | 78 | ~7.89 | | ~1.52 | |
| 26 | 9.44 | 0.04 | 0.08 | 2.58 | 79 | 8.50 | 0.12 | 1.11 | 0.15 |
| 27 | 8.76 | 0.10 | 0.76 | 2.61 | 80 | 7.60 | 0.10 | 1.75 | 0.15 |
| 28 | 9.36 | 0.07 | 1.61 | 0.09 | 81 | 7.83 | 0.03 | 1.82 | 0.07 |
| 29 | 9.47 | 0.06 | 0.00 | 0.07 | 82 | 8.40 | 0.15 | 1.12 | 0.19 |
| 30 | 8.40 | 0.05 | 1.28 | 0.05 | 83 | 7.91 | 0.05 | 1.63 | 0.05 |
| 31 | 8.02 | 0.08 | 1.61 | 0.09 | 84 | ~6.82 | | ~2.84 | |
| 32 | 9.41 | 0.05 | 0.11 | 2.80 | 85 | 8.51 | 0.08 | 0.89 | 0.17 |
| 33 | 9.07 | 0.06 | 0.45 | 2.83 | 86 | 8.79 | 0.12 | 0.82 | 0.15 |
| 34 | ~6.32 | | >3.19 | | 87 | ~6.00 | | >3.68 | |
| 35 | 8.93 | 0.06 | 0.70 | 0.07 | 88 | 8.12 | 0.03 | 1.55 | 0.04 |
| 36 | 9.10 | 0.07 | 0.42 | 2.88 | 89 | 8.48 | 0.08 | 0.98 | 0.14 |
| 37 | 8.58 | 0.10 | 1.05 | 0.11 | 90 | ~7.49 | | ~2.17 | |
| 38 | ~6.67 | | >2.95 | | 91 | ~6.23 | | >3.43 | |
| 39 | 9.21 | 0.04 | 0.41 | 0.06 | 92 | 8.12 | 0.03 | 1.55 | 0.04 |
| 40 | 9.08 | 0.04 | 0.55 | 0.06 | 93 | 8.20 | 0.04 | 1.47 | 0.04 |
| 41 | 7.45 | 0.27 | 2.07 | 2.94 | 94 | ~7.00 | | >2.39 | |
| 95 | 9.31 | 0.12 | 0.43 | 0.14 | 42 | ~7.75 | | ~1.72 | |
| 96 | 8.74 | 0.11 | 1 | 0.13 | 43 | 9.79 | 0.04 | −0.32 | 0.05 |
| 97 | ~9.00 | | ~0.74 | | 44 | ~7.5 | | ~1.97 | |
| 98 | 9.50 | 0.10 | 0.18 | 0.13 | 45 | 9.48 | 0.05 | −0.01 | 0.06 |
| 99 | 8.94 | 0.1 | 0.8 | 0.12 | 46 | 9.43 | 0.05 | 0.04 | 0.06 |
| 100 | 8.64 | 0.07 | 1.1 | 0.1 | 47 | 9.5 | 0.06 | −0.03 | 0.07 |
| 101 | 7.84 | 0.13 | 1.9 | 0.15 | 48 | 9.44 | 0.05 | 0.03 | 0.06 |
| | | | | | 49 | 9.36 | 0.06 | 0.11 | 0.07 |
| | | | | | 50 | 9.48 | 0.06 | −0.01 | 0.07 |
| | | | | | 51 | 8.79 | 0.04 | 0.68 | 0.05 |
| | | | | | 52 | 9.42 | 0.04 | 0.05 | 0.05 |
| | | | | | 53 | ~7.25 | | ~2.22 | |
| | | | | | 54 | 9.55 | 0.04 | −0.08 | 0.05 |

Results exemplifying the activity profile of various modifications of the inventive polypeptide are shown in the Table 14 including stresscopin (h-SCP) polypeptide, urocortin 2 (h-UCN2), and h-SCP-IA-PEG polypeptide (SEQ ID NO:102), with h-SCP-IA-PEG being a peptide having the SCP sequence with a cysteine substitution in position 28 as set forth in SEQ ID NO:29 and a PEG polymer linked via an acetamide (IA) linker to the cysteine in position 28. The data are the mean±SEM of one to three replicates and are expressed as the % of the maximum response obtained to h-SCP within each replicate experiment.

TABLE 14

| SEQ ID | | | | | | | |
|---|---|---|---|---|---|---|---|
| No. | pA₅₀ | SEM | n_{H} | SEM | αₘₐₓ | SEM | n |
| 1 | 9.40 | 0.02 | 1.26 | 0.08 | 100.1 | 1.11 | 28 |
| 115 | 9.51 | 0.02 | 1.34 | 0.09 | 116.9 | 1.33 | 24 |
| 102 | 8.15 | 0.02 | 1.05 | 0.05 | 111.1 | 1.95 | 32 |

The h-SCP-IA-PEG polypeptide was also incubated in the presence of 100 nM anti-sauvagine-30 a selective competitive antagonist of h-CRHR2 receptor, resulting in a rightward shift in the h-SCP-IA-PEG polypeptide concentration-response curve with corresponding pA₅₀ approximate value of 6.89, when maximal response was constrained to 100%.

### Study No. 2: CRHR1 and CRHR2 Radioligand Binding Activity

The binding profile of h-SCP (SEQ ID NO:1) at CRHR2 was determined in radioligand binding studies in a membrane preparation of SK—N-MC cells stably transfected with human CRHR2 using [¹²⁵I]-anti-sauvagine-30 as the radiolabel. The cells were harvested by cell scraping and resulting pellets immediately frozen at −80° Celsius (approximately 50×10⁶ cells/pellet).

Frozen cell pellets were defrosted on ice in 15 ml of assay buffer that was composed of 10 mM HEPES, 130 mM NaCl, 4.7 mM KCl, 5 mM MgCl₂, and 0.089 mM bacitracin at pH 7.2 and 21±3° Celsius. The solution was then homogenized with a Polytron tissue grinder at a setting of 10 and 7×3 s (Brinkmann Instruments, Westbury, N.Y.). The homogenate was centrifuged at 4° Celsius at 800×g for 5 min with the pellet being discarded. The supernatant was re-centrifuged at 26,892×g for 25 min at 4° Celsius with the final pellet being re-suspended in assay buffer. All binding assays were conducted in 96-well Multiscreen GF/B filter plates (Millipore, Billericay, Mass., U.S.A.) that were pre-soaked in assay buffer with 0.3% PEI for 1 hour. For competition studies, cell membranes of 45 μl volume were incubated with either 60 pM [¹²⁵I]-anti-sauvagine-30 in 50 μl volume for the CRHR2 assay or with)[¹²⁵I]-(Tyr⁰-sauvagine for the CRHR1 assay in the presence of 15 μl of competing ligand for 120 min having a total volume of 150 μl. Nonspecific binding was determined by inclusion of 1 μM of r-UCN1 (SEQ ID NO:114).

The bound radioactivity was separated by filtration using a Multiscreen Resist manifold (Millipore Corp., Billerica, Mass., U.S.A). The filters were washed three times with ice-cold PBS at pH 7.5 and radioactivity retained on the filters was quantified by its liquid scintillation measured by a TopCount counter (Packard BioScience, Boston, Mass., U.S.A). All experiments were performed in triplicate.

Data from individual competition curves were expressed as the percentage of specific [¹²⁵I]-anti-sauvagine-30 or)[¹²⁵I]-(Tyr⁰-sauvagine binding (B) within each experiment. These data were then analyzed using a four-parameter logistic using GraphPad Prism with the upper (αₘₐₓ) and lower (αₘᵢₙ) asymptotes weighted to 100% and 0%, respectively, by including these values two log units above and below the lowest and highest concentrations of the competitor, respectively:

$$B = \frac{{α}_{min} + ( {α}_{max} - {α}_{min} )}{1 + {10}^{( ( log {IC}_{50} - [ L ] ) {n}_{H} )}}$$

The competition curve obtained with h-SCP (SEQ ID NO:1) was biphasic. This indicated a high and low affinity receptor binding state characterized by a high negative logarithm of the concentration at 50% inhibition (pIC₅₀) and a low pIC₅₀ of 6.6. The high-affinity site binding was shown to be inhibited by 100 μM guanosine 5′-O-[gamma-thio]triphosphate (GTPγS). In contrast, h-UCN2 (SEQ ID No. 115) exhibited only high affinity binding suggesting that h-UCN2 behaved as an agonist with higher intrinsic efficacy than h-SCP (SEQ ID NO:1) in the assay. pK_{I} values resulting from this data analysis are shown in Table 15.

TABLE 15

| | Receptor | | | |
|---|---|---|---|---|
| SEQ Id | CRHR1 | | CRHR2 | |
| No. | pK_{I} | n_{H} | pK_{I} | n_{H} |
| 1 | 4.6 ± 0.28 | 1.16 ± 0.65 | 5.71 ± 0.04 | 1.00 ± 0.04 |
| 114 | 8.69 ± 0.15 | 0.91 ± 0.27 | 8.51 ± 0.05 | 1.19 ± 0.14 |
| 115 | ND | | 7.74 ± 0.05 | 1.28 ± 0.15 |
| 116 | 4.96 ± 1.69 | 0.79 ± 1.21 | 6.49 ± 0.07 | 0.68 ± 0.08 |
| 117 | ND | | 7.57 ± 0.04 | 1.26 ± 0.14 |
| 118 | 5.81 ± 0.20 | 1.00 ± 0.49 | 7.78 ± 0.05 | 1.15 ± 0.12 |

ND = Not detectable

### Study No. 3 Vascular Smooth Muscle Relaxation—Rat Aortic Rings

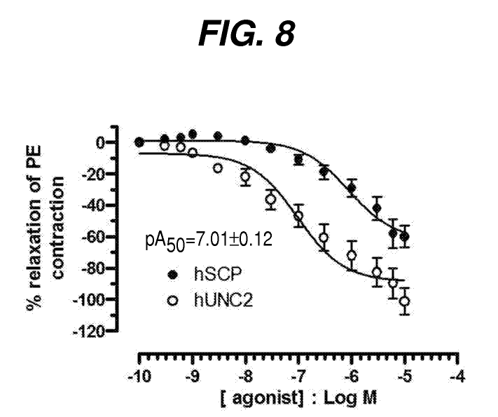

The ability of h-SCP (SEQ ID NO:1) to relax vascular smooth muscle was examined in isolated, rat aortic rings pre-contracted with phenylephrine (PE) (FIG. 8). This polypeptide (SEQ ID NO:1) produced concentration-dependent relaxation with a pA₅₀ of 6.05±0.12, but was 10-fold less potent than h-UCN2 (SEQ ID NO:115) having a pA₅₀ of 7.01±0.13. The responses caused by h-SCP (SEQ ID NO:1) were inhibited by anti-sauvagine-30 (SEQ ID NO:118).

### Study No. 4: Cardiovascular Characterization in Isolated Rabbit Heart

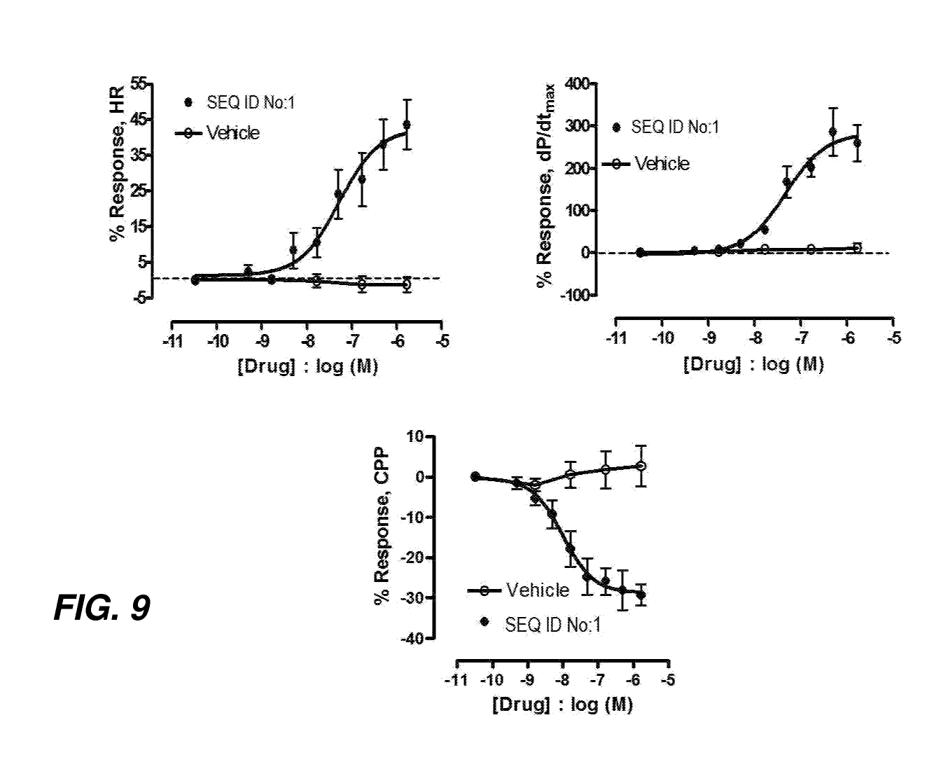

The effect of h-SCP (SEQ ID NO:1) on heart rate (HR), left ventricular (LV) contraction, and vascular tone was assessed in a retrograde-perfused Langendorff rabbit heart assay. A bolus of a placebo-like control vehicle or h-SCP (SEQ ID NO:1) was administered directly into the perfusion block. h-SCP (SEQ ID NO:1) produced concentration-dependent increases in heart rate and left ventricular developed pressure (dP/dtₘₐₓ) and a corresponding decrease in coronary perfusion pressure (CPP) at a concentration for 50% response equal to 52 nM, 9.9 nM, and 46 nM, respectively (FIG. 9), while no response was observed in case of the control vehicle.

### Study No. 5: Hemodynamics in Anaesthetized Rats (IV Bolus)

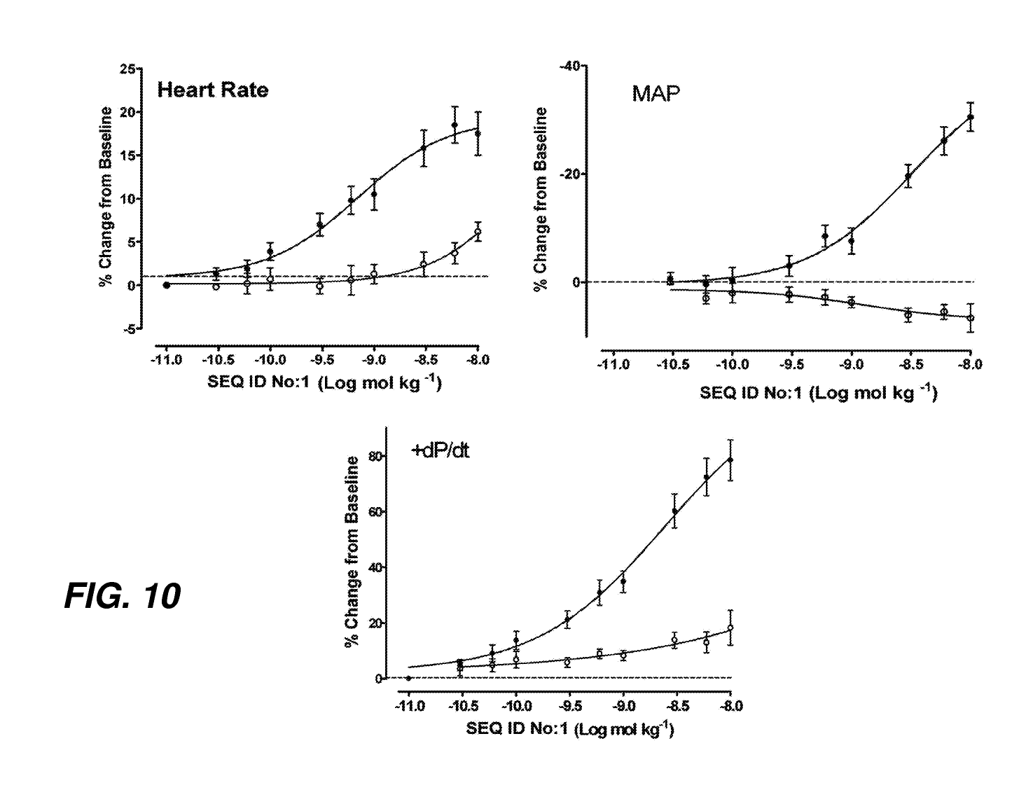

The hemodynamic profile of h-SCP (SEQ ID NO:1) was determined in sodium pentobarbital anaesthetized male Sprague-Dawley rats (FIG. 10). A SPR-320 Mikro-Tip® integrated catheter-tipped micro-manometer (Millar Instruments, Houston, Tex., U.S.A.) was placed in the right femoral artery for blood pressure measurements, and another one directly in the left ventricle for LV pressure measurement. Intravenous bolus administration of h-SCP (SEQ ID NO:1) over a dose range of 0.13 μg/kg to 44 μg/kg, equivalent to a range of 0.03 nmol/kg to 10 nmol/kg, produced dose-dependent increases in heart rate, LV developed pressure (+dP/dt), and a corresponding decrease in blood pressure, i.e. mean artery pressure (MAP). The changes in hemodynamic parameters induced by h-SCP (SEQ ID NO:1, full circle in FIG. 10) were blocked by pretreatment with anti-sauvagine-30 (SEQ ID NO:118, open circle in FIG. 10). Moreover, in these healthy anaesthetized rats anti-sauvagine-30 did not inhibit baseline parameters consistent with studies in conscious rats reported by Gardiner (Gardiner et al., J. Pharmacol. Exp. Ther., 2007, vol. 321, pp. 221-226).

### Study No. 6: Hemodynamics, Angiographic, and Echocardiographic Profile in Anaesthetized Healthy Dogs

The effects of h-SCP (SEQ ID NO: 1) on cardiovascular function were also assessed in anaesthetized mongrel dogs following intravenous bolus and 30-minute infusions. Hemodynamic and left ventricular systolic and diastolic function was evaluated using conventional hemodynamic, angiographic, echocardiographic, and radiographic methods with the results summarized in Table 16. Control vehicle or h-SCP (SEQ ID NO:1) was administered by intravenous bolus over a dose range of 0.13 μg/kg to 13.1 μg/kg, equivalent to a range of 0.03 nmol/kg to 3.0 nmol/kg. h-SCP (SEQ ID NO:1) produced dose-dependent changes in blood pressure, left ventricular systolic and diastolic function, and heart rate with the increase in heart rate of 45% being the largest in magnitude.

TABLE 16

| | Bolus Dose | | | | | | | | | | | |
|---|---|---|---|---|---|---|---|---|---|---|---|---|
| | (μg/kg) | | | | | | | | | | | |
| | VEH | | 0.13 | | 1.3 | | 2.6 | | 4.4 | | 13.1 | |
| | (nmol/kg) | | | | | | | | | | | |
| | | | 0.03 | | 0.3 | | 0.6 | | 1.0 | | 3.0 | |
| LVEDV | 56 | (1.8) | 55 | (1.9) | 54 | (1.8) | 52 | (2.1)* | 51 | (2.3)* | 46 | (2.0)* |
| LVESV | 26 | (0.8) | 25 | (1.1) | 23 | (1.2)* | 22 | (1.0)* | 22 | (1.0)* | 18 | (0.8)* |
| LVEDA | 13.0 | (0.4) | 12.9 | (0.4) | 12.7 | (0.3)* | 12.5 | (0.4)* | 12.4 | (0.3)* | 11.4 | (0.3)* |
| LVESA | 7.1 | (0.2) | 6.9 | (0.2) | 6.5 | (0.4)* | 6.2 | (0.3)* | 6.0 | (0.2)* | 5.2 | (0.2)* |
| LVFAS | 45 | (1.2) | 46 | (0.8) | 49 | (1.1)* | 50 | (1.6)* | 52 | (1.4)* | 53 | (1.5)* |
| LVEF | 53 | (1.2) | 55 | (0.8)* | 57 | (0.6)* | 57 | (0.5)* | 58 | (0.7)* | 60 | (0.7)* |
| SV | 29 | (1.3) | 30 | (1.0) | 30 | (1.1) | 30 | (0.9) | 30 | (1.4) | 28 | (1.3) |
| LV dP/dt | 1459 | (106) | 1546 | (177) | 1606 | (106) | 1675 | (111) | 1682 | (137) | 1760 | (128)* |
| PSAP | 94 | (2.1) | 92 | (4.2) | 91 | (3.1) | 90 | (2.8)* | 90 | (3.0)* | 88 | (2.0)* |
| HR | 74 | (3) | 73 | (5) | 85 | (6)* | 92 | (6)* | 94 | (6)* | 107 | (7)* |
| CO | 2.19 | (0.18) | 2.19 | (0.16) | 2.56 | (0.14)* | 2.72 | (0.17)* | 2.80 | (0.12)* | 2.86 | (0.12)* |

| | | | | | | |
|---|---|---|---|---|---|---|
| N | 7 | 7 | 7 | 7 | 7 | 7 |

LVEDV = LV end diastolic volume (mL)
LVEDA = LV end diastolic area (cm²)
LVFAS = LV fractional area of shortening (%)
SV = Stroke volume (mL)
PSAP = Peak systolic aortic pressure (mmHg)
N = number of dogs studied
*p < 0.05 vs vehicle (saline) control: Paired t-test
VEH = control vehicle (saline) = baseline
SEM = standard area of the mean
LVESV = LV end systolic volume (mL)
LVESA = LV end systolic area (cm²)
LVEF = LV ejection fraction (%)
LV + dP/dt = LV contractility (mmHg/sec),
HR = Heart rate (beats/min),
CO = Cardiac output (L/min)
Values = Mean (±s.e.m.) of the changes from VEH

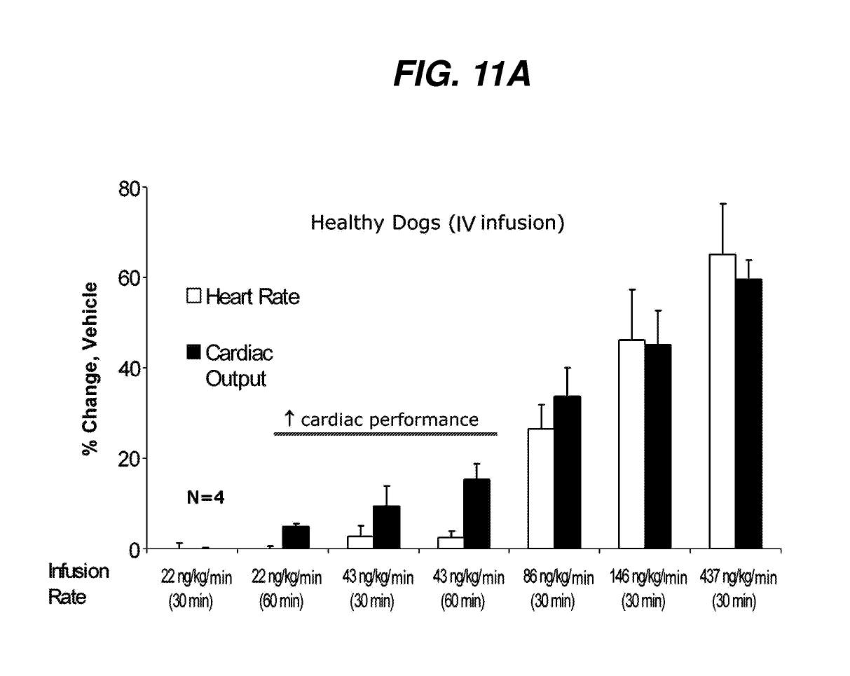

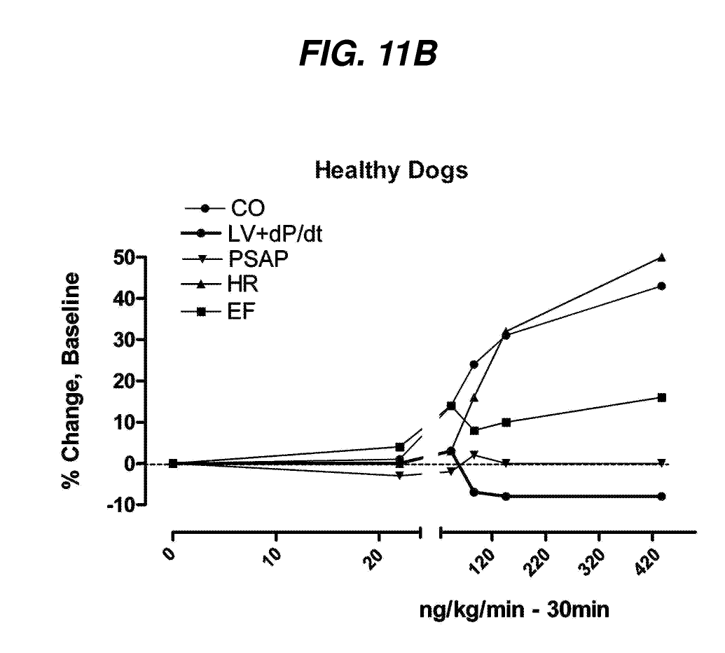

The findings described above were further examined in a study, in which h-SCP (SEQ ID NO:1) was infused over a 30-minute period at the same total doses that were administered by bolus as described above with the results presented in Table 17 and FIGS. 11A & B. As in the case of bolus dosing h-SCP (SEQ ID NO:1) elicited dose- (infusion-) dependent changes in blood pressure, left ventricular systolic and diastolic function, and heart rate. However, at the lower range of the dose- (infusion-) response curve there was a pronounced lessening in the positive chronotropic and blood pressure response with marked and significant increase in cardiac function measured as increased CO and LVEF. The infusion rate for minimal effect was 43 ng/kg/min, equivalent to 1.29 μg/kg total dose administered over 30 minutes. The corresponding plasma concentration of h-SCP (SEQ ID NO:1) was 4,577 pg/mL.

### Determination of Plasma Concentration

A sandwich immunoassay was developed using an affinity purified goat polyclonal antibody, specific to h-SCP that was pre-coated onto a microplate with integrated electrodes. h-SCP molecules present in the sample will bind to the capture polyclonal antibody coated on the plate. After washing away any unbound substances, a sulfo-tagged mouse monoclonal anti-h-SCP antibody was added. This conjugated antibody will bind to the h-SCP molecules captured on the microplate and the quantity of analyte was determined by electrochemiluminescence. The amount of signal generated is directly proportional to the h-SCP concentration in the sample or standard. The standard curve range is 3.125-1600 pg/mL with a quantifiable range from 10-800 pg/mL. A sample volume of 25 μL (in duplicate) is required for this assay. This immunoassay is specific for human and dog stresscopin and human urocortin III (h-UCN3). The assay does not recognize human stresscopin related peptide (h-SRP), urocortin I (h-UCN1) or urocortin II (h-UCN2). After completion of the analysis, based on a comparison of reference standards between the ELISA and HPLC method, a correction factor of 1.57 was applied to all bioanalytical data.

TABLE 17

| | IV rate (ng/kg/min) | | | | | | | | | | | |
|---|---|---|---|---|---|---|---|---|---|---|---|---|
| | VEH | | 22 | | 43 | | 86 | | 146 | | 437 | |
| | IV time (min) | | | | | | | | | | | |
| | 30 | | 30 | | 30 | | 30 | | 30 | | 30 | |
| LVEDV | 55.7 | (1.1) | 53.0 | (0.8) | 55.2 | (1.9) | 54.2 | (2.2) | 49.5 | (2.9)* | 46.0 | (3.7)** |
| LVESV | 27.2 | (1.1) | 24.5 | (0.5) | 23.5 | (1.9) | 23.5 | (1.2) | 20.7 | (1.2)** | 18.2 | (1.4)** |
| LVEDA | 12.2 | (0.15) | 12.2 | (0.3) | 11.9 | (0.2) | 11.8 | (0.4) | 11.5 | (0.3)* | 10.8 | (0.5)** |
| LVESA | 6.5 | (0.2) | 6.3 | (0.2) | 5.8 | (0.4) | 5.9 | (0.4) | 5.6 | (0.4)* | 5.1 | (0.5)** |
| LVFAS | 46.8 | (1.1) | 49 | (0.4) | 51.5 | (3.3) | 50.0 | (2.0) | 51 | (1.8) | 53.5 | (2.2)* |
| LVEF | 50.8 | (1.6) | 54.0 | (0.6) | 57.5 | (3.2) | 56.5 | (1.5) | 58.2 | (1.8)* | 60.7 | (1.9)** |
| SV | 28.7 | (0.7) | 28.5 | (0.5) | 31.7 | (1.9) | 30.7 | (1.5) | 28.7 | (2.2) | 28 | (2.8) |
| LV + dP/dt | 1623 | (55.8) | 1598 | (135) | 1875 | (167.8) | 1622 | (81.4) | 1594 | (93.4) | 1657 | (97.9) |
| PSAP | 97.2 | (2.5) | 95.0 | (6.2) | 95.2 | (5.1) | 91.7 | (2.4) | 90.0 | (0.8) | 89.5 | (0.9) |
| HR | 83.4 | (1.8) | 81.2 | (1.8) | 88.7 | (4.4) | 91.0 | (5.7) | 102.5 | (9.4)* | 117.3 | (9.5)**⁺ |
| CO | 2.39 | (0.08) | 2.32 | (0.09) | 2.82 | (0.20) | 2.81 | (0.27) | 2.97 | (0.37)* | 3.25 | (0.45)** |
| [SEQ ID NO: 1] | | | | | | | | | | | | |
| (pg/mL) | 70.3 | (19.0) | 620 | (81.4) | 4,577 | (1577) | 5141 | (878) | 23,614 | (2432) | 67,148 | (1298) |
| (pmol/L) | 16.1 | (4.4) | 142 | (18.6) | 1,048.1 | (361.1) | 1177.3 | (201.1) | 5407.6 | (556.9) | 15376.9 | (297.2) |
| Total Dose | | | | | | | | | | | | |

| | | | | | | |
|---|---|---|---|---|---|---|
| (ng/kg) | 0.0 | 0.66 | 1.29 | 2.58 | 4.38 | 13.11 |
| (nmol/kg) | 0.0 | 0.15 | 0.30 | 0.59 | 1.00 | 3.00 |
| N | 12 | 4 | 4 | 4 | 4 | 4 |

[X] = plasma concentration of compound X
⁺Junctional Tachycardia,
*p < 0.05 vs vehicle (saline) control,
**p < 0.005 vs vehicle (saline) control: Unpaired t-test

Study No. 7: Hemodynamics, Angiographic, and Echocardiographic Profile in Anaesthetized Dogs with Advanced Heart Failure (HF)

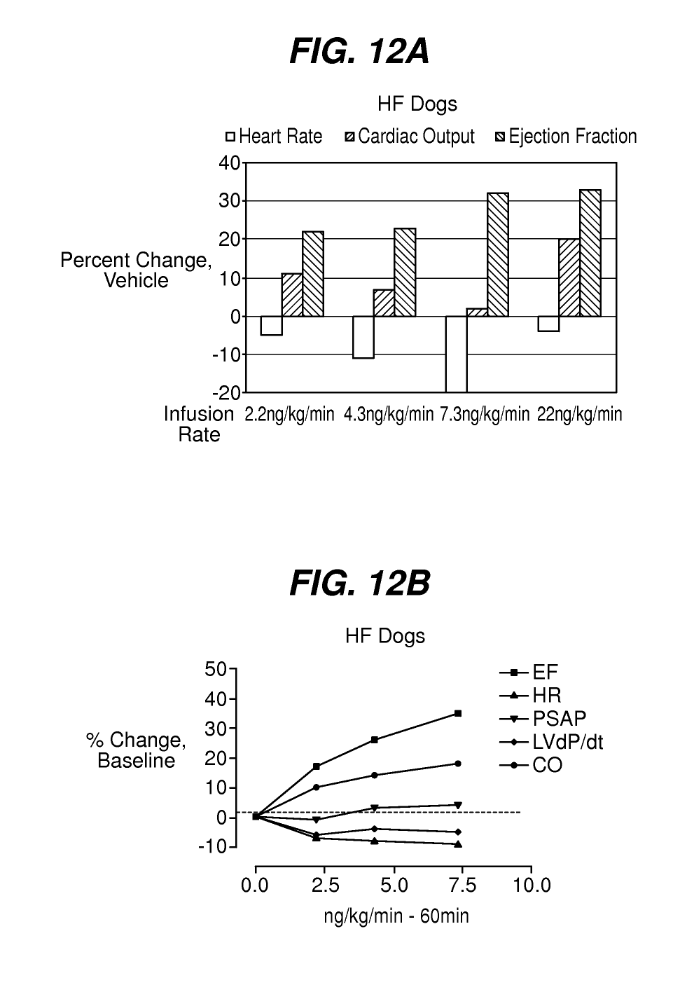

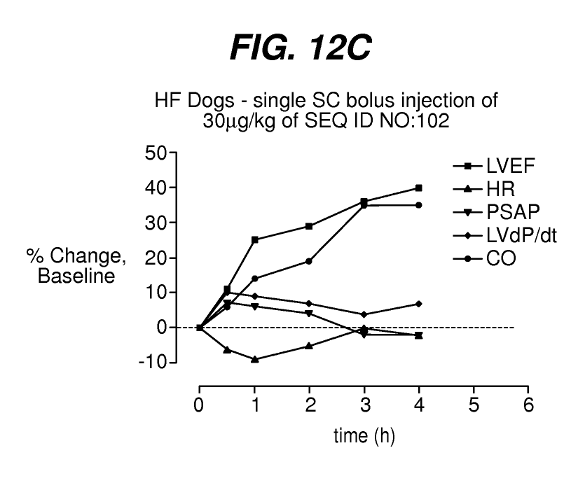

The effects of h-SCP (SEQ ID NO:1) on cardiovascular function were also assessed in anaesthetized dogs with advanced, irreversible heart failure of ischemic etiology (Sabbah et al., 1991, Am. J. Physiol., vol. 260, pp. H1379-H1384; Sabbah et al., 1994, Circulation, vol. 98, pp. 2852-2859; Chandler et al., 2002, Circ. Res., vol. 91, pp. 278-280). Progressive, advanced heart failure was produced in mongrel dogs by multiple sequential intracoronary microembolization with polystyrene latex microspheres. Dose infusions of 2.2, 4.3, and 7.3 ng/kg/min were administered intravenously over 60 minutes just following or just prior to hemodynamic, angiographic, echocardiographic, and Doppler measurements using conventional hemodynamic, angiographic, echocardiographic, and radiographic methods. The h-SCP polypeptide (SEQ ID NO:1) produced dose- (infusion-) dependent increases in LVEF and SV and decreases in left ventricular end diastolic pressure (LVEDP), left ventricular pressure during isovolumic relaxation (LV-dP/dt), systemic vascular resistance (SVR), and left ventricular end-systolic volume (LVESV) that correlated with plasma concentration. No significant change in heart rate, peak systolic aortic blood pressure, LV+dP/dt, mean pulmonary artery pressure, mean pulmonary artery wedge pressure, right atrial pressure, or myocardial oxygen consumption were recorded following any of the 1 hour intravenous infusions (Table 18 and FIGS. 12 A & B). The improvement in LV systolic and diastolic function was not associated with the development of de novo ventricular arrhythmias.

TABLE 18

| | IV rate (ng/kg/min) | | | |
|---|---|---|---|---|
| | VEH | 2.2 | 4.3 | 7.3 |
| | IV time (min) | | | |
| | 60 | 60 | 60 | 60 |
| HR (beats/min) | 80 ± 3 | 76 ± 2 | 73 ± 3 | 74 ± 2 |
| PSAP (mmHg) | 92 ± 2 | 90 ± 3 | 87 ± 2 | 87 ± 2 |
| LVEDP (mmHg) | 15 ± 0.6 | 13 ± 0.9⁺ | 13 ± 0.4⁺ | 12 ± 0.6⁺⁺ |
| LV+dP/dt (mmHg/sec) | 1614 ± 144 | 1477 ± 104 | 1400 ± 71 | 1398 ± 74 |
| LV−dP/dt (mmHg/sec) | 1350 ± 154 | 1216 ± 44 | 1094 ± 42⁺ | 1112 ± 82⁺ |
| MPAP (mmHg) | 16 ± 0.8 | 15 ± 0.5 | 15 ± 0.5 | 14 ± 0.4 |
| PAWP (mmHg) | 11 ± 0.6 | 9.0 ± 0.6 | 10 ± 0.6 | 9.0 ± 0.3 |
| RAP (mmHg) | 6.1 ± 0.5 | 5.7 ± 0.5 | 5.4 ± 0.4 | 5.0 ± 0.4 |
| SVR (dynes-sec-cm⁻⁵) | 4922 ± 143 | 4414 ± 193 | 4144 ± 243⁺ | 3958 ± 182⁺⁺ |
| EDV (mL) | 67 ± 2.5 | 66 ± 2.5 | 65 ± 2.5 | 64 ± 2.4 |
| -ESV (mL) | 49 ± 2.0 | 46 ± 1.7 | 43 ± 1.7 | 41 ± 1.8⁺ |
| EF (%) | 27 ± 0.5 | 31 ± 0.5^{+++,a,b} | 33 ± 0.5^{+++,c} | 35 ± 0.9⁺⁺⁺ |
| SV (mL) | 18 ± 0.6 | 20 ± 0.9⁺ | 22 ± 0.9⁺⁺ | 22 ± 0.9⁺⁺ |
| CO (L/min) | 1.41 ± 0.06 | 1.56 ± 0.11 | 1.61 ± 0.13 | 1.67 ± 0.09 |
| LVCBF (mL/min) | 46 ± 3.0 | 52 ± 5 | 57 ± 6 | 59 ± 6 |
| LV Efficiency (%) | 18.7 ± 2.0 | 23.0 ± 3.1 | 26.0 ± 4.4 | 23.3 ± 3.2 |
| MVO₂ (μmols/min) | 218 ± 22 | 191 ± 14 | 177 ± 19 | 196 ± 18 |
| [SEQ ID NO: 1] | | | | |
| (pg/mL) | 21.3 ± 8.0 | 141.4 ± 18.2 | 178.3 ± 21.1 | 279.1 ± 29.6 |
| (pmol/L) | 4.9 ± 3.0 | 32.4 ± 4.2 | 40.8 ± 4.8 | 63.9 ± 6.8 |
| Total Dose | | | | |
| (μg/kg) | 0.0 | 0.13 | 0.26 | 0.44 |
| (nmol/kg) | 0.0 | 0.03 | 0.06 | 0.10 |
| N | 7 | 7 | 7 | 7+ |

LVEDP = left ventricular end diastolic pressure
LV+dP/dt = left ventricular pressure during isovolumic contraction
LV−dP/dt = left ventricular pressure during isovolumic relaxation
MPAP = mean pulmonary artery pressure
PAWP = mean pulmonary artery wedge pressure
SVR = systemic vascular resistance
LVCBF = total left ventricular coronary blood flow
ACSO₂ dif = arterial coronary sinus oxygen difference
MVO₂ = myocardial oxygen consumption
RAP = mean right atrial pressure
⁺p < 0.05 vs baseline,
⁺⁺p < 0.01 vs baseline,
⁺⁺⁺p < 0.001 vs baseline,
^{a}p < 0.01 vs 4.3 ng/kg/min,
^{b}p < 0.001 vs 7.3 ng/kg/min,
^{c}p < 0.05 vs 7.3 ng/kg/min: Analysis of variance (ANOVA)

The results of this study indicate that an acute 60-minute intravenous administration of h-SCP (SEQ ID NO:1) dose-dependently improves LV (systolic and diastolic) function in dogs with advanced heart failure. The actions of h-SCP (SEQ ID NO:1) on cardiovascular function were rapid in onset and rapidly reversible. The improvement in LV function appears to result from changes in LV end systolic and diastolic dimension in that left ventricular end-diastolic volume (LVEDV) and LVESV decrease as left ventricular stroke volume (SV) increases. These changes occurred with no positive chronotropy (increase in heart rate), inotropy (increase in LV+dP/dt), or increase in MVO₂. The marked improvement in LV function was plasma concentration-dependent and not associated with any apparent increase in de novo ventricular arrhythmias.

In order to determine the threshold effective dose-infusion in dogs with advanced heart failure, a further study was performed at lower dose infusions. In addition, the opportunity was taken to explore whether the increase in LVEF produced by higher dose-infusions of 4.3 ng/kg/min would remain stable over a longer infusion period, i.e. 120 minutes. The results are presented in Table 19.

TABLE 19

| | IV rate (ng/kg/min) | | | | |
|---|---|---|---|---|---|
| | VEH | 0.22 | 0.43 | 4.3 | 4.3 |
| | | IV time (min) | | | |
| | | 60 | 60 | 60 | 120 |
| HR (beats/min) | 78 ± 1.6 | 75 ± 1.1 | 77 ± 1.0 | 79 ± 2.0 | 81 ± 3.6 |
| PSAP (mmHg) | 96 ± 4.8 | 97 ± 3.3 | 93 ± 3.6 | 92 ± 4.1 | 92 ± 4.3 |
| LVEDP (mmHg) | 14 ± 0.9 | 14 ± 1.1 | 13 ± 1.4 | 12 + 1.4 | 12 ± 1.2 |
| LV+dP/dt (mmHg/sec) | 1863 ± 96 | 1842 ± 127 | 1691 ± 96 | 1667 ± 88 | 1640 ± 88 |
| LV−dP/dt (mmHg/sec) | 1635 ± 171 | 1448 ± 155 | 1249 ± 120 | 1166 ± 82 | 1124 ± 92 |
| MPAP (mmHg) | 14 ± 0.8 | 15 ± 0.7 | 15 ± 0.8 | 15 ± 0.8 | 15 ± 0.9 |
| PAWP (mmHg) | 9.9 ± 0.5 | 10.1 ± 0.6 | 9.6 ± 0.7 | 9.0 + 0.6 | 9.4 ± 0.8 |
| SVR (dynes-sec-cm⁻⁵) | 4651 ± 341 | 4757 ± 287 | 4134 ± 195 | 3638 ± 191^{+,d} | 3372 ± 238^{++,a,c} |
| LVEDV (mL) | 67 ± 1.5 | 66 ± 1.5 | 65 ± 1.1 | 63 ± 1.3 | 62 ± 1.3 |
| LVESV (mL) | 49 ± 1.1 | 48 ± 1.2 | 45 ± 1.2 | 42 ± 1.4^{++,a} | 39 ± 1.4^{+++,b,e} |
| LVEF (%) | 27 ± 0.4 | 28 ± 0.6 | 30 ± 0.9 | 34 ± 1.4^{+++,b,e} | 37 ± 1.2^{+++,b,e} |
| SV (mL) | 18 ± 0.5 | 18 ± 0.5 | 19 ± 0.5 | 21 ± 0.8^{+++,a,c} | 23 ± 0.6^{+++,a,e} |
| CO (L/min) | 1.39 ± 0.05 | 1.37 ± 0.04 | 1.50 ± 0.04 | 1.68 ± 0.06^{++,a,c} | 1.83 ± 0.09^{+++,b,e} |
| [SEQ ID NO: 1] | | | | | |
| (pg/mL) | 32.7 ± 13.5 | 41.2 ± 14.9 | 37.2 ± 13.9 | 229 ± 41.6 | 249 ± 47.9 |
| (pmol/L) | 7.5 ± 3.1 | 9.4 ± 3.4 | 8.5 ± 3.2 | 52.4 ± 9.5 | 57 ± 11 |
| Total Dose | | | | | |
| (μg/kg) | 0.0 | 0.013 | 0.026 | 0.26 | 0.52 |
| (nmol/kg) | 0.0 | 0.003 | 0.006 | 0.06 | 0.12 |
| N | 7 | 7 | 7 | 7 | 7 |

⁺p < 0.05 vs baseline,
⁺⁺p < 0.01 vs baseline,
⁺⁺⁺p < 0.001 vs baseline,
^{a}p < 0.01 vs 0.22 ng/kg/min,
^{b}p < 0.001 vs 0.22 ng/kg/min,
^{c}p < 0.05 vs 0.43 ng/kg/min,
^{d}p < 0.05 vs 0.22 ng/kg/min,
^{e}p < 0.01 vs 0.43 ng/kg/min: ANOVA.

These data show that the infusion dose with minimal effect on hemodynamic, ventriculographic, and Doppler measurements of left ventricular systolic and diastolic function in dogs with advanced heart failure was 0.43 ng/kg/min that is equivalent to 25.8 ng/kg total dose administered over 60 minutes. The corresponding plasma concentration of h-SCP (SEQ ID NO:1) was 37.2 pg/mL. In addition the cardiovascular effects of a h-SCP (SEQ ID NO:1) dose-infusion of 4.3 ng/kg/min were stable between 60 and 120 minutes with no evidence of tachyphylaxis, including a diminished response.

In order to understand the potential cardiovascular effects of neutralizing antibody formation to h-SCP (SEQ ID NO:1), SV30 (SEQ ID No. 118), a competitive antagonist of CRHR2, was administered to dogs (N=4) with advanced heart failure. Our studies demonstrate that CRHR2 blocking doses of SV30 in dogs with advanced heart failure were without effect on cardiovascular parameters. This same infusion dose of SV30 blocked the actions of h-SCP (SEQ ID NO:1) in dogs with heart failure as shown in Table 20. These experiments with SV30 indicate that baseline cardiovascular parameters in dogs with advanced heart failure were not dependent upon endogenous hormone stimulation of CRHR2. Similar findings have been reported in healthy conscious and anaesthetized rats (Gardiner et al., J. Pharmacol. Exp. Ther., 2007, vol 321, pp. 221-226).

This suggests that the primary effect of neutralizing antibodies to h-SCP (SEQ ID NO:1) would not result in cardiac function that is further impaired from pre-treatment concentrations in healthy individuals or patients with heart failure.

TABLE 20

| | | | [SEQ ID NO: 1] = |
|---|---|---|---|
| | VEH | AS-30 | 4.3 ng/kg/min + AS-30 |
| HR (beats/min) | 77 ± 3 | 79 ± 3 | 82 ± 4 |
| PSAP (mmHg) | 98 ± 4 | 94 ± 5 | 90 ± 4 |
| LVEDP (mmHg) | 15 ± 1 | 15 ± 1 | 15 ± 2 |
| LV+dP/dt (mmHg/sec) | 1729 ± 171 | 1675 ± 109 | 1618 ± 79 |
| MPAP (mmHg) | 16 ± 0.5 | 16 ± 0.7 | 16 ± 0.8 |
| LVEDV (mL) | 69 ± 2.5 | 68 ± 2.7 | 68 ± 2.1 |
| LVESV (mL) | 50 ± 1.8 | 50 ± 1.9 | 49 ± 1.8 |
| LVEF (%) | 27 ± 0.4 | 27 ± 0 | 28 ± 0.5 |
| SV (mL) | 19 ± 0.6 | 18 ± 0.8 | 19 ± 0.4 |
| CO (L/min) | 1.43 ± 0.05 | 1.44 ± 0.04 | 1.55 ± 0.08 |
| N | 4 | 4 | 4 |

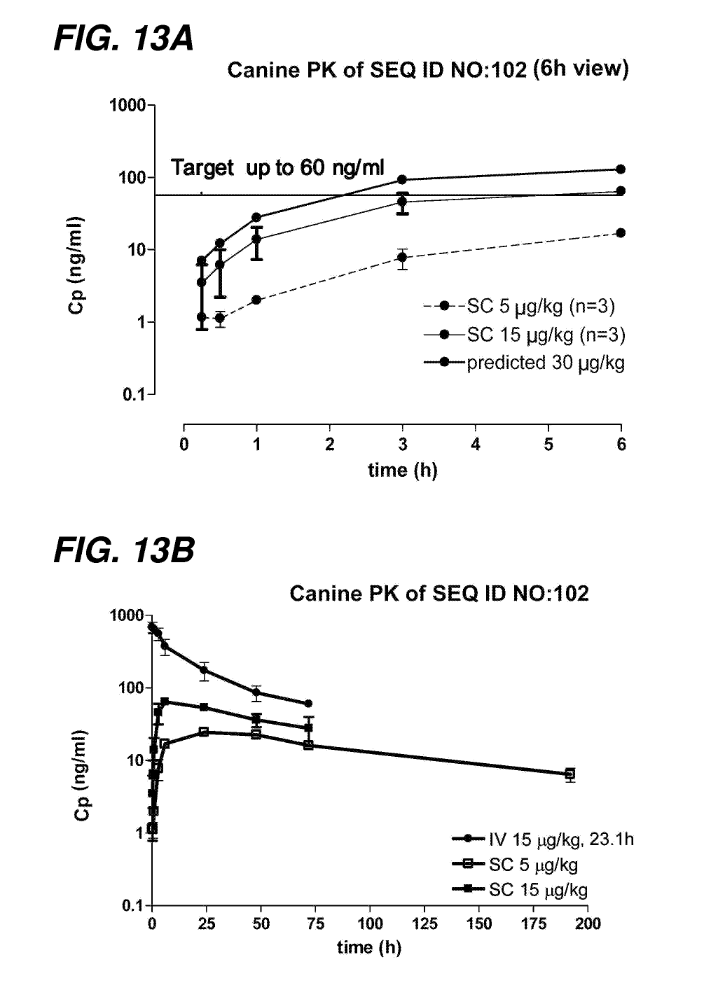

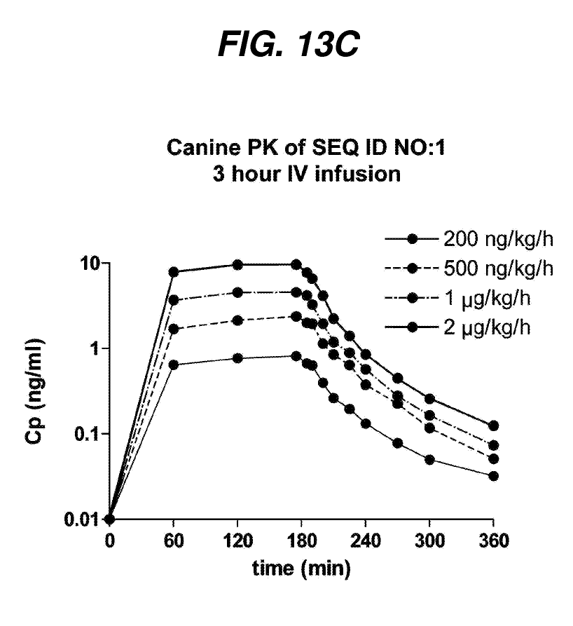

Results of a bolus SC injection of 30 μg/kg of a stresscopin-like peptide of SEQ ID NO:102 in HF dogs are shown in FIG. 12C. The heart rate declined over the first few hours, although the plasma concentration increased as predicted according to pharmacokinetic studies of bolus injection at lower doses (FIGS. 13A & B). After reaching a steady state plasma concentration, the heart rate remained fairly stable. Meanwhile, the LVEF and CO performance significantly increased over the same time period of up to 4 hours. The target plasma concentration of about 60 ng/mL is reached in about 2 hours and 10 minutes after the time point of injection, then leveling off at about 100 ng/mL after about 3, still maintaining its level at about 6 hours after injection. The stresscopin-relative concentration of 60 ng/mL and of 100 ng/mL of a SEQ ID NO:102 peptide is 600 pg/mL and 1000 pg/mL, respectively.

In summary, at lower dose infusions (≦7.3 ng/kg/min in dogs with heart failure), h-SCP increased LVEF, SV, and CO with no positive chronotropic, inotropic, or increases in myocardial oxygen consumption in dogs with ischemic induced, advanced, irreversible, and progressive heart failure. Furthermore, at these low doses the marked improvement in left ventricular function was not associated with decreases in PSAP, increases in heart rate, or any apparent increase in de novo ventricular arrhythmias and was readily reversible. In dogs with heart failure, the effective dose for significant increases in LVEF and CO was 0.43 ng/kg/min with a corresponding plasma concentration of 37.2 pg/mL.

In a subsequent study baseline hemodynamic, ventriculographic, echocardiographic and LV pressure-volume was measured, before each dog was intravenously administered a continuous, 4.3 ng/kg/min infusion of h-SCP (SEQ ID NO:1) for 120 min. At the end of the 120-min infusion, complete hemodynamic, ventriculographic, echocardiographic, and LV pressure-volume measurements were repeated. Lead II on the electrocardiogram was monitored throughout the study for development of de novo ventricular arrhythmias. The dosing solutions were not adjusted or corrected for peptide content since the peptide content of the test article used in these studies fell between the customary 85-90% limit where this correction is not required. Venous blood samples were obtained at baseline and after the hemodynamic evaluation following the 120-min h-SCP infusion.

All hemodynamic measurements were made during left and right heart catheterizations in anaesthelized dogs at each specified study time point. Aortic and LV pressures were measured using cathetenip micromanometers (Millar Instruments, Houston, Tex.), and LV end-diastolic pressure (LVEDP) was measured from the LV pressure waveform. Left ventriculography was performed during cardiac catheterization after completion of the hemodynamic measurements. Ventriculography were recorded on digital media at 30 frames per second during a power injection of 15 mL of contrast material (Conray; Mallinckrodt Inc., St. Louis, Mo.). Correction for image magnification was made using a radiopaque grid placed at the level of (he left ventricle. LV end-systolic volume (LVESV) and LV end-diastolic volume (LVEDV) were calculated from angiographic silhouettes using the area length method. Premature beats and postextrasystol beats were excluded from the analysis. LVEF was calculated as the ratio of the difference between LVEDV and LVESV to LVEDV times 100. Stroke volume (SV) was calculated as the difference between LVEDV and LVESV. Cardiae output (CO) was calculated as the product of heart fate and stroke volume. Systemic vascular resistance (SVR) was calculated as the quotient of mean arterial pressure and CO. The LV pressure-volume relationship was measured during a transient balloon occlusion of the inferior vena cava to assess the slope of the end-systolic pressure-volume relationship (ESPVR) and end-diastolic pressure-volume relationship (EDPVR). The end-systolic and end-diastolic pressure-volume points were determined for beats at end-expiration in the usual fashion. Linear regression analysis was used to determine the slope the ESPVR and EDPVR. An increase in the slope of the ESPVR infers improvement in LV contractile performance while a decrease in the slope of the EDPVR infers an improvement in LV relaxation.

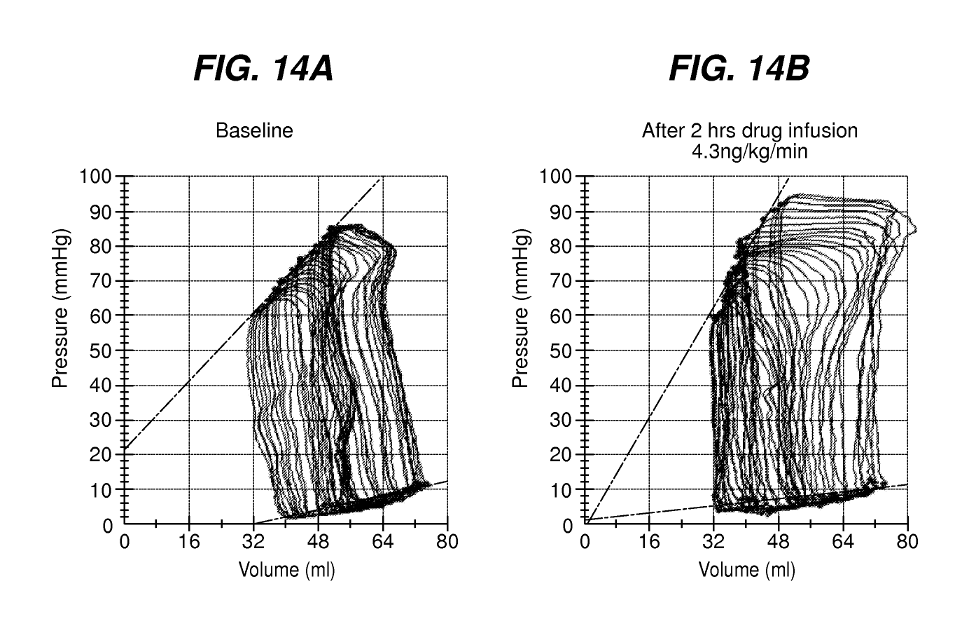

h-SCP (SEQ ID NO:1) produced marked, highly reproducible, plasma concentration dependent and statistically significant increases in global LV performance in dogs with advanced heart failure that manifested itself as increases in LVEF, SV, and CO with no change in MAoP, SAoP, HR, or LV+dP/dt. h-SCP (SEQ ID NO:1) also decreased LVESV to a far greater extent than it effects on decreasing LVEDV, thus likely altering the contractile state of the myocardium. FIG. 14A displays time-series data of LV pressure and volume measurements during transient inferior vena cava occlusion at baseline in dogs with heart failure. Two significant observations are made regarding these data. First, there was very little HR change during the few seconds required to obtain these measurements. Second, the inherent strength of the P-V loop technique to characterize cardiac specific alterations in intact animals. FIG. 14B illustrates the ESPVR as it shifts leftward and becomes steeper with infusion of h-SCP. The slope of the ESPVR in untreated dogs was 1.38±0.26 and increased to 2.26±0.46 in dogs with heart failure following h-SCP infusion. The absolute value of EDPVR slope was 0.257 in untreated dogs, while it was 0.128 in h-SCP treated dogs. This overall improvement in global LV systolic function was not associated with the development of de novo ventricular arrhythmias throughout the 120-min duration of this study.

h-SCP elicited changes in the geometry of the LV in general, and significant decreases in LVESV specifically; effects that translated into marked and significant increases in LVEF, LVSV, and CO without effecting LV+dP/dt, MAoP, SAoP, or HR. The key finding in the present study, specifically the marked and significant increase in the slope of the LV ESPVR following h-SCP infusion in dogs with advanced heart failure is a feature of the peptide that illustrates its load (preload and afterload) independent actions on the myocardium. Using real-time continuous LV pressure-volume analysis in the presence of vena cava occlusion, physiologic data consistent with the pharmacological profile of h-SCP resulting from effects that increased myocardial contractility to a greater extent and relaxation to a lesser extent were measured. Changes in the slope of the LV ESPVR contend the peptide acts on the myocardium, without excluding actions of vascular smooth muscle, in a manner that increases cardiac output by maintaining, and even increasing LVSV in the face of declining LV size without the development of de novo ventricular arrhythmias in these dogs.

### Study No. 8: Pharmacokinetics in Animals

The nonclinical pharmacokinetics of h-SCP (SEQ ID NO:1) and pegylated stresscopin-like peptides were studied in rats, dogs, and cynomolgus monkeys (cyno). The nonclinical pharmacokinetic studies and their results are presented in Table 21 and 22. Nonclinical pharmacokinetic studies focused on characterization of IV infusion at pharmacologically relevant dose levels, supplemented with IV and SC bolus and toxicokinetic analysis.

h-SCP (SEQ ID NO:1) plasma concentrations reached apparent steady-state within 1 hour after initiation of infusion in dogs (FIG. 13C) and cynomolgus monkeys, and within 2 hours in rats. In cynomolgus monkeys, h-SCP (SEQ ID NO:1) exhibited linear pharmacokinetics at dose levels of 16.7 to 100 ng/kg/min tested, with clearance values (CL) approximately 30 to 40 mL/min/kg. Compared to rats and cynomolgus monkeys, h-SCP (SEQ ID NO:1) had lower plasma clearance values in dogs at around 4 mL/min/kg, and exhibited linear pharmacokinetics over the pharmacologically relevant range from 3.3 to 33.3 ng/kg/min. However, plasma exposures of h-SCP (SEQ ID NO:1) in rats increased greater than dose-proportionally in both high-dose IV infusion of the toxicokinetic studies and bolus studies, with high clearance values from 42 to 116 mL/min/kg for IV bolus.

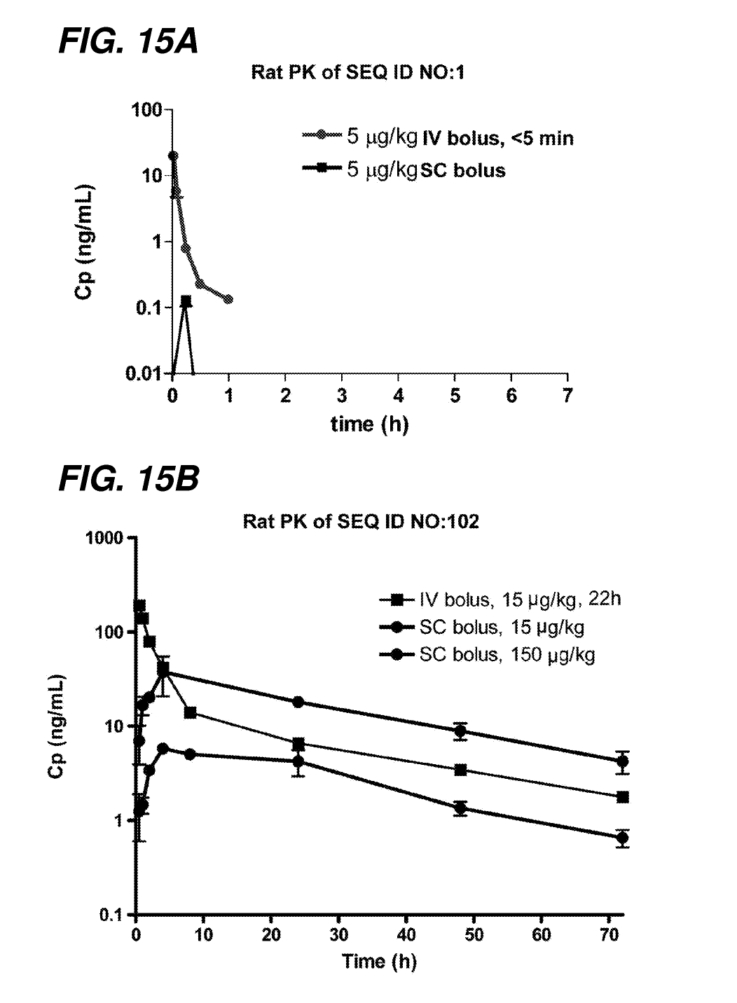

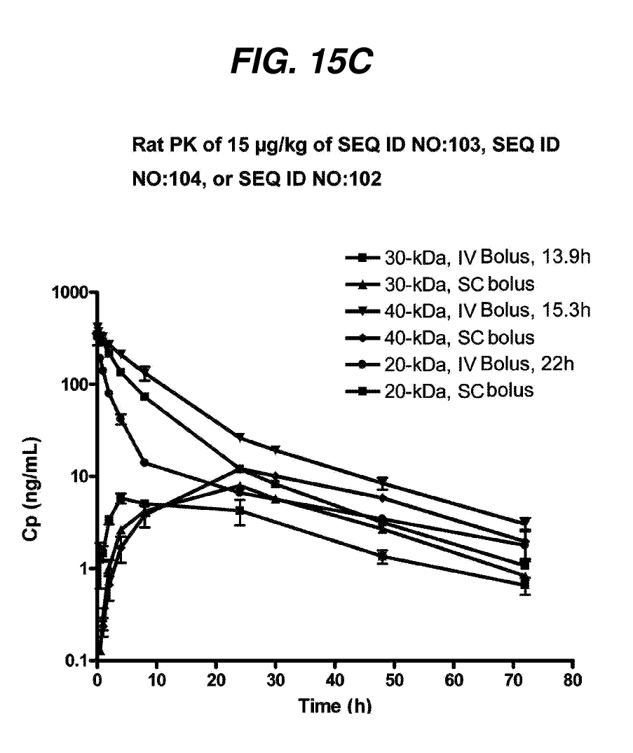

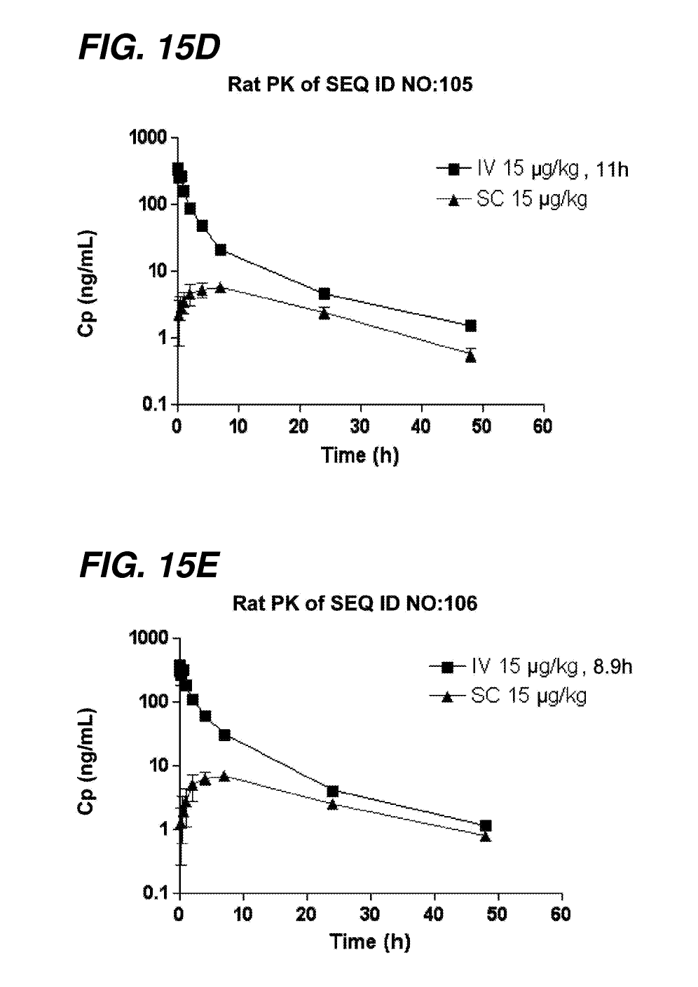

h-SCP (SEQ ID NO:1) showed a typical biphasic disposition profile following both IV infusion and bolus IV administrations, having a short initial phase of rapid concentration decline, and a longer terminal phase, i.e. in dogs of approximately 1 hour. Using two-compartment analysis, the alpha-phase half-life (t_{1/2 alpha}) was estimated to be less than 5 minutes in rats (FIG. 15A) and monkeys, and between 10 to 20 minutes in dogs. There was no evidence that the prolonged terminal half-life (t_{1/2 terminal}) had notable influence on the time needed to reach apparent steady state under continuous infusion. h-SCP reached steady-state concentrations within 1 hour in dogs and monkeys and within 2 hours in rats. The initial half-life of h-SCP is very short (<5 min in rats and monkeys and 10-20 min in dogs) followed by a longer terminal half-life (approximately 1 hour in dogs). There were no apparent gender differences in the pharmacokinetics of h-SCP in rats, dogs, or monkeys.

TABLE 21
Nonclinical Pharmacokinetic Studies of Peptide with SEQ ID NO: 1

| | | Dose | Cₘₐₓ | AUC_{0-∞} | CL | Vss | t_{1/2 terminal} | t_{1/2 alpha} |
|---|---|---|---|---|---|---|---|---|
| Study | Sex | (ng/kg/min) | (ng/mL) | (ng · min/mL) | (mL/min/kg) | (mL/kg) | (min) | (min) |
| Rat IV Infusion | M | 83.3 | 0.752 | 74.5 | 116.4 | 18377 | 113.4 | 3.0 |
| 3 hours | F | 83.3 | 0.906 | 88.1 | 106.6 | 19413 | 103.8 | 2.3 |
| 6M & 6F/group | M | 167 | 1.53 | 145.9 | 109.4 | 17643 | 110.8 | 1.3 |
| | F | 167 | 0.683 | 69.1 | 270.7 | 40895 | 59.4 | 3.2 |
| | M | 333 | 2.858 | 290.9 | 118.5 | 18597 | 63.5 | 2.1 |
| | F | 333 | 2.672 | 266.8 | 129.9 | 19067 | 58.2 | 1.9 |
| Rat IV Bolus | M | *3,000 | 4.8 | 31.3 | 109.3 | 565 | 6.7 | 1.8 |
| 3M/group | M | *10,000 | 13.6 | 89.8 | 115.8 | 1081 | 39.5 | 2.7 |
| | M | *50,000 | 224.5 | 1465.3 | 41.8 | 359 | 34.5 | 2.9 |
| | M | *300,000 | 780.2 | 5984.9 | 50.1 | 442 | 27.2 | 3.4 |
| Dog IV Infusion | M | 3.33 | 0.814 | 140.5 | 4.28 | 216 | 59.6 | 15.9 |
| 3 hours | M | 8.33 | 2.377 | 387.6 | 3.87 | 194 | 49.9 | 20.9 |
| 3M/group | M | 16.7 | 5.055 | 768.5 | 3.99 | 172 | 57.2 | 14.3 |
| | M | 33.3 | 9.996 | 1583.3 | 3.95 | 161 | 65.3 | 13.7 |
| Cyno IV Infusion | M | 16.7 | 0.873 | 103.7 | 30.2 | 788 | 7.4 | — |
| 3 hours | F | 16.7 | 0.613 | 78.6 | 39.7 | 848 | 5.3 | — |
| 2M & 2F/group | M | 33.3 | 1.481 | 208.1 | 29.2 | 611 | 26.0 | 3.7 |
| | F | 33.3 | 0.958 | 140.3 | 42.9 | 931 | 14.8 | 4.0 |
| | M | 100 | 4.447 | 587.7 | 30.7 | 899 | 94.3 | 2.9 |
| | F | 100 | 3.163 | 460.0 | 39.1 | 921 | 143.9 | 3.1 |

*ng/kg for the bolus injection data;
Vss = steady-state volume;
M = male,
F = female

Furthermore, the pharmacokinetics in rats and dogs of pegylated stresscopin-like peptides such as polypeptides of SEQ ID NO:102, 103, 104, 105, or 106 are shown in FIGS. 13A & 13B, and 15B to E, as well as in Table 22. The data continued to show a typical biphasic disposition profile following both IV infusion and bolus IV administrations, with the t_{1/2 alpha} values listed in Table 22.

TABLE 22
Pharmacokinetic Study of Peptide with SEQ ID NO: 102

| | Dose | Cₘₐₓ | AUC_{0-∞} | CL | Vz | t_{1/2 alpha} | tₘₐₓ | |
|---|---|---|---|---|---|---|---|---|
| Study | (μg/kg) | (ng/mL) | (ng · h/mL) | (mL/min/kg) | (mL/kg) | (h) | (h) | % F |
| Rat SC | 15 | 17.9 ± 8.4 | 342 ± 107 | | | | 7 ± 1 | 36 |
| Bolus | 150 | 77.6 ± 24.1 | 1914 ± 464 | | | | 6.3 ± 1.8 | 20 |
| Rat IV Bolus | 15 | | | 0.27 ± 0.02 | 510 ± 23 | 22 ± 0.5 | | |
| Dog SC | 5 | 24.6 ± 2.6 | 3510 ± 270 | | | | 32 ± 8 | 71 |
| Bolus | 15 | 66.8 ± 1.9 | 6089 ± 1808 | | | | 5 ± 1 | 41 |
| Dog IV Bolus | 15 | | | 0.02 ± 0.01 | 34 ± 7 | 21 ± 2 | | |

Vz = volume of distribution;
% F = Bioavailability

### Study No. 9: Human Dosing Studies

The minimal pharmacologically effective dose in dogs with heart failure was 0.43 ng/kg/min, which is notably lower than the minimally effective dose in healthy dogs (43 ng/kg/min). The NOAEL of 33.3 ng/kg/min was determined in a GLP cardiovascular safety study in male dogs, which is considered to be the most relevant and sensitive species for cardiovascular drugs.

Changes in heart rate seen in animals rapidly reverse following secession of infusion and are induced at a greater than 15-fold exposure margin below that where other effects are observed (body weight, reticulocyte decreases). Further, the non-cardiovascular effects seen in toxicology studies are relatively mild, monitorable, and reversible. h-SCP is relatively non-antigenic in animals, but in cases where antibody is induced, there appear to be no adverse physiologic consequences.

A NOAEL of 33.3 ng/kg/min was determined in a GLP cardiovascular safety study in male dogs, which is considered to be the most relevant and sensitive species for cardiovascular drugs. A nonclinical pharmacology study in healthy dogs showed the minimum anticipated biological effect level (MABEL) in dogs was 22 ng/kg/min (Table 17). Based on these values a starting dose of 0.1 ng/kg/min was selected.

Based on the pharmacokinetic-based approach, a starting dose of 0.1 ng/kg/min was expected to achieve a steady-state plasma concentration (Cpss) of 8.6 pg/mL, which is well below the upper limit of 12.0 ng/mL determined in a GLP cardiovascular safety study in dogs, and has a safety margin of 1,390-fold.

Furthermore, clinical studies indicated that the MABEL dose in healthy humans is similar to the MABEL dose in dogs determined in nonclinical pharmacology study, and that the human dose showing a cardiac response corresponded well with the dose in dogs.

Based on the below clinical studies the clearance (CL) in healthy humans of h-SCP (SEQ ID NO:1) following intravenous infusion was determined to be about 30 L/hr for a 70-kg man. At the infusion rate of 22 ng/kg/min in healthy dogs, the plasma concentration of h-SCP was determined to be 620 pg/mL (Table 17). A human equivalent dose of 4.4 ng/kg/min will be required to achieve a similar steady-state plasma concentration (Cpss) level of 620 pg/mL, as the dose can be calculated according to: doseₕᵤₘₐₙ=CLₕᵤₘₐₙ×Cpss/weightₕᵤₘₐₙ, with a human weighing 70 kg.

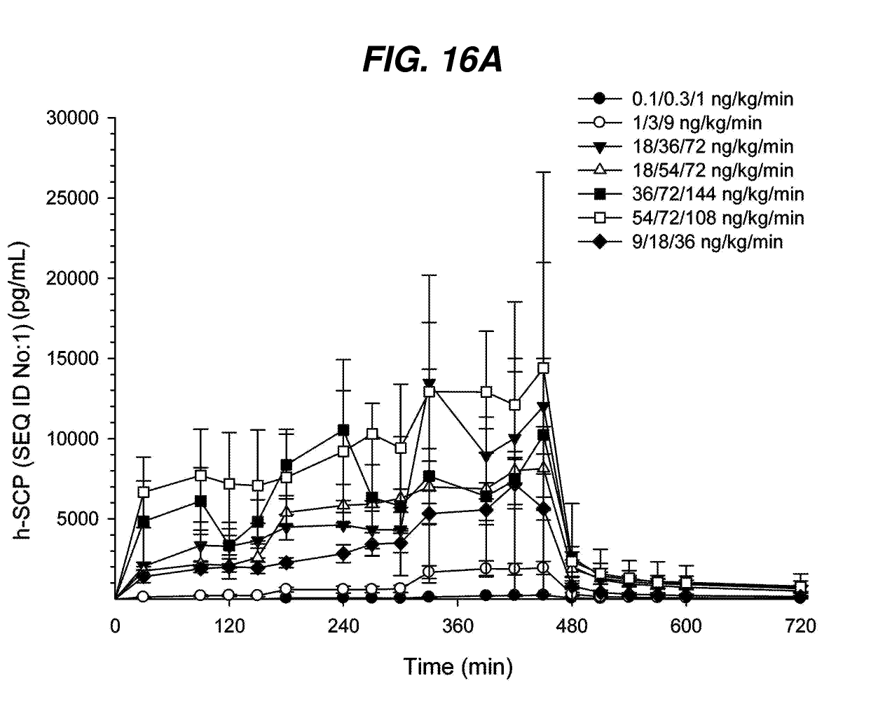

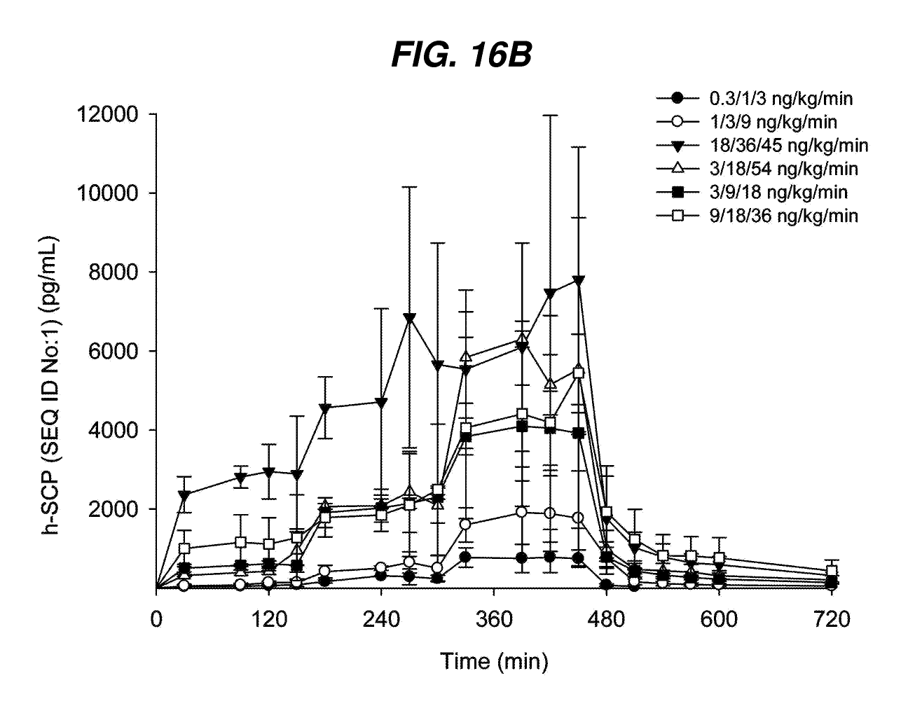

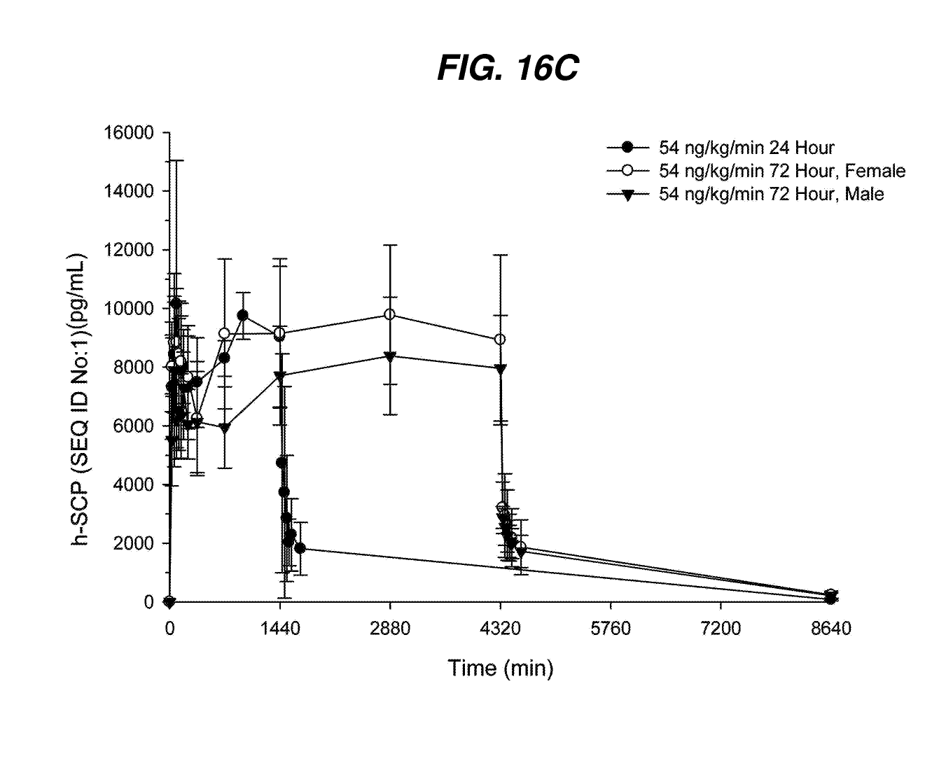

In healthy subjects following a 7.5-hour continuous ascending dose IV infusion of h-SCP (SEQ ID NO:1) noncompartmental pharmacokinetic analyses were performed to determine plasma concentrations of h-SCP (SEQ ID NO:1). Pharmacokinetic parameters of h-SCP (SEQ ID NO:1) are summarized in Table 23. Plasma h-SCP (SEQ ID NO:1) reached the steady state shortly after initiating the IV infusion (FIG. 16A). After the end of the infusion, plasma concentrations of h-SCP (SEQ ID NO:1) showed an initial rapid decline followed by a slower terminal elimination phase. Within 30 minutes, plasma h-SCP (SEQ ID NO:1) was reduced to ≦20% of the h-SCP (SEQ ID NO:1) level at the end of infusion. Mean terminal half-life ranged from 2.13 to 28.48 hours and appeared to increase with dose. The longer terminal half-lives at the higher doses suggested existence of a deeper compartment in addition to the normal 2-compartment model. However, the additional compartment's contribution to the overall exposure and accumulation of h-SCP (SEQ ID NO:1) is likely marginal as indicated by the effective half-lives. Mean effective half-life ranged from 1.54 to 14.17 hours. Mean systemic clearance was generally consistent across the dose groups and ranged from 0.27 to 0.42 L/kg.

TABLE 23
Mean (SD) Plasma Pharmacokinetic Parameters of h-SCP following a 7.5-Hour
Continuous Ascending Dose Intravenous Infusion in Healthy Subjects

| Infusion Rate | Tₘₐₓ | Cₘₐₓ | AUC_{inf} | T_{1/2} | T_{1/2}, | CL | Vₛₛ |
|---|---|---|---|---|---|---|---|
| (ng/kg/min) | (h)^{a} | (pg/mL) | (pg*h/mL) | (h) | effective (h) | (L/h/kg) | (L/kg) |
| 0.1/0.3/1 | 7.47 | 247.86 | — | — | 1.1. | 1.2. | 1.3. |
| (N = 5) | (6.50-7.50) | (55.02) | | | — | — | — |
| 1/3/9 | 7.00 | 2029.20 | 7405.62^{b} | 2.13^{b} | 1.54^{b} | 0.28^{b} | 0.61^{b} |
| (N = 5) | (6.50-7.42) | (458.71) | (1697.94) | (0.77) | (0.07) | (0.08) | (0.17) |
| 9/18/36 | 7.00 | 7259.60 | 28858.62 | 7.87 | 1.84 | 0.33 | 0.87 |
| (N = 5) | (5.50-7.42) | (1401.63) | (2253.19) | (0.67) | (0.37) | (0.03) | (0.19) |
| 36/72/144 | 7.42 | 29148.75 | 138065.13 | 28.48 | 14.17 | 0.27 | 5.60 |
| (N = 1) | | | | | | | |
| 18/36/72 | 6.46 | 14061.71 | 68718.30^{c} | 7.82^{c} | 2.82^{c} | 0.28^{c} | 1.12^{c} |
| (N = 2) | (5.50-7.42) | (5862.65) | | | | | |
| 18/54/72 | 5.50 | 9011.99 | 55862.26 | 16.21 | 6.89 | 0.41 | 3.46 |
| (N = 3) | (4.00-7.48) | (1737.44) | (15518.77) | (13.08) | (6.34) | (0.11) | (2.28) |
| 54/72/108 | 6.99 | 14638.48 | 95481.32 | 19.11 | 5.69 | 0.42 | 2.69 |
| (N = 2) | (6.50-7.47) | (6251.41) | (45226.59) | (15.12) | (5.06) | (0.19) | (1.41) |

^{a}Median (minimum-maximum);
^{b}N = 4;
^{c}N = 1.

In heart failure subjects following a 7.5-hour continuous ascending dose IV infusion of h-SCP (SEQ ID NO:1) noncompartmental pharmacokinetic analyses were performed on plasma concentrations of h-SCP (SEQ ID NO:1). Pharmacokinetic parameters of h-SCP (SEQ ID NO:1) are summarized in Table 24. The pharmacokinetics of h-SCP (SEQ ID NO:1) in heart failure subjects appeared to be similar to that of healthy subjects. Similar to what was seen in healthy subjects, plasma h-SCP (SEQ ID NO:1) reached steady state shortly after initiating the IV infusion in subjects with heart failure (FIG. 16B). After the end of infusion, plasma concentrations of h-SCP (SEQ ID NO:1) showed an initial rapid decline followed by a slower terminal elimination phase. Within 30 minutes, plasma h-SCP (SEQ ID NO:1) was reduced to equal or less than 20% of the h-SCP (SEQ ID NO:1) level at the end of the infusion (FIG. 16B). Mean systemic clearance ranged from 0.19 to 0.46 L/h/kg. Mean terminal half-life ranged from 0.24 to 7.04 hours, which is probably dose related as the highest infusion rate was only 54 ng/kg/min. The effective half-life ranged from 1.32 to 2.51 hours.

TABLE 24
Mean (SD) Plasma Pharmacokinetic Parameters of h-SCP following a 7.5-Hour Continuous
Ascending Dose Intravenous Infusion in Subjects with Heart Failure

| Infusion Rate | Tₘₐₓ | Cₘₐₓ | AUC_{inf} | T_{1/2} | T_{1/2}, | CL | Vₛₛ |
|---|---|---|---|---|---|---|---|
| (ng/kg/min) | (h)^{a} | (pg/mL) | (pg*h/mL) | (h) | effective (h) | (L/h/kg) | (L/kg) |
| 0.3/1/3 | 6.25 | 826.30 | 3402.04^{b} | 0.24^{b} | 1.32^{b} | 0.19^{b} | 0.36^{b} |
| (N = 2) | (5.50-7.00) | (319.20) | | | | | |
| 1/3/9 | 7.04 | 1981.13 | 6716.63 | 2.09 | 1.77 | 0.32 | 0.80 |
| (N = 2) | (6.53-7.55) | (897.29) | (2565.28) | (0.18) | (0.04) | (0.12) | (0.29) |
| 3/9/18 | 6.75 | 4770.98 | 18997.53 | 6.23 | 2.09 | 0.24 | 0.72 |
| (N = 2) | (6.50-7.00) | (84.72) | (118.13) | (0.38) | (0.07) | (0.00) | (0.03) |
| 9/18/36 | 7.48 | 5519.42 | 33820.67^{c} | 7.04^{c} | 3.95^{c} | 0.33^{c} | 1.74^{c} |
| (N = 3) | (6.50-7.58) | (3865.64) | (17155.95) | (3.56) | (0.82) | (0.16) | (0.54) |
| 18/36/45 | 7.34 | 8037.09 | 51517.76^{b} | 1.78^{b} | 1.12^{b} | 0.29^{b} | 0.47^{b} |
| (N = 2) | (7.05-7.63) | (3696.12) | | | | | |
| 3/18/54 | 6.04 | 6407.46 | 24858.81 | 7.04 | 2.51 | 0.46 | 1.64 |
| (N = 2) | (5.50-6.58) | (353.44) | (3803.66) | (0.05) | (0.35) | (0.07) | (0.02) |

^{a}Median (minimum-maximum);
^{b}N = 1;
^{c}N = 2.

In healthy subjects following a 24- or 72-hour infusion of 54 ng/kg/min of h-SCP (SEQ ID NO:1) noncompartmental pharmacokinetic analyses were performed on plasma concentrations of h-SCP (SEQ ID NO:1). Pharmacokinetic parameters of h-SCP (SEQ ID NO:1) are summarized in Table 25. The pharmacokinetics of h-SCP (SEQ ID NO:1) in healthy subjects following a 24- or 72-hour continuous IV infusion are similar to that with the 2.5-hour infusion with mean clearance ranging from 0.28 to 0.38 L/h/kg (FIG. 16C). Mean terminal half-life ranged from 23.40 to 28.81 hours and effective half-life ranged from 5.84 to 9.62 hours.

TABLE 25
Mean (SD) Plasma Pharmacokinetic Parameters of h-SCP Following a Continuous
Intravenous Infusion of 54 ng/kg/min in Healthy Subjects

| Infusion Rate | Tₘₐₓ | Cₘₐₓ | AUC_{inf} | T_{1/2} | T_{1/2}, | CL | Vₛₛ |
|---|---|---|---|---|---|---|---|
| (ng/kg/min) | (h)^{a} | (pg/mL) | (pg*h/mL) | (h) | effective (h) | (L/h/kg) | (L/kg) |
| 24 Hours Male | 16.00 | 12194.75 | 283260.75 | 25.68 | 9.37 | 0.28 | 3.70 |
| (N = 7) | (1.50-24.50) | (3616.17) | (45036.24) | (2.41) | (2.65) | (0.04) | (0.81) |
| 72 Hours Male | 24.00 | 10200.69 | 632354.60 | 28.81 | 9.62 | 0.38 | 4.94 |
| (N = 7) | (1.00-71.92) | (2318.31) | (97415.91) | (12.92) | (7.62) | (0.06) | (3.12) |
| 72 Hours Female | 18.01 | 11455.66 | 740379.08^{b} | 23.40^{b} | 5.84 | 0.33 | 2.72 |
| (N = 6) | (2.00-71.97) | (1608.09) | (181959.62) | (3.76) | (1.89) | (0.08) | (0.90) |

^{a}Median (minimum-maximum);
^{b}N = 5.

### Study No. 10: Human Efficacy Studies

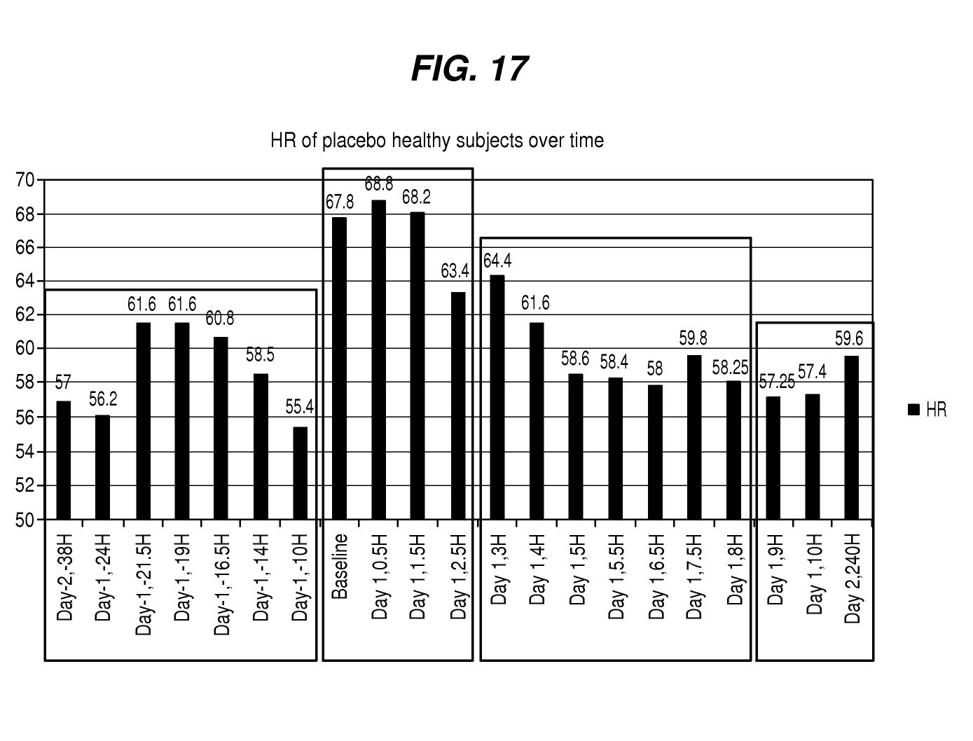

Efficacy was based on the pharmacodynamic evaluation of hemodynamics, which was monitored using the noninvasive technique of impedance cardiography. Heart rate values were collected by impedance cardiography measurements. It was noted that the heart rate of subjects receiving placebo were elevated on the day of their infusions, at baseline before the infusion, and for the first 3 to 4 hours after the infusions were started (FIG. 17). Based on this observation, it appeared that there was a potential effect of period on the observed heart rate.

A mixed-effect model with baseline as covariate, period and dose group (≦3 ng/kg/min−low, >3 to ≦36 ng/kg/min−mid, >36 ng/kg/min−high) as fixed effects, and a random subject effect was established using the heart rate change from baseline in healthy subjects. The model suggested both a statistically significant treatment effect (p<0.0001) and a statistically significant period effect (p=0.0171), but no statistically significant baseline effect (p=0.1931).

To confirm that the statistically significant increase in heart rate is caused by the high dose group, a similar mixed-effect model that excluded the high dose level (>36 ng/kg/min) group was built up. While this model still demonstrated a statistically significant period effect (p=0.0002), it did not show a statistically significant dose effect (p=0.1434) or a statistically significant baseline effect (p=0.3684).

### Post Hoc Graphical Analysis

A post hoc graphical analysis of the hemodynamic data was done to adjust for the elevated baseline values seen just before onset of the infusion, to obtain the best estimate of each hemodynamic parameter, and to correct for the effect of period. A post hoc graphic presentation was prepared from the complete (high frequency) dataset. This dataset contains the raw data that were further processed by the vendor (ie, CardioDynamics) and reported only at specific time points.

In this post hoc analysis an extended baseline was used for each value that included all values recorded before initiation of the infusion. Then an average value for each parameter was obtained from the last 30 minutes of each 2.5 hour infusion and was used as the effect in that period of the infused dose. Each value was modified for the effect of period of infusion by subtracting the mean change from baseline seen in placebo subjects dosed in that same period (placebo subtraction). The dose effect was estimated by averaging the values from all subjects who received the same dose after the placebo subtraction.

### Healthy Subjects, 7.5-Hour Continuous Ascending Dose IV Infusion

Subjects who received placebo had a mean decrease in heart rate from baseline heart rate (value obtained immediately before the infusion) of 5 to 10 bpm during the infusion. A review of the heart rate data in these subjects indicated that their heart rates were 5 to 10 bpm higher at baseline than their heart rates on the day before the infusion (FIG. 17). This suggests that subjects may have experienced anxiety before the start of the infusion that contributed to this increase in baseline heart rate values.

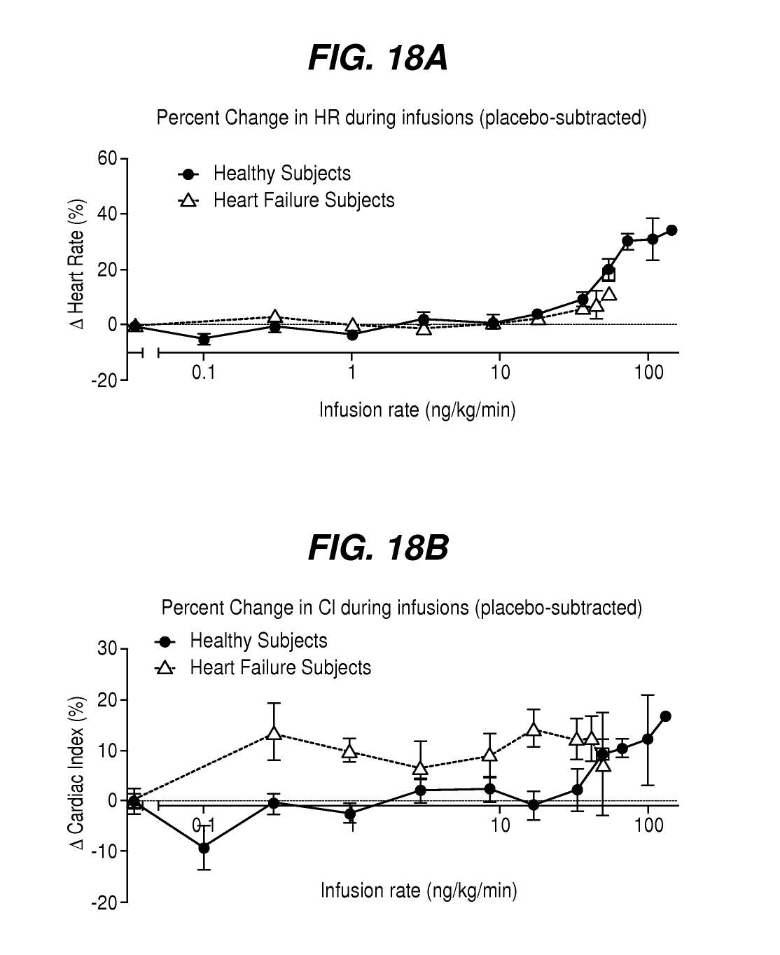

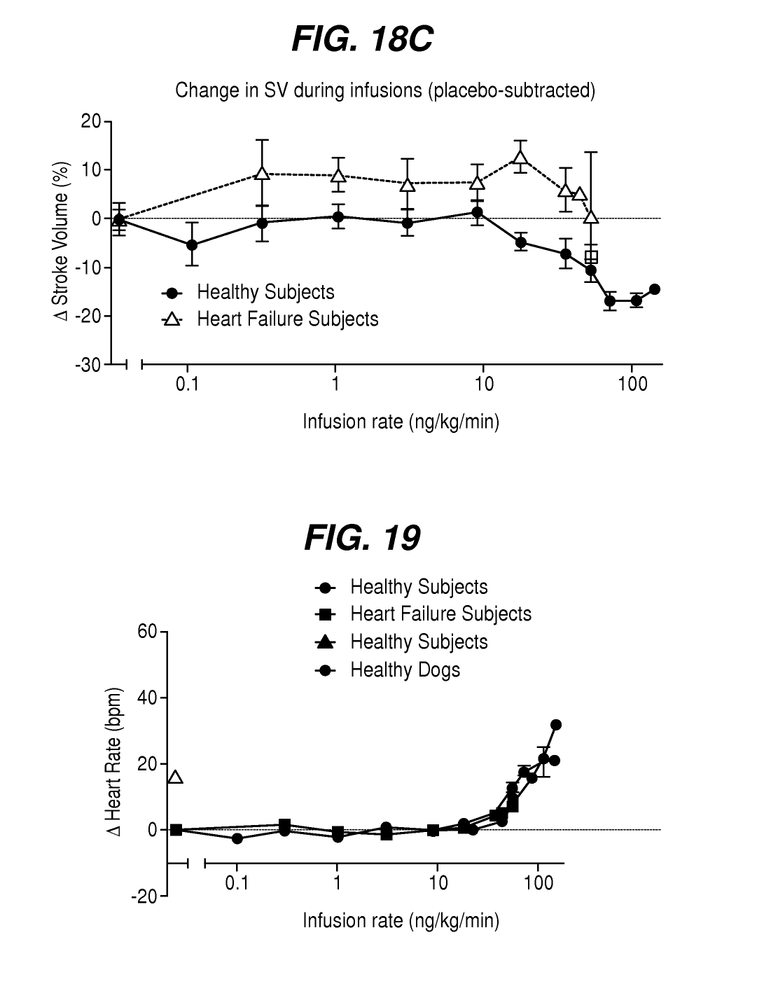

A similar decrease from baseline in heart rate was seen in healthy subjects receiving lower doses of h-SCP (SEQ ID NO:1). In contrast at the end of each 2.5-hour infusion period, subjects receiving doses of h-SCP (SEQ ID NO:1) ≧36 ng/kg/min had a dose-related increase in heart rate with an increase in heart rate from baseline that approached 30 bpm at doses of 72 ng/kg/min and higher (Table 26). The increase in heart rate was greater at higher doses of h-SCP (SEQ ID NO:1) (FIG. 18A). This increase in heart rate occurred at a dose similar to the h-SCP (SEQ ID NO:1) dose that resulted in an increased heart rate in dogs (FIG. 19).

Based on these observations it appears, that in healthy subjects, doses of h-SCP (SEQ ID NO:1) ≧36 ng/kg/min were associated with an increase in heart rate from baseline. This increase is particularly notable when compared with the decrease in heart rate seen in subjects receiving placebo. In contrast, in healthy subjects, doses of h-SCP (SEQ ID NO:1) less than 36 ng/kg/min had no notable increase in heart rate compared with baseline and the change from baseline was similar to that seen in subjects receiving placebo.

In healthy subjects, no change in cardiac output or cardiac index were seen at all doses of h-SCP (SEQ ID NO:1) ≦36 ng/kg/min. Subjects receiving doses greater than 36 ng/kg/min had an increase in cardiac output and cardiac index (FIG. 18B). These increases in cardiac output and cardiac index seen at these higher doses seem to be solely due to the increase in heart rate, since at these higher doses the stroke volume was decreased compared with baseline (FIG. 18C).

No clear trends in mean systolic and diastolic blood pressure were observed in placebo or at doses of h-SCP (SEQ ID NO:1) ≦108 ng/kg/min, but increases from baseline were observed at the highest dose (144 ng/kg/min) at the end of the infusion.

At the end of each 2.5-hour infusion period, mean systemic vascular resistance and mean systemic vascular resistance index were moderately increased from baseline in placebo and at doses less than 36 ng/kg/min, variable though generally unchanged at doses 36 through 72 ng/kg/min, and showed decreases from baseline at doses of h-SCP (SEQ ID NO:1) ≧108 ng/kg/min.

TABLE 26
Changes in Heart Rate in Healthy Subjects (Post Hoc Analysis)

| | Infusion Rate (ng/kg/min) | | | | | | | | | | | |
|---|---|---|---|---|---|---|---|---|---|---|---|---|
| | 0 | 0.1 | 0.3 | 1 | 3 | 9 | 18 | 36 | 54 | 72 | 108 | 144 |
| N | 5 | 5 | 5 | 10 | 5 | 10 | 10 | 7 | 6 | 8 | 2 | 1 |
| Baseline, bpm | 59.3 | 58.1 | 58.1 | 60.1 | 62.2 | 59.7 | 57.9 | 57.8 | 62.1 | 61.4 | 68.1 | 62.4 |
| | (4.2) | (1.5) | (1.5) | (1.5) | (2.4) | (1.5) | (2.8) | (1.1) | (5.2) | (3.9) | (2.4) | (0.0) |
| Change from | 0.0 | −2.8 | −0.4 | −2.0 | 1.3 | 0.1 | 2.1 | 5.2 | 12.7 | 18.1 | 20.9 | 21.2 |
| Baseline, bpm | (0.7) | (1.2) | (1.3) | (0.6) | (1.3) | (0.6) | (1.1) | (1.4) | (2.2) | (1.6) | (4.4) | (0.0) |
| Percent Change | 0.0 | −4.6 | −0.2 | −3.3 | 2.6 | 0.6 | 3.9 | 9.4 | 20.5 | 30.3 | 31.3 | 34.3 |
| from Baseline | (1.2) | (2.1) | (2.1) | (1.1) | (2.2) | (1.0) | (1.9) | (2.6) | (3.7) | (3.0) | (7.6) | (0.0) |

Overall, there was notable variability in the data for each hemodynamic parameter of the study. The high variability in hemodynamic parameters, confounded by the notable trend towards a decrease in mean heart rate during infusion (most evident in subjects receiving placebo), combined with the small number of subjects in each treatment group made it difficult to draw clear conclusions regarding results of the prespecified hemodynamic analysis. Post hoc analyses, designed to correct for these effects were performed to explore further the hemodynamic data.

Subjects with Stable Heart Failure, 7.5-Hour Continuous Ascending Dose IV Infusion

Subjects with heart failure who received placebo had a mean decrease in heart rate during the infusion from baseline heart rate (value obtained immediately before the infusion). A review of the heart rate data in these subjects indicated that their heart rates were higher at baseline than on the day before the infusion. As may have occurred in healthy subjects, subjects with stable heart failure may have experienced anxiety before the start of the infusion that contributed to this increase in baseline heart rate values.

A similar decrease in heart rate from baseline was seen in subjects with heart failure receiving doses of of h-SCP (SEQ ID NO:1) less than 36 ng/kg/min. In contrast, heart failure subjects receiving doses of h-SCP (SEQ ID NO:1) ≧36 ng/kg/min had an increase in heart rate compared with baseline (FIG. 18A). This increase in heart rate occurred at a dose similar to the h-SCP (SEQ ID NO:1) dose that resulted in an increased heart rate in healthy subjects and dogs (FIG. 19). Subjects receiving the highest dose of 54 ng/kg/min had an increase in heart rate that approached 10 bpm (Table 27).

TABLE 27
Changes in Heart Rate in Heart Failure Subjects (Post Hoc Analysis)

| | | Placebo | h-SCP (SEQ ID No: 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 63.1 | 67.2 | 65.7 | 65.1 | 66.1 | 64.7 | 64.8 | 59.7 | 64.4 |
| subjects | bpm | (4.2) | (6.2) | (2.7) | (3.3) | (4.1) | (3.6) | (4.5) | (6.3) | (5.4) |
| | CFB, | 0.0 | 1.5 | −0.4 | −1.3 | 0.2 | 0.8 | 3.6 | 3.8 | 7.0 |
| | bpm | (1.1) | (0.2) | (0.5) | (1.5) | (1.7) | (1.1) | (1.1) | (2.5) | (1.0) |
| | Percent | 0.0 | 3.0 | 0.2 | −0.9 | 1.5 | 2.5 | 6.3 | 7.5 | 11.5 |
| | CFB | (1.7) | (0.1) | (0.8) | (2.2) | (2.6) | (1.8) | (1.7) | (4.9) | (0.7) |

results: mean (standard error of the mean). The absolute and percent change from baseline values are placebo-subtracted.
N: number of subjects receiving each of the dose levels.
CFB = change from baseline.

Based on these observations it appears that in subjects with heart failure, doses of of h-SCP (SEQ ID NO:1) ≧36 ng/kg/min were associated with an increase in heart rate from baseline. This increase is particularly notable when compared with the decrease in heart rate seen in subjects receiving placebo. In contrast, in subjects with heart failure, doses of h-SCP (SEQ ID NO:1) less than 36 ng/kg/min had no clear increase in heart rate compared with baseline.

At the end of the 2.5-hour infusion period, mean cardiac output and cardiac index were decreased from baseline in placebo, while mean results were variable for all h-SCP (SEQ ID NO:1) doses. In contrast to healthy subjects, the response of cardiac output, cardiac index, and stroke volume to h-SCP (SEQ ID NO:1) in subjects with heart failure was detectable at all doses. Subjects with heart failure receiving h-SCP (SEQ ID NO:1) had an increase in cardiac index (and cardiac output) at all doses of h-SCP (SEQ ID NO:1) (FIG. 18B). This increase in cardiac index (and cardiac output) ranged from approximately 7% to 15%. No dose-response relationship was seen. The data indicates a potential effect of h-SCP (SEQ ID NO:1) on cardiac output, cardiac index, and stroke volume.

TABLE 28
Changes in Cardiac Output in Heart Failure Subjects (Post Hoc Analysis)

| | | Placebo | h-SCP (SEQ ID No 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 4.9 | 4.1 | 4.8 | 5.4 | 6.0 | 5.8 | 5.6 | 4.7 | 5.6 |
| subjects | L/min | (0.1) | (0.6) | (0.5) | (0.4) | (0.4) | (0.4) | (0.6) | (0.2) | (1.2) |
| | CFB, | 0.0 | 0.7 | 0.5 | 0.3 | 0.5 | 0.8 | 0.7 | 0.7 | 0.4 |
| | L/min | (0.1) | (0.3) | (0.1) | (0.3) | (0.3) | (0.2) | (0.3) | (0.2) | (0.8) |
| | Percent | 0.0 | 14.2 | 10.2 | 7.0 | 9.2 | 15.5 | 12.4 | 13.3 | 11.5 |
| | CFB | (2.5) | (6.0) | (2.4) | (4.9) | (4.3) | (4.1) | (4.2) | (4.6) | (14.1) |

Heart failure subjects receiving h-SCP (SEQ ID NO:1) at doses ≦36 ng/kg/min also had a clear increase in stroke volume (between 6% and 13%), seen at all of these lower doses (FIG. 19C). When doses greater than 36 ng/kg/min were infused the stroke volume was similar to baseline, suggesting at these higher doses that the increase in cardiac index was solely due to the increased heart rate.

TABLE 29
Changes in Cardiac Index in Heart Failure Subjects (Post Hoc Analysis)

| | | Placebo | h-SCP (SEQ ID No: 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 2.5 | 2.4 | 2.7 | 2.9 | 3.1 | 2.9 | 2.7 | 2.3 | 2.9 |
| subjects | L/min/m² | (0.1) | (0.6) | (0.3) | (0.2) | (0.1) | (0.2) | (0.2) | (0.2) | (0.1) |
| | CFB, | 0.0 | 0.3 | 0.2 | 0.1 | 0.2 | 0.4 | 0.3 | 0.3 | 0.2 |
| | L/min/m² | (0.1) | (0.1) | (0.1) | (0.1) | (0.1) | (0.1) | (0.1) | (0.1) | (0.3) |
| | Percent | 0.0 | 13.9 | 10.1 | 6.9 | 9.1 | 14.5 | 12.4 | 12.6 | 7.5 |
| | CFB | (2.5) | (5.7) | (2.3) | (4.9) | (4.4) | (3.8) | (4.1) | (4.4) | (10.1) |

TABLE 30
Changes in Stroke Volume in Heart Failure Subjects (Post Hoc Analysis)

| | | Placebo | h-SCP (SEQ ID No: 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 79.9 | 62.0 | 73.9 | 83.4 | 92.8 | 89.9 | 87.6 | 79.1 | 86.4 |
| subjects | mL | (5.4) | (4.3) | (7.4) | (6.0) | (6.6) | (5.7) | (9.1) | (4.5) | (11.1) |
| | CFB, | 0.0 | 7.6 | 6.6 | 4.8 | 5.9 | 9.3 | 4.2 | 3.3 | −2.5 |
| | mL | (2.4) | (4.3) | (2.9) | (4.6) | (3.6) | (2.9) | (3.5) | (0.2) | (12.2) |
| | Percent | 0.0 | 9.5 | 9.1 | 7.3 | 7.6 | 12.9 | 6.0 | 5.4 | 0.5 |
| | CFB | (3.3) | (6.6) | (3.4) | (5.1) | (3.7) | (3.3) | (4.5) | (0.2) | (13.3) |

Mean systolic and diastolic blood pressure were increased from baseline in placebo at the end of infusion. Conversely, mean systolic and diastolic blood pressure were decreased from baseline at the end of the infusion at all but one h-SCP (SEQ ID NO:1) dose (1 ng/kg/min), with larger decreases at doses of h-SCP (SEQ ID NO:1) ≧36 ng/kg/min. These blood pressure results were different from those seen in healthy subjects where there was no trend towards a decrease in blood pressure. In contrast to healthy subjects, subjects with heart failure receiving h-SCP (SEQ ID NO:1) had a decrease in systolic blood pressure and diastolic blood pressure at all doses of h-SCP (SEQ ID NO:1). This decrease in systolic blood pressure ranged from 5% to 21% and in diastolic blood pressure ranged from 9% to 24%. There was no evidence of an increased effect with higher doses in subjects receiving h-SCP (SEQ ID NO:1).

TABLE 31
Changes in Systolic Blood Pressure in Heart Failure Subjects (Post Hoc Analysis)

| | | Placebo | h-SCP (SEQ ID No: 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 107.6 | 116.5 | 123.1 | 116.1 | 120.2 | 112.5 | 115.4 | 104.7 | 110.8 |
| subjects | mm Hg | (4.7) | (11.2) | (6.2) | (6.3) | (6.7) | (6.6) | (9.2) | (2.4) | (25.4) |
| | CFB, | 0.0 | −18.2 | −4.9 | −9.5 | −10.6 | −8.8 | −16.4 | −12.0 | −18.8 |
| | mm Hg | (2.0) | (4.4) | (2.8) | (3.2) | (2.8) | (2.5) | (3.0) | (4.3) | (14.0) |
| | Percent | 0.0 | −15.8 | −4.7 | −9.2 | −9.4 | −8.7 | −14.9 | −11.7 | −21.4 |
| | CFB | (1.8) | (2.2) | (2.4) | (2.6) | (2.3) | (2.6) | (2.2) | (4.0) | (15.9) |

TABLE 32
Changes in Diastolic Blood Pressure in Heart Failure Subjects (Post Hoc Analysis)

| | | Placebo | h-SCP (SEQ ID No: 1) | | | | | | | |
|---|---|---|---|---|---|---|---|---|---|---|
| | | Infusion rate (ng/kg/min) | | | | | | | | |
| | | 0 | 0.3 | 1 | 3 | 9 | 18 | 36 | 45 | 54 |
| Heart | N | 7 | 2 | 4 | 8 | 7 | 9 | 5 | 2 | 2 |
| failure | Baseline, | 68.9 | 72.8 | 74.8 | 71.9 | 71.8 | 69.3 | 69.4 | 67.0 | 70.2 |
| subjects | mm Hg | (2.7) | (0.5) | (3.8) | (4.4) | (3.4) | (3.8) | (3.2) | (3.8) | (19.2) |
| | CFB, | 0.0 | −8.6 | −6.1 | −8.6 | −8.6 | −7.6 | −12.5 | −12.6 | −14.4 |
| | mm Hg | (1.5) | (3.4) | (1.8) | (2.0) | (3.1) | (2.1) | (1.6) | (2.4) | (5.3) |
| | Percent | 0.0 | −12.6 | −9.1 | −13.0 | −11.9 | −11.4 | −18.5 | −19.0 | −23.9 |
| | CFB | (2.2) | (4.6) | (2.5) | (3.0) | (3.9) | (3.2) | (2.3) | (4.0) | (10.6) |

Mean systemic vascular resistance and mean systemic vascular resistance index were increased from baseline in placebo, were variable at doses from 0.3 to 9 ng/kg/min, and were decreased from baseline at doses 18 ng/kg/min.

An echocardiography substudy was conducted to examine the impact of h-SCP (SEQ ID NO:1) on cardiodynamic parameters. Five subjects elected to participate in the echocardiography substudy. One subject received placebo and 4 subjects received h-SCP (SEQ ID NO:1) at doses ranging from 9 to 45 ng/kg/min during their last 2.5-hour infusion period when the echocardiogram was obtained. The one subject who received placebo had a decrease in their ejection fraction from 43.0% to 40.9%. The two subjects who received the lower doses of 9 and 36 ng/kg/min each had increases in their ejection fractions from 20% to 24.5% and from 25.0% to 30.3%, respectively. Both subjects who received 45 ng/kg/min had decreases in their ejection fractions from 36.0% to 34.7% and from 28.0% to 26.1%, respectively. Because of the small number of subjects who participated in this substudy and the varied dose administered the results are not conclusionary, but mainly indicative of the effect.

Healthy Subjects, 24- and 72-Hour Continuous IV Infusion, 54 ng/kg/min

Healthy subjects who received placebo had heart rates that decreased compared with baseline during the infusion. Subjects who received placebo had a mean decrease in heart rate of 5 to 10 bpm during the infusion from baseline heart rate (value obtained immediately before the infusion). A review of the heart rate data in these subjects indicated that their heart rates were 5 to 10 bpm higher at baseline compared with the day before infusion. This suggests that similar to above studies the subjects may have experienced anxiety before the start of the infusion that contributed to this increase in baseline heart rate values.

In contrast with subjects receiving placebo, who had a decrease in heart rate during the infusion, subjects receiving h-SCP (SEQ ID NO:1) at 54 ng/kg/min had an increase in heart rate of 5 to 10 bpm during the infusion compared with baseline. This increase in heart rate occurred rapidly within 15 minutes. The heart rate tended to decrease over the next 4 to 12 hours, but remained elevated relative to baseline until the infusion was discontinued after 24 or 72 hours. No notable differences in response were seen between male and female subjects.

Based on these observations it appears that h-SCP (SEQ ID NO:1) at 54 ng/kg/min was associated with an increase in heart rate from baseline particularly when compared with placebo.

Healthy subjects who received placebo had cardiac indices and cardiac outputs that decreased compared with baseline during the infusion. These decreases from baseline were apparently due to the decrease in heart rate during the placebo infusions since the stroke volume did not change during the infusion.

For subjects receiving h-SCP (SEQ ID NO:1) at doses 54 ng/kg/min, the effect on cardiac index, cardiac output, and stroke volume were variable and inconsistent. It is possible that the decreased time for diastolic filling that resulted from the higher heart rate may have decreased the stroke volume, cardiac output, and cardiac index in some subjects, while the increase in heart rate may have increased cardiac output and cardiac index in others.

No trends were observed in mean systolic and diastolic blood pressure in the placebo and 24-hour groups. Mean systolic and diastolic blood pressure were generally decreased from baseline in the 72-hour male and 72-hour female groups.

Mean systemic vascular resistance and mean systemic vascular resistance index were mostly increased from baseline in the placebo and 24-hour groups and were mostly decreased from baseline in the 72-hour male and 72-hour female groups.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating heart failure in a subject in need thereof, comprising administering to the subject a therapeutically effective amount of stresscopin-like peptide in a dose that does not exceed a stresscopin relative concentration of 7.2 ng/mL in the subject for a continuous period of more than about 15 minutes.

2. The method of claim 1, wherein the dosage that does not exceed a stresscopin relative concentration of 5.5 ng/mL in the subject for a continuous period or more than about 10 minutes.

3. The method of claim 1, wherein the dosage that does not exceed a stresscopin relative concentration of 4.7 ng/mL in the subject for a continuous period or more than about 10 minutes.

4. The method of claim 1, wherein the plasma concentration of said subject is substantially maintained between a stresscopin-relative concentration of about 0.1 ng/mL to about 7.2 ng/mL during the treatment.

5. The method of claim 4, wherein the plasma concentration of said subject is substantially maintained between a stresscopin-relative concentration of about 0.1 ng/mL to about 5.5 ng/mL during the treatment.

6. The method of claim 4, wherein the plasma concentration of said subject is substantially maintained between a stresscopin-relative concentration of about 0.1 ng/mL to about 4.7 ng/mL during the treatment.

7. The method of claim 1, wherein said stresscopin-like peptide is administered over a period of at least about 30 minutes.

8. The method of claim 1, wherein said dose is administered via a parenteral route.

9. The method of claim 8, wherein said parenteral route is selected from the group consisting of intravenous administration, subcutaneous administration, and intramuscular administration.

10. A method for treating heart failure in a subject in need thereof, said method comprising intravenously administering a stresscopin-like peptide at a stresscopin-relative dosing rate of between about 0.2 ng/kg/min to about 52 ng/kg/min over a time period of at least about 30 minutes.

11. The method of claim 10, wherein said stresscopin-like peptide is intravenously administered at a stresscopin-relative dosing rate of between about 0.2 ng/kg/min to about 36 ng/kg/min over a time period of at least about 30 minutes.

12. The method of claim 10, wherein said stresscopin-like peptide is intravenously administered at a stresscopin-relative dosing rate of between about 0.4 ng/kg/min to about 18 ng/kg/min over a time period of at least about 30 minutes.

13. The method of claim 10, wherein said stresscopin-like peptide is subcutaneously administered at a stresscopin-relative bolus dose of between 0.002 μg/kg to about 0.2 μg/kg.

14. The method of claim 10, wherein said stresscopin-like peptide comprises the amino acid sequence of SEQ ID NO. 1 or 29, said amino acid sequence of SEQ ID NO. 1 or 29 optionally conjugated at position 28 with 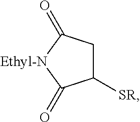
wherein R is the stresscopin-like peptide having the amino acid sequence of SEQ ID NO. 1 or 29, and S is the sulfur atom of the cysteine thiol group at position 28.

15. The method of claim 10, wherein said stresscopin-like peptide is intravenously administered at a dosing rate of between about 0.2 ng/kg/min to about 52 ng/kg/min over a time period of at least about 30 minutes.

16. The method of claim 14, wherein said stresscopin-like peptide is subcutaneously administered at a bolus dose of between 0.002 μg/kg to about 0.2 μg/kg.

17. The method of claim 1, wherein said dose comprises a peptide having the amino acid sequence of SEQ ID NO. 19, and S is the sulfur atom of the cysteine thiol group at position 18.

18. The method of claim 17, wherein said dose is intravenously administered at a dosing rate of between about 6 ng/kg/min to about 1700 ng/kg/min over a time period of at least about 30 minutes.

19. The method of claim 17, wherein said dose is subcutaneously administered at a bolus dose of between 0.01 μg/kg to about 1 μg/kg.

20. The method of claim 1, wherein said stresscopin-like peptide comprises polyethylene glycol (PEG) to a linker, wherein said linker is attached to the stresscopin-like peptide and the PEG weighs no more than about 80 kDa.

21. The method of claim 20, wherein said stresscopin-like peptide comprises a conjugate selected from 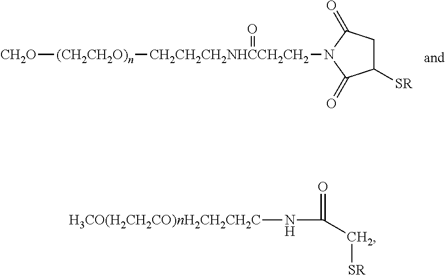
wherein n is an integer of about 460, R is a peptide having the amino acid sequence of SEQ ID NO. 29, and S is the sulfur atom of the cysteine thiol group at position 28.

22. The method of claim 21, wherein said dose is intravenously administered at a dosing rate of between about 20 ng/kg/min to about 5200 ng/kg/min over a time period of at least about 30 minutes.

23. The method of claim 21, wherein said dose is subcutaneously administered at a bolus dose of between 0.9 μg/kg to about 100 μg/kg.

24. The method of claim 1, wherein said stresscopin-like peptide is at least about 90% homologous to the peptide of SEQ ID NO:1.

25. The method of claim 1, wherein said stresscopin-like peptide is at least about 90% identical to the peptide of SEQ ID NO:1.
